(19) United States
(12) Patent Application Publication
(10) Pub. No.: US 2025/0327125 A1
(43) Pub. Date: Oct. 23, 2025

(54) SYSTEMS AND METHODS FOR ANALYSIS OF SAMPLES ASSOCIATED WITH NONALCOHOLIC FATTY LIVER DISEASE

(71) Applicant: THE REGENTS OF THE UNIVERSITY OF MICHIGAN, Ann Arbor, MI (US)

(72) Inventor: Elizabeth Speliotes, Ann Arbor, MI (US)

(21) Appl. No.: 18/870,228

(22) PCT Filed: Jun. 1, 2023
(86) PCT No.: PCT/US2023/067770

§ 371 Date: Nov. 27, 2024

Related U.S. Application Data
(60) Provisional application No. 63/347,799, filed on Jun. 1, 2022.
(60) Provisional application No. 63/377,471, filed on Sep. 28, 2022.

(51) Int. Cl. C12Q 1/6883

(52) U.S. Cl. CPC C12Q 1/6883; C12Q 2600/156; C12Q 2600/158

(57) ABSTRACT

Provided herein are systems and methods for analysis of biological samples to identify biomarkers associated with non-alcoholic fatty liver disease. For example, provided herein are molecular signatures that find use in characterizing samples to facilitate research, drug discovery, and treatment associated with nonalcoholic fatty liver disease.

### CROSS-REFERENCE TO RELATED APPLICATIONS

This application claims the benefit of U.S. Provisional Application Nos. 63/347,799, filed Jun. 1, 2022, and 63/377,471, filed Sep. 28, 2022, the contents of which are herein incorporated by reference in their entirety.

### STATEMENT REGARDING FEDERALLY SPONSORED RESEARCH OR DEVELOPMENT

This invention was made with government support under DK107904 awarded by the National Institutes of Health. The government has certain rights in the invention.

### SEQUENCE LISTING STATEMENT

The contents of the electronic sequence listing titled UM-39791-601.xml (Size: 27,011,446 bytes; and Date of Creation: May 31, 2023) is herein incorporated by reference in its entirety.

### FIELD

Provided herein are systems and methods for analysis of biological samples to identify biomarkers associated with nonalcoholic fatty liver disease. For example, provided herein are molecular signatures that find use in characterizing samples to facilitate research, drug discovery, and treatment associated with nonalcoholic fatty liver disease.

### BACKGROUND

Nonalcoholic fatty liver disease (NAFLD) is the most common liver disease worldwide and has no effective treatments. NAFLD is heritable.

With rising obesity rates, the prevalence of nonalcoholic fatty liver disease (NAFLD) has increased to epidemic proportions. NAFLD is caused by the deposition of excess fat in the liver (not due to alcohol), and can lead to advanced liver diseases including inflammation, fibrosis/cirrhosis (scarring), and hepatocellular carcinoma (HCC; liver cancer). NAFLD is also associated with metabolic diseases including dyslipidemia, hypertension, cardiovascular disease, and diabetes, though causal relationships have yet to be established. More than 90% of severely obese individuals suffer from advanced NAFLD, which is associated with a shorter lifespan. The disease imposes an annual direct medical cost of about $103 billion in the United States and will soon become the leading indication for liver transplantation in this country. The causes of NAFLD are poorly understood, and there are presently no effective treatments, making NAFLD treatment a large unmet medical need.

NAFLD is heritable and has identified variants associated with disease. However, these variants explain only about 20% of the heritability. What is needed are systems and methods to better analyze the disease to facilitate drug discovery and disease prevention and treatment.

### SUMMARY

Provided herein are systems and methods for analysis of biological samples to identify biomarkers associated with nonalcoholic fatty liver disease (NAFLD). For example, provided herein are molecular signatures that find use in characterizing samples to facilitate research, drug discovery, and treatment associated with nonalcoholic fatty liver disease.

In experiments conducted during the development of the invention, the largest genome-wide association meta-analysis of imaging and diagnostic code measured NAFLD to date was carried out. We identified a number of genome-wide significant NAFLD associated variants, a significant NAFLD associated gene, and confirmed ten additional, previously published liver function test (LFT) and NAFLD associated variants. These variants, and the genes and pathways they highlight, provide new insights into the pathogenesis of NAFLD, identify subtypes of disease, and create new genetic marker panels that can identify individuals at higher genetic risk of advanced liver disease and that facilitate research, drug discovery, and treatment of patients suffering from NAFLD.

For example, new NAFLD associated variants at TOR1B (Torsin Family 1 Member B), FTO (FTO Alpha-Ketoglutarate Dependent Dioxygenase), COBLL1 (Cordon-Bleu WH2 Repeat Protein Like 1)/GRB14 (Growth Factor Receptor Bound Protein 14), INSR (Insulin Receptor), SREBF1 (Sterol regulatory element-binding transcription factor 1), and PNPLA2 (Patatin Like Phospholipase Domain Containing 2), as well as reproducible NAFLD associated variants at APOE (Apolipoprotein E), MARC1 (Mitochondrial Amidoxime Reducing Component 1), GCKR (Glucokinase Regulator), TM6SF2 (Transmembrane 6 Superfamily Member 2), PNPLA3 (Patatin Like Phospholipase Domain Containing 3), GPAM (Glycerol-3-Phosphate Acyltransferase, Mitochondrial), TRIB1 (Tribbles Pseudokinase 1), MTTP (Microsomal Triglyceride Transfer Protein), ADH1B (Alcohol Dehydrogenase 1B (Class I), Beta Polypeptide), PTPRD (Protein Tyrosine Phosphatase Receptor Type D), andTMC4 (Transmembrane Channel Like 4)/MBOAT7 (Membrane Bound O-Acyltransferase Domain Containing 7), were identified.

Genes implicated by these variants play a role in mitochondrial, very-low-density lipoprotein (VLDL), cholesterol, and de novo lipogenesis processes. PheWAS analyses reveal at least seven subtypes of NAFLD. Genetic predisposition to NAFLD causally predisposes to cirrhosis and genetic predisposition to higher body mass index and waist circumference causally predisposes to NAFLD. Individuals at the top 10% and 1% of genetic risk have 3- to 6-fold increased risk of NAFLD, cirrhosis, and hepatocellular carcinoma. These genetic variants identify subtypes of disease, improve estimates of disease risk, and guide development of targeted therapeutics as well as identifying subject for appropriate interventions and preventative strategies.

For example, in some embodiments compositions, kits, systems, and methods are provided for analyzing the one or more variants. Variants are detected directly or indirectly. In some embodiments, direct methods comprise use of a molecular assay such as a hybridization assay (e.g., using one or more allele-specific primers or probes), a sequencing assay, a microarray, a cleavage assay, or the like. In some embodiments, indirect methods comprising detection of variants in linkage equilibrium with a variant, detections of altered gene expression relative to wild-type, or the like.

In some embodiments, the methods comprise analyzing a biological sample from a subject for one or more variants. In some embodiments, one or more (e.g., 1, 2, 3, 4, 5, 6, 7, 8, 9, 10, 11, 12, 13, 14, 15, 16, all) of the variants rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358 and mutations in MTTP are detected. In some embodiments, one or more of these variants is detected in combination with one or more other variants. In some such embodiments, the total number of variants detected or analyzed is less than 500, less than 200, less than 100, less than 50, or less than 25. In some embodiments, at least 10 of the listed variants are analyzed. In some embodiments, at least fifteen of the variants listed are analyzed. In some embodiments, at least 20 of the variants listed are analyzed. In some embodiments, only variants from the listed variants are analyzed. In other embodiments, additional variants not listed are analyzed in combination with one or more of the listed variants.

Any suitable sample may be used that contains nucleic acid amenable to analysis. In some embodiments, the biological sample is selected from the group consisting of blood, serum, plasma, saliva, tissue, hair, semen, and urine. In some embodiments, a biological sample is obtained from a subject suspected of having nonalcoholic fatty liver disease. Such suspicion may arise from any of any number of factors including, but not limited to, family history, obesity, signs or symptoms of disease, and a positive imaging or diagnostic test suggesting disease.

Also provided herein are methods of managing nonalcoholic fatty liver disease, comprising: analyzing a biological sample from a subject for one or more of the variants from the list of rs738408, rs58542926, rs429358, rs 1260326, rs28601761, rs4918722, rs2807834, rs7661964, rs1229984, rs7029757, rs17817449, rs79953491, rs112630404, rs626283, rs4561528, rs10756038, rs140201358, or a variant or marker in linkage disequilibrium therewith, and mutations in MTTP; generating a fatty liver disease risk score based on the presence or absence of said variants; and treating the subject with a nonalcoholic fatty liver disease intervention if said risk score indicates a predisposition to or presence of nonalcoholic fatty liver disease. In some embodiments, the risk score is calculated using an algorithm that accounts for each of the analyzed variants. In some embodiments, the risk score further is based on one or more of blood count, liver enzyme test data, liver function test data, hepatitis A test data, hepatitis C test data, celiac disease screening test data, fasting blood sugar, hemoglobin AIC data, and lipid profile data. In some embodiments, the risk score further is based on one or more of; age, gender, and/or body composition. In some embodiments, the risk score further is based on one or more of abdominal ultrasound data, computerized tomography (CT) scanning data, magnetic resonance imaging (MRI) data, transient elastography data, and magnetic resonance elastography data. In some embodiments, the treating comprises applying a weight loss regime. In some embodiments, the treating comprises liver transplantation. In some embodiments, the treating comprises administration of a pharmaceutical agent. In some embodiments, the pharmaceutical agent is one or more of: an essential phospholipid (e.g., polyenylphosphatidylcholine); an anti-diabetic agent (e.g., insulin, metformin, pioglitazone, glucagon-like peptide-1 (GLP-1) agonists, sodium-glucose cotransporter-2 (SGLT-2) inhibitors, thiazolidinediones (TZD), obeticholic acid, ursodeoxycholic acid, RG-125); a dietary supplement (e.g., vitamin E, silymarin, S-adenosyl-L-methionine (SAMe), glutathione, glycyrrhizic acid); an antifibrotic agent (e.g., RAS blockers such as angiotensin-converting enzyme inhibitors (ACEIs) and angiotensin II receptor blockers (ARBs), pentoxifylline, larsucosterol, galectin-3 inhibitors, cenicriviroc); and an anti-obesity agent (e.g., sibutramine).

Further provided herein are systems (e.g., kits, reactions mixtures, etc.) comprising: a set or reagents that specifically detect one or more variants from the list of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, or a variant or marker in linkage disequilibrium therewith, and mutations in MTTP. In some embodiments, the system detects a total of less than 500, less than 200, less than 100, less than 50, or less than 25 variants. In some embodiments, the reagents comprise one or more primers or probe specific for the variants (e.g., primers or probes useful in allele-specific PCR or similar assays). In some embodiments, the reagents comprising nucleic acid sequence reagents. In some embodiments, the reagents comprise a microarray (e.g., a hybridization based microarray).

Also provided herein is a non-transitory computer-readable storage medium comprising an instruction, wherein when the instruction is run by at least one computer processor, wherein the at least one processor performs operations comprising one or more or each of the steps: a) receiving data identifying the presence or absence of a variant in a biological sample from at least one of rs738408, rs58542926, rs429358, rs 1260326, rs28601761, rs4918722, rs2807834, rs7661964, rs1229984, rs7029757, rs17817449, rs79953491, rs112630404, rs626283, rs4561528, rs10756038, rs140201358, or a variant or marker in linkage disequilibrium therewith, and mutations in MTTP; b) generating a nonalcoholic fatty acid liver disease risk score from the data; and c) displaying or reporting said risk score. The displaying may comprise generating a written or electronic report for use by a physician, a researcher, a patients, or any other desired format.

Further provided herein are methods of diagnosing fatty liver disease or predisposition to fatty liver disease comprising: analyzing a biological sample from a subject for one or more variant from the list of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, or a variant or marker in linkage disequilibrium therewith, and mutations in MTTP

### BRIEF DESCRIPTION OF FIGURES

FIG. 1 shows the characteristics of a subset of GOLDPlus genome-wide significant variants in GOLD ancestry-based cohorts. For each variant the characteristics are shown for the GOLD ancestry-based analysis including: associated gene, NAFLD increasing effect allele (EA), effect allele frequency (EAF), effect/beta and 95% confidence interval, Cochran's Q heterogeneity 12 metric and heterogeneity p-value, EA p-value (P), and sample size (N). Results are for meta-analysis of GOLD European ancestry (red), African ancestry (blue), Hispanic ancestry (green), Chinese ancestry (purple), and all ancestries pooled (black).

FIG. 2 shows the effects of NAFLD associated variants on other human diseases and traits. Associations between NAFLD associated variants and diseases are shown as Z-scores in the heatmap. White horizontal bars between the groups in the heatmaps were used to separate each k-means cluster. Red indicates that the NAFLD-increasing allele has increased association with the disease/trait, blue indicates decreased association, and white indicates no significant association. A horizontal bar atop the heatmap corresponds to overall groupings of the disease/traits in the key. Gray boxes on the vertical axis indicate the overall protein localization of the genes in each cluster.

FIGS. 3A-3C show the associations between NAFLD polygenic risk score with NAFLD, cirrhosis, and HCC in an independent cohort. Association between percentile of GOLDPlus NAFLD polygenic risk score on the independent MGI cohort on NAFLD (FIG. 3A), cirrhosis (FIG. 3B), or HCC (FIG. 3C). All results are depicted as odds ratios for NAFLD, cirrhosis, or HCC relative to individuals in the 0-10th percentile of polygenic risk score, adjusted for sex, age, age², and PCs 1-10. Error bars represent 95% confidence intervals.

FIG. 4 shows GOLDPlus NAFLD measures meta-analysis study design.

FIGS. 5A-5Q are LocusZoom plots of index GOLDPlus Significant Variants. Index variant is labeled in purple and when applicable exonic variant in LD with index variant is labeled in red and 1000G EUR ancestry linkage disequilibrium structure utilized is used. (FIG. 5A) rs738408-PNPLA3, (FIG. 5B) rs58542926-TM6SF2, (FIG. 5C) rs429358-APOE, (FIG. 5D) rs1260326-GCKR, (FIG. 5E) rs28601761-TRIB1, (FIG. 5F) rs4918722-GPAM, (FIG. 5G) rs2807834-MARC1, (FIG. 5H) rs7661964-MTTP, (FIG. 5I) rs7029757-TOR1B, (FIG. 5J) rs1229984-ADH1B, (FIG. 5K) rs17817449-FTO, (FIG. 5L) rs79953491-COBLL1, (FIG. 5M) rs112630404-INSR, (FIG. 5N) rs626283-TMC4/MBOAT7, (FIG. 50) rs4561528-SREBF1, (FIG. 5P) rs10756038-PTPRD, and (FIG. 5Q) rs140201358-PNPLA2.

FIG. 6 shows European GOLDPlus NAFLD measures meta-analysis schematic.

FIG. 7 shows characteristics of GOLDPlus genome-wide significant variants in GOLD ancestry-based cohorts. For each variant the characteristics are shown for the GOLD ancestry-based analysis including: associated gene, NAFLD increasing effect allele (EA), effect allele frequency (EAF), effect/beta and 95% confidence interval, Cochran's Q heterogeneity 12 metric and heterogeneity p-value, EA p-value (P), and sample size (N). Results are for meta-analysis of GOLD European ancestry (red), African ancestry (blue), Hispanic ancestry (green), Chinese ancestry (purple), and all ancestries pooled (black).

FIG. 8 shows characteristics of GOLDPlus genome-wide significant variants in GOLD sex-specific cohorts. For each variant the characteristics are shown for the GOLD sex-specific analysis including: associated gene, NAFLD increasing effect allele (EA), effect allele frequency (EAF), effect/beta and 95% confidence interval, Cochran's Q heterogeneity 12 metric and heterogeneity p-value, EA p-value (P), and sample size (N). Results are for meta-analysis of GOLD cohort males (blue), females (red), and pooled sexes (black).

FIG. 9 shows DEPICT analysis of biological enrichment of NAFLD associated variants. Physiological system, cell, and tissue enrichment of NAFLD associated genetic variants. Height of the bar represents-log₁₀p-value. Orange shading represents statistical significance at false discovery rate (FDR)<0.05.

FIG. 10 show K-Means clustering of PheWAS results for NAFLD associated variants. Grid shows variant cluster assignment for K-means clusters of k=4, k=5, k=6, and k=7. Variants assigned to each cluster are shown in the color-coded legends.

FIGS. 11A-11D shows two-sample Mendelian randomization analysis for casual associations between NAFLD and fibrosis/cirrhosis and esophageal varices. Effect size is shown by a red point and 95% confidence interval by a red line for MR EGGER and inverse variance weighted methods for (FIG. 11A) NAFLD exposure (GOLD cohort, N=10 instruments) and K74: fibrosis/cirrhosis outcome (UKBB) and (FIG. 11B) NAFLD exposure (GOLD cohort, N=10 instruments) and 185: esophageal varices outcome (UKBB). The crosshairs on the plots in FIGS. 11C and 11D represent the 95% confidence intervals for each SNP-NAFLD or SNP-outcome association for (FIG. 11C) NAFLD exposure (GOLD cohort, N=10 instruments) and K74: fibrosis/cirrhosis outcome (UKBB) and (FIG. 11D) NAFLD exposure (GOLD cohort, N=10 instruments) and 185: esophageal varices outcome (UKBB).

FIGS. 12A-12D show two-sample Mendelian randomization analysis for casual associations between BMI, waist circumference, and NAFLD. Effect size is shown by a red point and 95% confidence interval by a red line for MR EGGER and inverse variance weighted methods for (FIG. 12A) waist circumference GWAS (UKBB, N=302 instruments (independent SNPs p-value <5E-08)) and GOLD cohort outcome (FIG. 12B) BMI GWAS (UKBB, N=315 instruments (SNPs p-value <5E-08)) and GOLD cohort outcome. The crosshairs on the plots in FIGS. 12C and 12D represent the 95% confidence intervals for each SNP-NAFLD or SNP-outcome association for (FIG. 12C) waist circumference GWAS (UKBB, N=211 instruments) and GOLD cohort outcome and (FIG. 12D) BMI GWAS (UKBB, N=283 instruments) and GOLD cohort outcome.

FIGS. 13A and 13B show convolutional neural network schematic for UKBB MRI liver imaging (PCC values). Scatter plot of predicted UKBB MRI-PDFF values versus “true” UKBB MRI-PDFF values (as determined by Perspectum Diagnostics). Pearson correlation coefficients are shown for (FIG. 13A) gradient echo image protocol and (FIG. 13B) IDEAL image protocol.

FIG. 14 is a chart showing the effects of NAFLD associated variants in individual GOLDPlus meta-analysis datasets.

FIG. 15 is a table outlining the association of the identified biomarkers for 7 metabolic groups.

FIG. 16 is a schematic showing treatments for various indications of NAFLD.

FIGS. 17A-17F show the genetic and environmental factors associated with progression to cirrhosis in Michigan Genomics Initiative. Models were run as Fine-Gray competing risk analyses. Diabetes status (FIG. 17A), obesity status (FIG. 17B), and alanine aminotransferase (ALT) (FIG. 17C), with upper limited of normal (ULN) defined as 19 U/L in women and 30 U/L in men. PNPLA3-rs738409 genotype (FIG. 17D), TRIB1-rs28601761 genotype (FIG. 17E) and cirrhosis polygenic risk score (FIG. 17F), divided into quartiles (Q), with Q1 indicating the lowest quartile.

FIGS. 18A and 18B show PNPLA3 genotype and diabetes status identify a subgroup of patients with low FIB4 with cirrhosis incidence comparable to that of patients with high FIB4 in the Michigan Genomics Initiative (FIG. 18A) and the UK Biobank (FIG. 18B). Models were run as a Fine-Gray competing risk analysis. Patients were divided into three groups: high FIB4, low FIB4 with diabetes [(+) DM] and PNPLA3-rs738409-GG genotype [(+) PNPLA3], and low FIB4 with diabetes and PNPLA3-rs738409-CC or-CG genotype [(−) PNPLA3]. High FIB4 was defined as >=2.67 while low was defined as <2.67. Hazard ratios (HRs) and p values are shown at the top left of each graph and represent effects of each group after adjustment for age, sex, and principal components 1-10.

### DETAILED DESCRIPTION

Disclosed herein are a number of loci that include several genes not previously known to be associated with nonalcoholic fatty liver disease (NAFLD). The effect of these variants on NAFLD was congruent across study, ancestry, sex, and alcohol intake. However, some of the associated variants have EAF differences across ancestries which are consistent with differences in population burden of NAFLD. An additional gene, MTTP, was associated with NAFLD via gene-based analysis. Tissue and pathways enrichment analyses of these associations identified liver, lipid, cholesterol, steroid, alcohol, and monocarboxylic acid processes as being enriched. PheWAS analysis resulted in at least seven subtypes/clusters of NAFLD associated variants and implicated genes from these analyses that play a role in mitochondrial, VLDL, cholesterol, and de novo lipogenesis processes. A risk score of the NAFLD-associated genetic variants improved risk predictions when added to age, sex, and clinical factors in identifying people with elevated risk of NAFLD, cirrhosis, and hepatocellular carcinoma (HCC).

Carrying out the analysis across imaging, ICD-based, and NLP-based diagnosis of NAFLD provided substantial advantages over traditional histology-or single modality-based GWAS. These measures are less expensive, less invasive, and more ethically applicable to asymptomatic individuals in the general population than liver biopsy. The inclusion of non-histology-measured NAFLD increased power and decreased ascertainment bias. Furthermore, by assessing heterogeneous effects of variants across multiple modalities, a variant associated with other types of liver disease, such as glycogen storage disease, that can be misdiagnosed as NAFLD can be identified and removed from the analysis. Also disclosed are machine learning methods to predict MRI-PDFF from abdominal MRI images which can be used to facilitate future studies incorporating imaging analysis for NAFLD and other imaging endpoints.

In addition to identifying novel variants associated with NAFLD, the combined effect of the single variants using MR, pathway analysis, and PRS. MR analysis suggested that obesity, as measured by high BMI or waist circumference, is causally related to development of NAFLD, but not the reverse. However, MR showed hepatic steatosis is causally related to fibrosis/cirrhosis.

Taken together, the genetic variants can identify individuals at higher risk of having NAFLD, cirrhosis and HCC. In an independent cohort, the risk score identified individuals at high risk of NAFLD, cirrhosis, and HCC in the top 5% of the risk score. The risk score added predictive ability when combined with other clinical risk factors, showing that it finds use to identify high-risk individuals who might benefit from more intense management of NAFLD risk factors.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%, 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5xSSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46:453 (1960) and Doty et al., Proc. Natl. Acad. Sci. USA, 46:461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

“Hybridization probes” are nucleic acids capable of binding in a base-specific manner to a complementary strand of nucleic acid. Such probes include nucleic acids and peptide nucleic acids. Hybridization is usually performed under stringent conditions which are

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer, but typically ranges from 15 to 30 nucleotides. A primer sequence need not be exactly complementary to a template, but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

The nucleic acids, including any primers, probes and/or oligonucleotides can be synthesized using a variety of techniques currently available, such as by chemical or biochemical synthesis, and by in vitro or in vivo expression from recombinant nucleic acid molecules, e.g., bacterial or retroviral vectors. For example, DNA can be synthesized using conventional nucleotide phosphoramidite chemistry or other methodologies well known in the art. In addition, the nucleic acids can comprise uncommon and/or modified nucleotide residues or non-nucleotide residues, such as those known in the art.

The terms “polymorphism” or “variant” refers to the occurrence of two or more genetically determined alternative sequences or alleles in a population. Each divergent sequence is termed an allele, and can be part of a gene or located within an intergenic or non-genic sequence. A diallelic polymorphism has two alleles, and a triallelic polymorphism has three alleles. Diploid organisms can contain two alleles and may be homozygous or heterozygous for allelic forms. The first identified allelic form is arbitrarily designated the reference form or allele; other allelic forms are designated as alternative or variant alleles. The most frequently occurring allelic form in a selected population is typically referred to as the wild-type form.

As used herein, “treat,” “treating,” and the like means a slowing, stopping, or reversing of progression of a disease or disorder. The term also means a reversing of the progression of such a disease or disorder. As such, “treating” means an application or administration of methods to a subject, where the subject has a disease or a symptom of a disease, where the purpose is to cure, heal, alleviate, relieve, alter, remedy, ameliorate, improve, or affect the disease or symptoms of the disease.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Analyzing Polymorphisms

Provided herein are methods comprising analyzing a biological sample from a subject for one or more of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, and mutations in MTTP.

The analysis described herein identified several genome-wide significant variants associated with hepatic steatosis and NAFLD, including rs738408-PNPLA3, rs58542926-TM6SF2, rs429358-APOE, rs1260326-GCKR, rs28601761-TRIB1, rs4918722-GPAM, rs2807834-MARC1, rs7661964-MTTP, rs7029757-TOR1B, rs1229984-ADH1B, rs17817449-FTO, rs79953491-COBLL1, rs112630404-INSR, rs626283-TMC4/MBOAT7, rs4561528-SREBF1, rs10756038-PTPRD, and rs140201358-PNPLA2.

The analyzed polymorphisms may be selected to include at least one polymorphism from each of the seven distinct clusters. In some embodiments, polymorphisms may contain at least one polymorphism from each of the significant variants and extended variants as shown in Table 1. In some embodiments, the polymorphisms may comprise at least two or all of the significant variants as shown in Table 1.

Presently the PRS is a composite of multiple SNPs weighted by the Beta of effect in the GOLD consortium as below with allele 1 being the effect allele and the beta being the weight. This is multiplied by the number of alleles (per individual) and summed to get the PRS per individual.

The gene-based analyses identified multiple variants in MTTP that promote NAFLD. MTTP is a well-known gene that transfers phospholipids and triacylglycerols to nascent apoB for the assembly of lipoproteins. The absence of MTTP is known to cause the Mendelian disease abetalipoproteinemia which causes malabsorption of in the digestive track resulting in fatty liver and other health issues. The mutations in MTTP may include, but are not limited to, G661S, Q244E, E98D, and N166S.

The present invention provides a method for diagnosing fatty liver disease or predisposition to fatty liver disease or related diseases or conditions. The presence of such a polymorphisms or mutations can be regarded as indicative of an individual's risk (increased or decreased) for the disease, especially in individuals who lack other predisposing or protective polymorphisms for the same disease. Even in cases where the predictive contribution of a given polymorphism is relatively minor by itself, overall assessment of the polymorphisms allows diagnosis with a much higher degree of certainty and reliability.

The present invention further provides a method of managing nonalcoholic fatty liver disease. Nonalcoholic fatty liver disease (NAFLD) is an umbrella term for a range of liver conditions affecting people who drink little to no alcohol. Some individuals with NAFLD can develop nonalcoholic steatohepatitis (NASH), an aggressive form of fatty liver disease, which is marked by liver inflammation and may progress to advanced scarring (cirrhosis), liver failure, or some forms of liver cancer. This damage is similar to the damage caused by heavy alcohol use. The methods disclosed herein may comprise managing the progression of nonalcoholic fatty liver disease to prevent a more aggressive form of liver disease. By extension, the methods disclosed herein may further act as an indication or prognosis of the risk of liver inflammation, liver scarring (cirrhosis), liver failure, or some forms of liver cancer.

The risk score may be calculated using an algorithm that accounts for one or more or each of the analyzed polymorphisms. The risk score may be calculated using non-weighted or weighted sums of risk polymorphisms using effect sizes from genome-wide association studies as their weights or effects of the particular polymorphism on the score. For example, those polymorphisms with inherently higher risk are weighted differently than those polymorphisms with lower individual risk.

The risk score may be based on other factors outside of the genetic polymorphisms described herein. Other factors may include the general health of the subject, previously identified disease in close family members, or other related identified disease or disorders. For example, risk factors may include high cholesterol, high levels of triglycerides in the blood, obesity, polycystic ovary syndrome, sleep apnea, diabetes, hypothyroidism, hypopituitarism, age, and concentration or abundance of abdominal body fat.

In some embodiments, risk score further is based on one or more of blood count, liver enzyme test data, liver function test data, hepatitis A test data, hepatitis C test data, celiac disease screening test data, fasting blood sugar, hemoglobin A1C data, and lipid profile data. In some embodiments, the risk score further is based on one or more of abdominal ultrasound data, computerized tomography (CT) scanning data, magnetic resonance imaging (MRI) data, transient elastography data, and magnetic resonance elastography data.

The risk score may be a measure of an individual risk of nonalcoholic fatty liver disease or related diseases in comparison to an average individual of a population or subset of population. For example, the score may be in comparison to any other individual or an individual with a similar ethnic background, age, sex, or prior health condition.

The risk score may be used to align a subject's level of disease with appropriate treatments. For examples, subjects with a specific disease phenotype may be linked to specific treatments for that subtype which results in the best management of the disease or lacks unwanted side effects or long-term complications.

The risk score may be output or displayed in any number of formats, including reports with bins, a color or grayscale gradient, a thermometer, a gauge, a histogram, or a bar graph. The risk score may provide a numerical output which is associated with low, medium, or high risk of NAFLD. Alternatively, or in addition, the risk score may be output as a rank score in a populations, such as a percentile of risk within a certain population. The risk score may be output with any proposed treatment recommendations or follow-up procedures to further assess risk. The risk score may be used to classify an individual into disease subtypes based on the at least seven subtypes/clusters of NAFLD associated variants and implicated genes from the analysis disclosed herein.

The risk score may further indicate the need or the type of treatment for an individual suspected to have or at risk of developing nonalcoholic fatty liver disease. Treatments for nonalcoholic fatty liver disease include those known in the art to reduce risk and include lifestyle changes, surgery, or medicament regimes. In some embodiments, the treatments include adoption of a healthy diet and exercise program, optionally as part of a weight loss regime, control of blood sugar, cholesterol lowering medications, and abstaining from alcoholic drinks. In some embodiments, treating includes liver transplantation. In some embodiment, treating comprises administration of one or more active agents. In some embodiments, the active agent is selected from: an essential phospholipid (e.g., polyenylphosphatidylcholine); an anti-diabetic agent (e.g., insulin, metformin, pioglitazone, glucagon-like peptide-1 (GLP-1) agonists, sodium-glucose cotransporter-2 (SGLT-2) inhibitors, thiazolidinediones (TZD), obeticholic acid, ursodeoxycholic acid, RG-125); a dietary supplement (e.g., vitamin E, silymarin, S-adenosyl-L-methionine (SAMe), glutathione, glycyrrhizic acid); an antifibrotic agent (e.g., RAS blockers such as angiotensin-converting enzyme inhibitors (ACEIs) and angiotensin II receptor blockers (ARBs), pentoxifylline, larsucosterol, galectin-3 inhibitors, cenicriviroc); an anti-obesity agent (e.g., sibutramine); or any combination thereof.

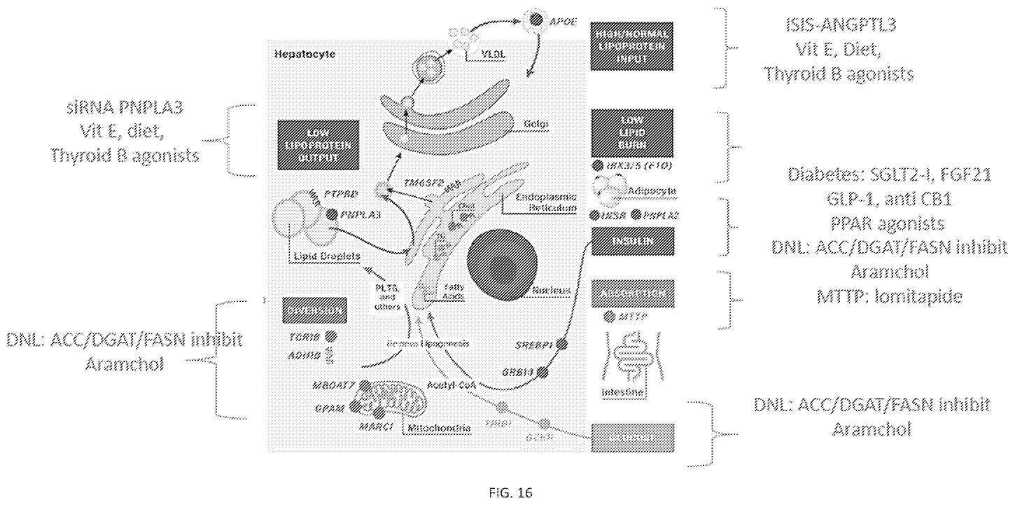

In some embodiments, the treating includes PNPLA3 siRNA, vitamin E administration, diet control, and Thyroid B agonists, for example when the patient is suspected to have or is at risk of low lipoprotein output. In some embodiments, the treating inhibitors of an acetyl-CoA carboxylase (ACC), Acyl-coenzyme A: diacylglycerol acyltransferase (DGAT), fatty acid synthase (FASN), or inhibitors of SCD1 (e.g., synthetic fatty-acid/bile-acid conjugate (FABAC), e.g., Aramchol) for example when the patient is suspected to have or is at risk of diversion of TG and phospholipids to lipid droplets or excess glucose conversion to fatty acids. In some embodiments, the treating includes ISIS-ANGPTL3, an antisense inhibitor to angiopoietin-like 3, vitamin E administration, diet control, and Thyroid B agonists, for example when the patient is suspected to have or is a risk of high or normal lipoprotein output. In some embodiments, the treating includes agonists of SGLT2-I (Sodium/glucose cotransporter-2), FGF21 (Fibroblast growth factor 21), glucagon-like peptide 1 (GLP1), anti-CB1/PPAR agonists (e.g., cannabinoid CB1 receptor antagonists and/or peroxisome proliferator-activated receptor agonists), inhibitors of microsomal triglyceride transfer protein (MTP or MTTP) (e.g., lomitapide), for example when the patient is suspected to have or is at risk of diabetes, insulin resistance, increases in fatty acids, or de novo lipogenesis (DNL). See, for example, FIG. 16.

In some embodiments, the treatments include modulating transcription, and thereby expression, of one or more target genes. For example, the treatments may include activation or repression of transcription of one or more target genes as listed in Table 7. In some embodiments, the treatments include knocking out one or more target genes. For example, the treatments may include knocking out one or more target genes as listed in Table 7.

In some embodiments, transcription of the target gene is modulated by administering a clustered regularly interspaced short palindromic repeats (CRISPR)/CRISPR associated (Cas) protein system for use in CRISPR interference (CRISPRi) or CRISPR activation (CRISPRa) (see, e.g., Konermann et al. Nature. 2014 Dec. 10. doi: 10.1038/nature14136; Qi, L. S., et al. (2013). Cell. 152 (5): 1173-83; Gilbert, L. A., et al., (2013). Cell. 154 (2): 442-51; and Maeder et al. Nat Methods 10 (10): 977-979 (2013)).

Cas proteins binding of specific DNA sequences through guide RNA can naturally result in a transcription block, a process termed CRISPR interference (CRISPRi). For use in mammalian cells, CRISPRi is even more effective when transcriptional repressor domains are tethered to the Cas protein. Transcriptional repressors may inhibit transcription via: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof. For example, transcriptional repressors such as the Kriippel associated box (KRAB or SKD); KOX1 repression domain; the Mad mSIN3 interaction domain (SID); the ERF repressor domain (ERD); histone lysine methyltransferases such as Pr-SET7/8, SUV4-20H1, RIZ1, and the like; histone lysine demethylases such as JM JD2 A/JHDM3 A, JMJD2B, JMJD2C/GASCI, JMJD2D, JARID 1 A/RBP2, JARIDIB/PLU-1, JARIDIC/SMCX, JARIDID/SMCY; histone lysine deacetylases such as HDAC1, HDAC2, HDAC3, HDAC8, HDAC4, HD AC 5, HDAC7, HDAC9, SIRT1, SIRT2, HDAC11; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), MET1, ZMET2, CMT1; periphery recruitment elements such as Lamin A and Lamin B; and functional domains thereof.

CRISPR/Cas systems can also be used to activate gene expression, in an approach termed CRISPR activation (CRISPRa). CRISPRa constructs generally utilize a Cas protein to recruit more than one transcription activation domain with a single gRNA. The activation domains may promote transcription via: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. For example, VP 16; VP64; VP48; VP 160; p65 subdomain (e.g., from NFkB); an activation domain of EDLL; TAL activation domain; histone lysine methyltransferases such as SETIA, SETIB, MLLI to 5, ASHI, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, PI 60, CLOCK; DNA demethylases such as Ten-Eleven Translocation (TET) dioxygenase 1 (TET1CD), TET1, DME, DML1, DML2, and ROS1; and functional domains thereof.

The Cas protein can recruit repressor or activation domains using direct fusions or protein linkers (e.g., SunTag). Alternatively, activation domains can be recruited using nucleic acid approaches, a guide RNA having binding motifs (e.g., MS2) recruits effector domains fused to RNA-motif binding proteins.

Any Cas protein that employs gRNA specific binding to bind to a specific target sequence can be utilized with the systems for CRISPRa and CRISPRi. Usually, a nuclease deficient version of a Cas protein is utilized, for example dCas9, a nuclease-dead Cas9 protein, but other Cas proteins can also be utilized in the methods herein, such as Cas3 and Cas12a.

In some embodiments, transcription of the target gene is knocked out by administering a CRISPR/nuclease protein system, e.g., CRISPR/Cas9, referred to as CRISPR-KO. An insertion or deletion induced by a single guide RNA (gRNA) is often used to generate knock-out cells. For example, a guide RNA targets Cas9 to a target gene, where it creates a double-stranded break (DSB). Cells can survive a DSB when an error-prone repair mechanism like nonhomologous end joining (NHEJ) results in insertion or deletion of one or more base pairs, precluding further binding of the gRNA. Such repairs can result in frameshift mutations and thereby disrupt gene function, oftentimes resulting in functional knockouts.

The CRISPR/Cas systems comprise a guide RNA specific to a target gene to be modulated. The target gene may be any of those listed in Table 7, and the CRISPR/Cas system may comprise any of those gRNAs for CRISPRa, CRISPRi, and CRISPR-KO as indicated in Table 7.

The CRISPR/Cas systems, including Cas proteins and gRNAs, or polynucleotides encoding thereof, may be delivered by any suitable means. Methods of delivering polypeptides and polynucleotides to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110 (6): 2082-2087, incorporated herein by reference); or viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, polynucleotides can be delivered by any method appropriate for introducing nucleic acids into a cell. In some embodiments, the polynucleotide is a DNA molecule. In some embodiments, the CRISPR/Cas system is provided in a DNA vector. In some embodiments, the CRISPR/Cas system is provided as an RNA molecule.

Additionally, delivery vehicles such as nanoparticle- and lipid-based polynucleotide or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1;459 (1-2): 70-83), incorporated herein by reference.

The risk score may also be used for selection (e.g., inclusion or exclusion) for a clinical trial. For example, subjects with a specific risk score may be included for a clinical trial to specifically study those individuals at an increased risk for nonalcoholic fatty acid liver disease, e.g., a genetic enrichment trial. Alternatively, subjects with a specific risk score may be excluded for a clinical trial to avoid potential interference with clinical trial analysis.

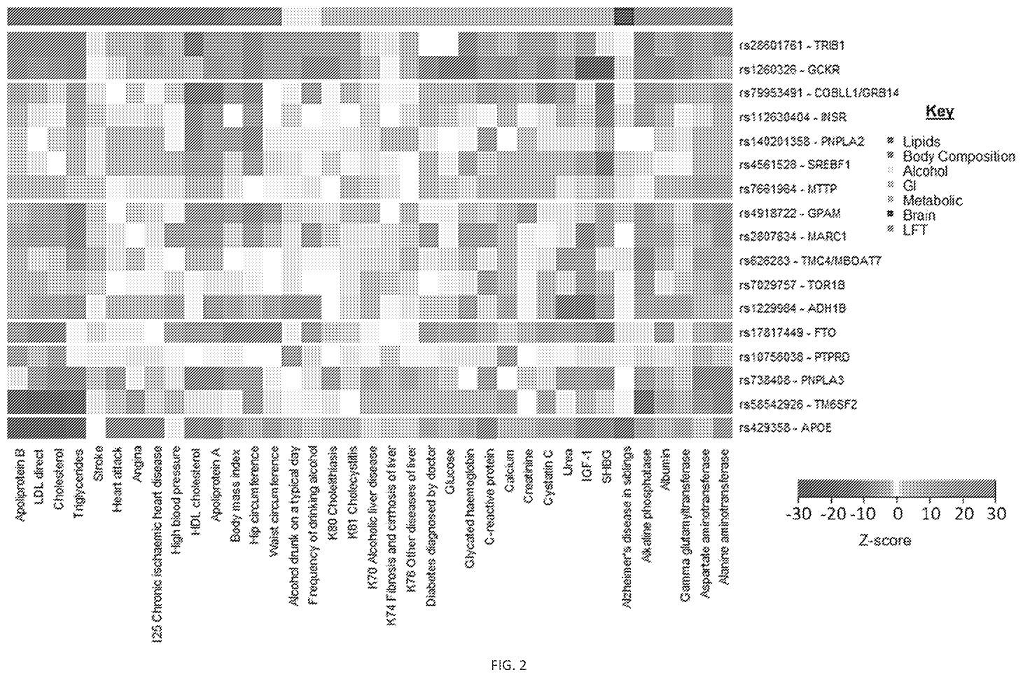

In some embodiments, the presence of such a polymorphisms or mutations can be regarded as indicative of an individual's risk (increased or decreased) for other diseases and conditions. As shown in FIG. 2, many of the polymorphisms or mutations had effects on metabolic and anthropometric traits such as lipid concentrations, cardiovascular disease, body mass index, waist/hip circumference, and liver enzyme levels.

In some embodiments, select polymorphisms or mutations are associated with higher low-density lipoprotein (LDL) and triglycerides (TG), increased risk of cardiovascular, lower high-density lipoprotein (HDL), and lower body mass index (BMI) and waist/hip circumference. In some embodiments, select polymorphisms or mutations are associated with higher LDL and TG and higher HDL. In some embodiments, select polymorphisms or mutations are associated with lower LDL, strongly increased risk of liver fibrosis/cirrhosis, and lower or no difference in alkaline phosphatase. In some embodiments, select polymorphisms or mutations are associated with decreased LDL and TG.

In some embodiments, rs28601761 and rs1260326 may be indicative of a decreased level of risk for cholelithiasis and/or cholecystitis. In some embodiments, rs1260326 may be associated with lower insulin-like growth factor 1 (IGF1) and sex hormone binding globulin (SHBG) levels. In some embodiments, rs429358 may be indicative of a decreased level of risk for familial Alzheimer's disease and LDL cholesterol.

The biological sample for analysis in the disclosed methods may be obtained from any suitable biological source, such as, a swab or brush, a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like, or a tissue or cell sample including, but not limited to, hair, skin, blood, biopsies of the kidney, or liver or other organs or tissues, or sources such as saliva, cheek scrapings, urine, amniotic fluid or CVS samples. In some embodiments, the biological sample is selected from the group consisting of blood, serum, plasma, saliva, tissue, hair, semen, and urine.

The sample can be obtained from a subject using routine techniques known to those skilled in the art, and the sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human). Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human. In some embodiments, the subject is suspected of having nonalcoholic fatty liver disease.

A polymorphism as described herein may be detected directly or indirectly. Direct detection methods may include inspecting a data set indicative of genetic characteristics derived from analysis of the individual's genome. A data set of genetic characteristics of the individual may include, for example, a listing of single nucleotide polymorphisms in the individual's genome or a complete or partial sequence of the individual's genomic DNA. Inspection of the data set including all or part of the individual's genome may optimally be performed by computer inspection. Screening may further comprise the step of producing a report identifying the individual and the identity of alleles at the site of at least one or more polymorphisms. Alternatively, the methods include obtaining and analyzing a nucleic acid sample (e.g., DNA or RNA) from an individual to determine whether the DNA contains informative polymorphisms, such as by combining a nucleic acid sample from the subject with one or more polynucleotide probes capable of hybridizing selectively to a nucleic acid carrying the polymorphism or sequencing the region of the DNA containing the polymorphisms. One skilled in the art will recognize that any one of the commonly available hybridization, amplification and array assay formats can readily be adapted to detect the polymorphisms disclosed herein.

In some embodiments, the polymorphisms are detected by a sequencing assay. The sequence assay may be conducted by any means known in the art, such as the dideoxy chain termination method. In some embodiments, the sequencing assay is performed using high-throughput sequence methods. Following sequencing, the data may be aligned or other analyzed for the presence of the polymorphisms. Methods of alignment of sequences for comparison purposes are well known in the art.

In some embodiments, the polymorphisms may be detected by an amplification-based assay in which a polymorphism-specific primer hybridizes to a region on a target nucleic acid molecule that overlaps the polymorphism and only primes amplification of that form to which the primer exhibits perfect complementarity. This primer is used in conjunction with a second primer that hybridizes at a distal site. Amplification proceeds from the two primers, producing a detectable product that indicates the polymorphism is present in the test sample. A control is usually performed with a second pair of primers, one of which shows one or more mismatches at the polymorphic site and the other of which exhibits perfect complementarity to a distal site. The mismatches prevent amplification or substantially reduce amplification efficiency, so that either no detectable product is formed or it is formed in lower amounts or at a slower pace. Amplification assays are well-known in the art including polymerase chain reaction, ligase chain reactions, strand displacement assays, and the like.

In a hybridization-based assay, probes can be designed that hybridize to a segment of target DNA from one individual but do not hybridize to the corresponding segment from another individual due to the presence of different polymorphic forms in the respective DNA segments. Hybridization conditions should be sufficiently stringent that there is a significant detectable difference in hybridization intensity, and preferably an essentially binary response, whereby a probe hybridizes to only one of the loci or significantly more strongly to one loci. A probe may be designed to hybridize to a target sequence that contains a polymorphism anywhere along the sequence of the probe. However, the probe is preferably designed to hybridize to a segment of the target sequence such that the polymorphism aligns with a central position of the probe (e.g., a position within the probe that is at least three nucleotides from either end of the probe). This design of probe generally achieves good discrimination in hybridization between different allelic forms.

Indirect detection refers to determining the presence or absence of a specific polymorphism identified in the genetic profile by detecting a surrogate or proxy polymorphism that is in linkage disequilibrium with the SNP in the individual's genetic profile. Detection of a proxy polymorphism is indicative of a polymorphism of interest and is increasingly informative to the extent that the polymorphisms are in linkage disequilibrium, e.g., at least 50%, 60%, 70%, 80%, 90%, 95%, 98%, or about 100% LD. Another indirect method involves detecting allelic variants of proteins accessible in a sample from an individual that are consequent of a risk-associated or protection-associated allele in DNA that alters a codon.

Based on the polymorphisms and associated sequence information disclosed herein, detection reagents can be developed and used to assay any polymorphism of the present invention individually or in combination, and such detection reagents can be readily incorporated into a kit or system. The terms “kits” and “systems,” as used herein in the context of polymorphism detection reagents, are intended to refer to such things as combinations of multiple polymorphism detection reagents, or one or more polymorphism detection reagents in combination with one or more other types of elements or components (e.g., other types of biochemical reagents, containers, packages, substrates, electronic hardware components, etc.). Accordingly, the present invention further provides polymorphism detection kits and systems, including but not limited to, packaged probe and primer, arrays/microarrays of nucleic acid molecules, and beads that contain one or more probes, primers, or other detection reagents for detecting one or more polymorphisms of the present invention. The kits/systems can optionally include various electronic hardware components; for example, arrays (“DNA chips”) and microfluidic systems (“lab-on-a-chip” systems) provided by various manufacturers typically comprise hardware components.

In some embodiments, a polymorphism detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a polymorphism-containing nucleic acid molecule. A kit may further contain means for determining the amount of a target nucleic acid, and means for comparing the amount with a standard, and can comprise instructions for using the kit to detect the polymorphism-containing nucleic acid molecule of interest. In one embodiment of the present invention, kits are provided which contain the necessary reagents to carry out one or more assays to detect one or more polymorphisms disclosed herein. In a preferred embodiment of the present invention, polymorphism detection kits/systems are in the form of nucleic acid arrays, or compartmentalized kits, including microfluidic/lab-on-a-chip systems.

Polymorphism detection kits or systems may contain, for example, one or more probes, or pairs of probes, that hybridize to a nucleic acid molecule at or near each target position. Multiple pairs of allele-specific probes may be included in the kit/system to simultaneously assay large numbers of polymorphisms, at least one of which is a polymorphism of the present invention. In some kits/systems, the allele-specific probes are immobilized to a substrate such as an array or bead. For example, the same substrate can comprise allele-specific probes for detecting any or all of the polymorphisms described herein.

A polymorphism detection kit or system of the present invention may include components that are used to prepare nucleic acids from a test sample for the subsequent amplification and/or detection of a polymorphism-containing nucleic acid molecule. Such sample preparation components can be used to produce nucleic acid extracts (including DNA and/or RNA), proteins or membrane extracts from any biological sample, as described herein.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate by methods known in the art. Any number of probes, such as allele-specific probes, may be implemented in an array, and each probe or pair of probes can hybridize to a different polymorphism position. In the case of polynucleotide probes, they can be synthesized at designated areas (or synthesized separately and then affixed to designated areas) on a substrate using a chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

Another form of kit contemplated by the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other polymorphism detection reagent for detecting one or more polymorphisms of the present invention, one or more containers which contain wash reagents (such as phosphate buffered saline, Tris-buffers, etc.), and one or more containers which contain the reagents used to reveal the presence of the bound probe or other polymorphism detection reagents. The kit can optionally further comprise compartments and/or reagents for, for example, nucleic acid amplification or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art. In such microfluidic devices, the containers may be referred to as, for example, microfluidic “compartments,” “chambers,” or “channels.”

Microfluidic devices and systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more polymorphisms of the present invention. Exemplary microfluidic systems comprise a pattern of microchannels designed onto a glass, silicon, quartz, or plastic wafer included on a microchip. The movements of the samples may be controlled by electric, electroosmotic, or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip.

For genotyping polymorphisms, an exemplary microfluidic system may integrate, for example, nucleic acid amplification, primer extension, capillary electrophoresis, and a detection method such as laser induced fluorescence detection. In a first step of an exemplary process for using such an exemplary system, nucleic acid samples are amplified, preferably by PCR. Then, the amplification products are subjected to automated primer extension reactions using ddNTPs (specific fluorescence for each ddNTP) and the appropriate oligonucleotide primers to carry out primer extension reactions which hybridize just upstream of the targeted polymorphism. Once the extension at the 3′ end is completed, the primers are separated from the unincorporated fluorescent ddNTPs by capillary electrophoresis. The separation medium used in capillary electrophoresis can be, for example, polyacrylamide, polyethyleneglycol or dextran. The incorporated ddNTPs in the single nucleotide primer extension products are identified by laser-induced fluorescence detection.

The present disclosure also provides non-transitory computer-readable media. The non-transitory computer-readable media stores instructions that when executed by one or more processors performs some or all of the operations described in the disclosed methods. In some embodiments, the one or more processors perform operations comprising receiving data identifying the presence or absence of a polymorphism in a biological sample, generating a nonalcoholic fatty acid liver disease risk score from said data, and displaying or reporting said risk score.

The methods described herein can be implemented by one or more processors and a computer-readable medium storing instructions executable by the one or more processors to perform operations, as described above. An at least one computer system may comprise the one or more processors and/or the computer-readable media. The computer system may further comprise one or more local servers or databases connected to or integrated with the at least one computer system. The one or more processors may be configured to communicate via wired or wireless communications with each other or other processors. The one or more processors may be configured to operate on one or more processor-controlled devices that can be similar or different devices.

The readable media described herein may protect the confidentiality and security of protected health information (PHI) in compliance with various privacy standards (e.g., Health Insurance Portability and Accountability Act (HIPAA)). Thus, the readable media may be considered HIPAA-compliant. The readable media and/or the one or more processors may provide or allow one or all of: means of access control, mechanisms to authenticate electronic PHI, functionalities for encryption/decryption, and mechanisms to log activity and implement audits. Data may be communicated using known encryption/decryption and security techniques. For example, DICOM imaging standards support encryption. The system and methods may anonymize any protected subject data.

### EXAMPLES

### Materials and Methods

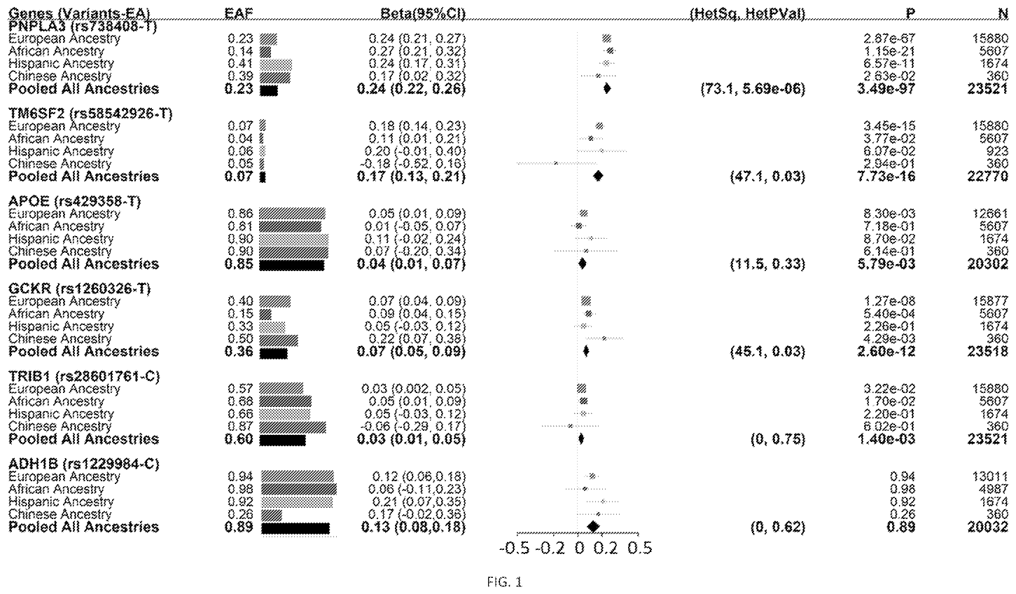

Analyses were carried out in cohorts from the Genetics of Obesity-related Liver Disease (GOLD) Consortium, United Kingdom Biobank (UKBB), FinnGen, Electronic Medical Record and Genomics (eMERGE) Consortium, and Michigan Genomics Initiative (MGI) (FIG. 1).

GOLD Consortium—The multiethnic GOLD Consortium includes nine multiethnic cohorts with CT-measured steatosis (N=23,521): AGES¹¹, COPDGene¹², FamHS¹³, FHS¹⁴, GENOA¹⁵, IRASFS¹⁶, JHS¹⁷, MESA¹⁸, and OOA¹⁹.

UKBB—The UKBB cohort was previously described.²⁰ Participants in the NAFLD analyses were included regardless of ethnicity and excluded if they or their relatives had abdominal MRI images. NAFLD cases were identified by ICD-9 571.8 or ICD-10 K76.0 codes. The UKBB NAFLD dataset included 1,827 NAFLD cases and 436,262 controls. A second UKBB NAFLD European only dataset was assembled as stated above and included 1,706 cases and 412,151 controls.

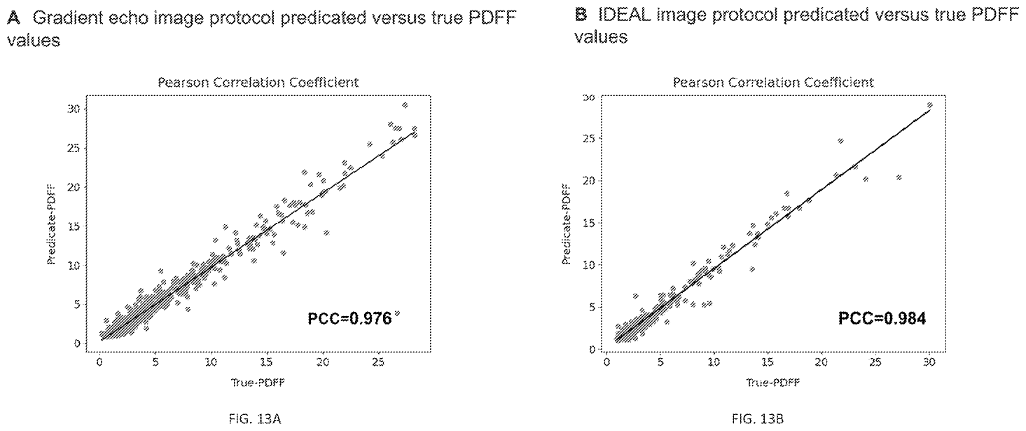

Convolutional neural network (CNN) model for UKBB liver MRI imaging—A CNN model was applied to determine liver proton density fat fraction (PDFF) from MRI in UKBB. UKBB uses two imaging protocols: gradient echo (GRE) (N=10,093) and IDEAL (N=35,779), which includes N=1,491 individuals that had undergone both protocols. To determine the MRI proton density fat fraction (PDFF) for all participants, a standard 2D U-Net was applied to segment the GRE and IDEAL liver data. ITK-SNAP software was used to manually annotate the liver in 98 randomly chosen images from the GRE protocol. Next, the segmented GRE images were split into training (N=64), validation (N=16), and test (N=18) sets. The result showed that liver segmentation achieved Dice scores over 94%. Similarly, the liver was manually annotated in 95 randomly chosen images from the IDEAL protocol. Next, the segmented IDEAL images were split into training (N=64), validation (N=16), and test (N=15) sets. The overall performance of the liver segmentation is also about 94% on Dice scores. After the liver has been identified by 2D U-net model on each slice for all of two imaging protocols, a 2D CNN Residual Neural Network (2D-CNN-ResNet) model using two steps was applied on the segmented liver. From the 4,616 individuals with true PDFF values, quantified by Perspectum Diagnostics from gradient echo imaging, 4,569 individuals with a full set of ten standard liver segmentation images were selected and split into training, validation, and test datasets. The 2D-CNN-ResNet model was trained and validated on 3,500 participants and tested on the remaining 1,069 participants. For the remaining 5,477 individuals from the gradient echo protocol, the CNN model developed here was used to predict PDFF. This 2D-CNN-ResNet model was then applied to estimate the PDFF value of participants from the IDEAL protocol. Based on these overlapping samples (N=1,491) with true PDFF value derived from the first step, 2D-CNN-ResNet model was trained (N=952), validated (N=238), and tested (N=301). PDFF for the remaining 34,351 participants with only IDEAL imaging were then inferred using this CNN model. Inferred PDFF had a Pearson correlation coefficient of 0.976 and 0.984 in the validation and testing datasets. True PDFF values were also measured (FIG. 13). This will be called the UKBB MRI-PDFF dataset, which after accounting for genetic missingness (N=1,151) totaled N=43,293. A second UKBB MRI-PDFF dataset included only European participants and totaled N=41,834.

eMERGE—The eMERGE NAFLD cohort (N=1,106 cases; 8,571 controls) was previously described and summary statistics are available at ebi.ac.uk/gwas/studies/GCST008468. Effect allele frequencies were not available and were estimated using UK Biobank Europeans.

FinnGen—FinnGen data freeze 4 summary statistics from finngen.fi/fi (N=651 NAFLD cases, 176,248 controls) was used for the analysis described herein.

MGI—MGI is a hospital-based cohort of patients seen at Michigan Medicine (Ann Arbor, MI). The MGI cohort was previously described.²³ NAFLD cases were identified by ICD-9 571.8, or ICD-10 K76.0, and HCC by ICD-9 155.0 or ICD-10 C22.0. Cirrhosis was defined by ICD-9 571.2 or 571.5 or 571.6, or ICD-10 K70.2-4 or K74.x or K71.7 or NLP (which has been previously described).23

Genome-wide association study (GWAS) and meta-analysis—GWAS of autosomal variants was carried out assuming additive effects in each of the nine GOLD cohorts separately. The analyses were corrected for age, age², sex, alcoholic drinks, and principal components (PCs) or admixture. Sensitivity analyses by sex, study, and ancestry did not show significant heterogeneity allowing us to combine the data across cohorts for all individuals with genetic data (N=23,521). The GOLD Consortium meta-analysis was performed using the inverse variance approach in METAL (08/28/2018 release).

GWAS of autosomal variants were carried out independently in UKBB using linear mixed modeling using SAIGE (version 0.29) with binary NAFLD or inverse normally-transformed MRI-PDFF as the dependent variable using an additive genetic model. A SNP imputation quality cutoff of 0.85 was used. The model was controlled for sex, age, age², and PCs 1-10.

Summary statistics from FinnGen and eMERGE studies were combined with the UKBB NAFLD, UKBB MRI-PDFF, and GOLD CT steatosis analyses using a sample size and direction of effect meta-analysis implemented in METAL (FIG. 1) in an analysis referred to herein as GOLDPlus. Multi-allelic variants, indels, variants with minor allele frequency<0.001, and variants with minor allele count<400 were excluded. Variants with HetP-value<0.05 and opposing directionality were also excluded across studies. A p-value<5.0×10⁻³ was considered genome-wide significant. Given the multiethnic nature of the analysis, independent loci were identified using a 500Kb flanking criteria from the lowest p-value associated variant. To ascertain independent signals, a direct conditional analysis was also performed for all top hits using the UKBB multiethnic cohort. To perform conditional analysis, the genetic dosage of the loci was added to the other covariates (age, age², sex, PCs 1-10) of SAIGE step 1 and the GWAS was rerun.

Ancestry-specific and sex-specific analyses in the GOLD Consortium—In order to assess ancestry-specific differences, a meta-analysis was conducted in the GOLD Consortium for each ancestry (European, African, Hispanic, and Chinese) separately and all ancestries together using METAL. Additionally, separate GWAS in men and women in the GOLD Consortium were conducted and meta-analyzed the GWAS using METAL. Sex-specific GWAS analyses were controlled for age, age², and PCs 1-10. Cochran's Q was used to assess the observed heterogeneity and the I² metric was used for quantification. A Cochran's Q p-value<2.0×10⁻⁴ was considered significant.

GWAS analysis stratified by alcohol use—Using the UKBB MRI-PDFF data alcohol-specific GWAS of heavy and light drinkers was performed. Heavy drinkers were identified as ≥14 drinks consumed per week for males or ≥7 drinks a week for females (N=21,396) and light drinkers as ≤1 drinks consumed per week for males and females (N=9,888). The UKBB MRI-PDFF GWAS were carried out as described above. A meta-analysis of the heavy and light drinkers was performed using METAL in order to assess the heterogeneity.

Previously published NAFLD/Steatosis variants—The effects of previously reported NAFLD/Steatosis variants were evaluated in GOLDPlus. A literature search was conducted for NAFLD and steatosis GWAS in PubMed and genome-wide significant variants were identified. Variants that were independent of the GOLDPlus genome-wide significant variants (500Kb flanking criteria from the lowest p-value associated variant) were assessed.

Phenome-wide association study (PheWAS)—Publicly available UKBB GWAS data from the Neale lab was utilized to perform a PheWAS of the NAFLD increasing alleles with related phenotypes. Associations were considered significant with a p-value<0.05.

PheWAS clustering—The PheWAS data was clustered by Z-score for the respective phenotype/variant combinations. Clustering was performed using R version 4.0.2. Optimal clusters were determined using the ‘NbClust’ package version 3.0. The ‘stats’ package was used for K-means clustering and the ‘dendextend’ version 1.13.4 and ‘dendogram’ packages were used for hierarchical clustering.

Mendelian randomization—A two-sample Mendelian randomization (MR) was performed, implemented in R version 3.6.0 using ‘TwoSampleMR’ version 0.5.5. For the analysis, the variant-NAFLD effect estimates from the GOLD Consortium (betas are required for MR and the GOLD Consortium data had the highest quality measures of hepatic steatosis in the population-based cohorts) were used. Only those variants with an F-statistic>10 were included in the MR analysis. 43 MR was performed using the resulting variants as the exposure and related publicly available and UKBB GWAS (K74 fibrosis and cirrhosis of liver and 185 oesophageal varices, a complication of cirrhosis) as outcomes. The reverse analysis was also performed where independent genome-wide significant (p-value<5.0×10⁻⁸) variants from the aforementioned GWAS were used as exposure and the GOLD Consortium phenotype as the outcome. Inverse-variance weighted, penalized weighted median, weighted median, weighted mode, and MR-Egger methods were also applied. Tests for heterogeneity and horizontal pleiotropy were also performed.

Data-driven expression prioritization integration for complex traits (DEPICT)—DEPICT provides details regarding GWAS-prioritized tissues, genes, and pathways across cells and tissues.⁴⁴ Enrichment was considered statistically significant at a false discovery rate (FDR) p-value<0.05.

Polygenic risk scores (PRS) and NAFLD risk factors—A PRS was created using the liver fat increasing variants (N=17) from the GOLDPlus meta-analysis. The PRS was based on a weighted sum of dosage of the NAFLD associated single variants. The beta value of each allele (from GOLD Consortium) was used to weigh the PRS. The predictive power of the PRS was assessed on NAFLD, cirrhosis, and HCC cohorts in MGI European ancestry samples. PRS were defined as inverse-normally transformed rank units or as percentiles. Analyses were adjusted for age, age², sex, and PCs 1-10. The predictive power of the PRS was assessed in comparison to other NAFLD risk factors using univariate and multivariate linear models. NAFLD risk factors were the median outpatient values for the MGI cohort. Linear models were generated using the ‘glm’ function in R. The C-statistic was calculated using the ‘DecsTools’ package in R.

### Example 1

GOLDPlus meta-analysis

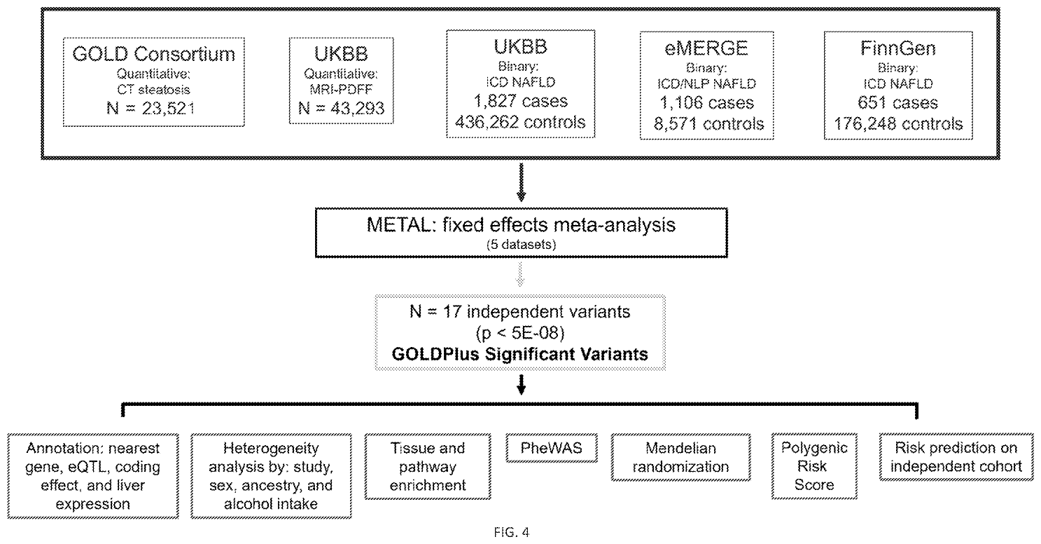

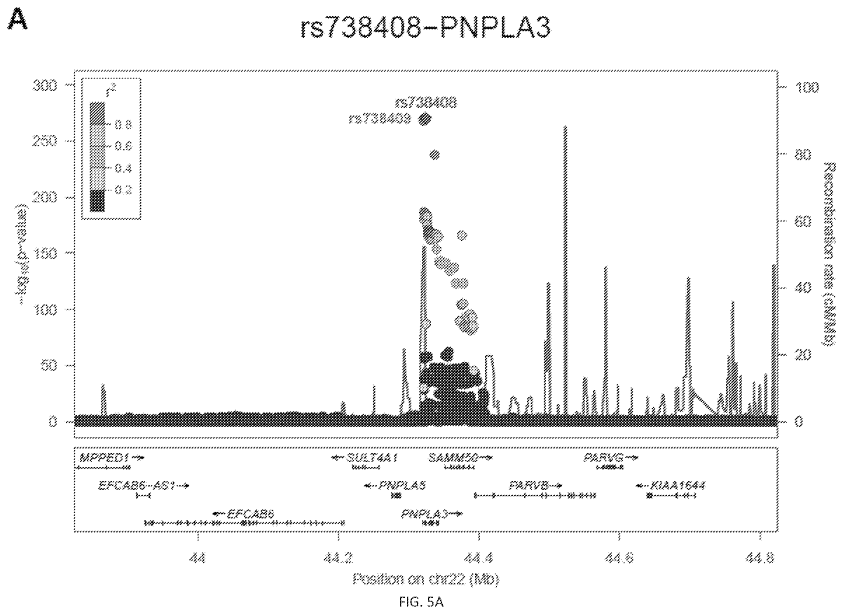

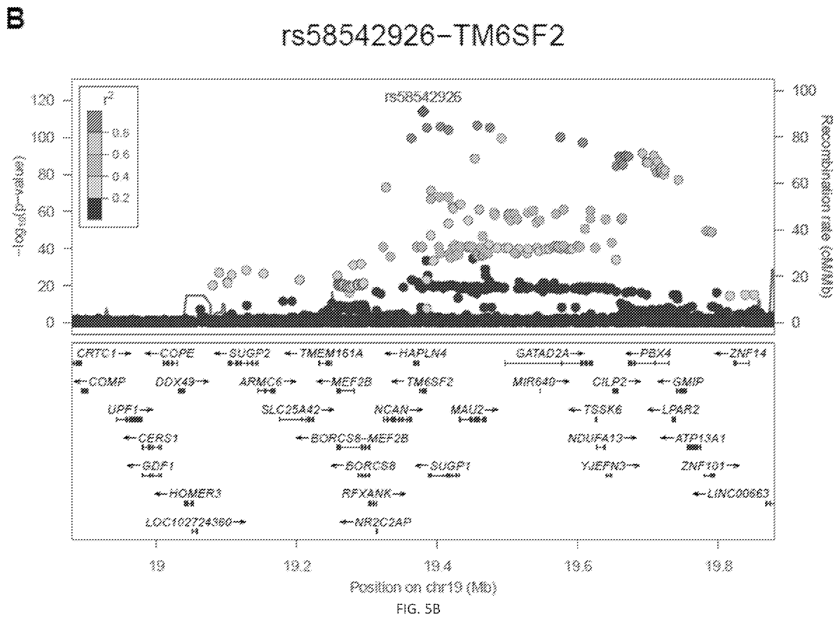

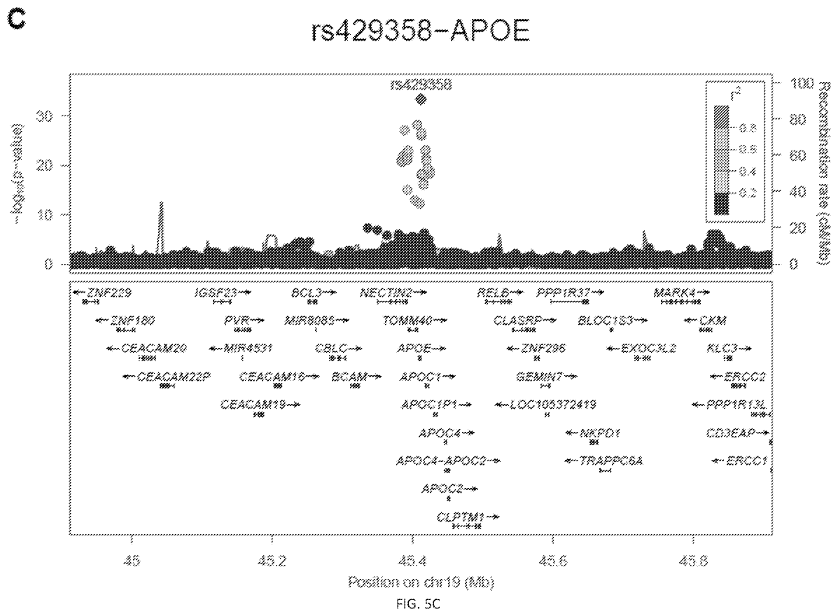

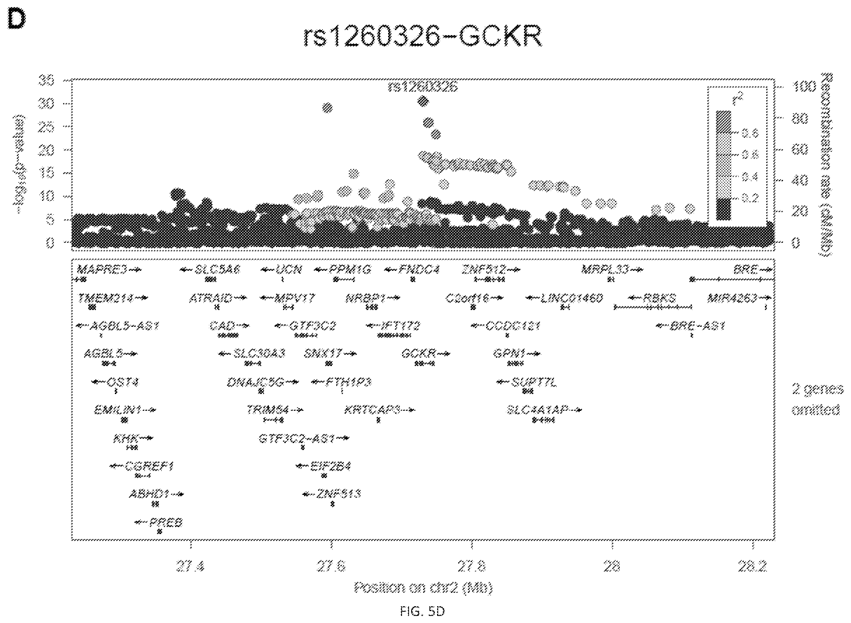

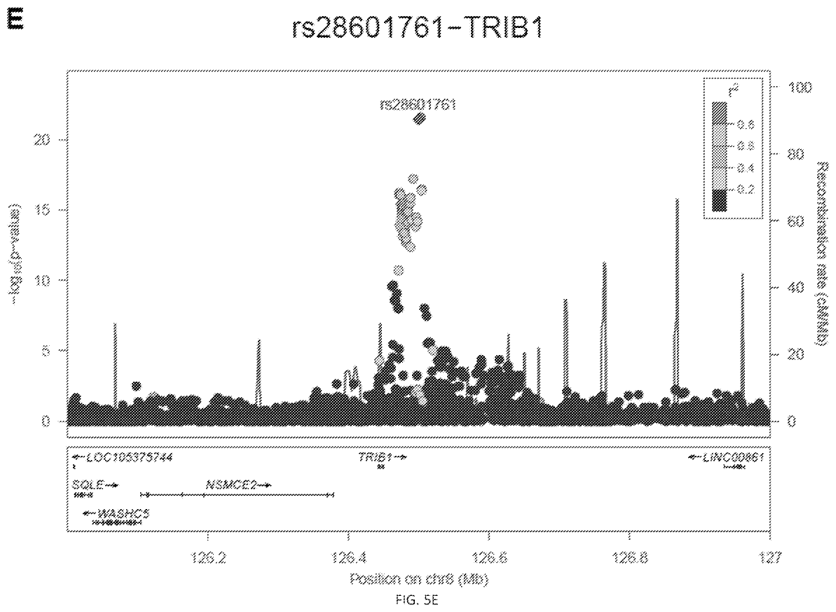

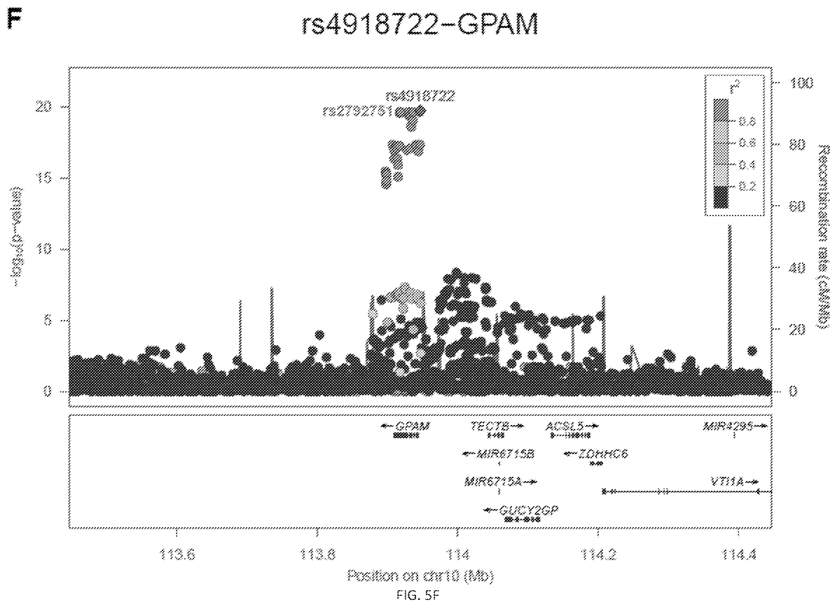

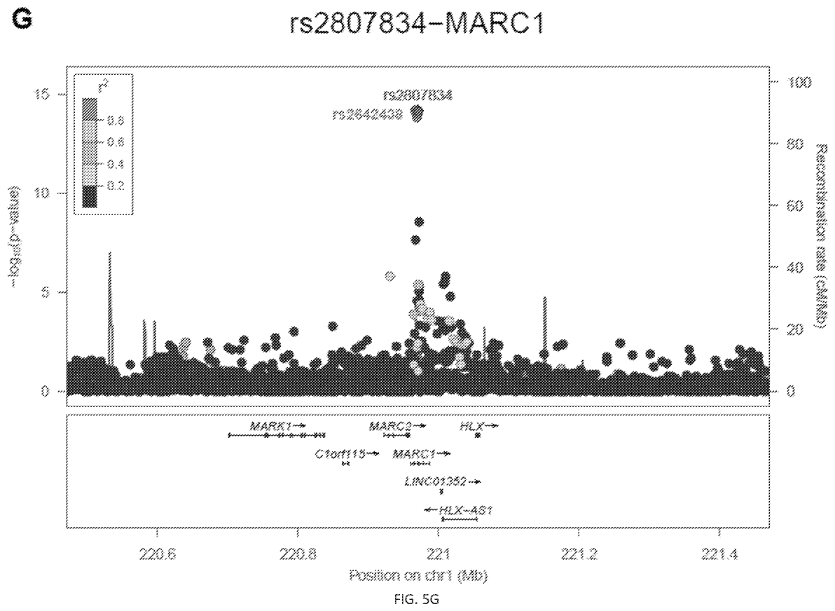

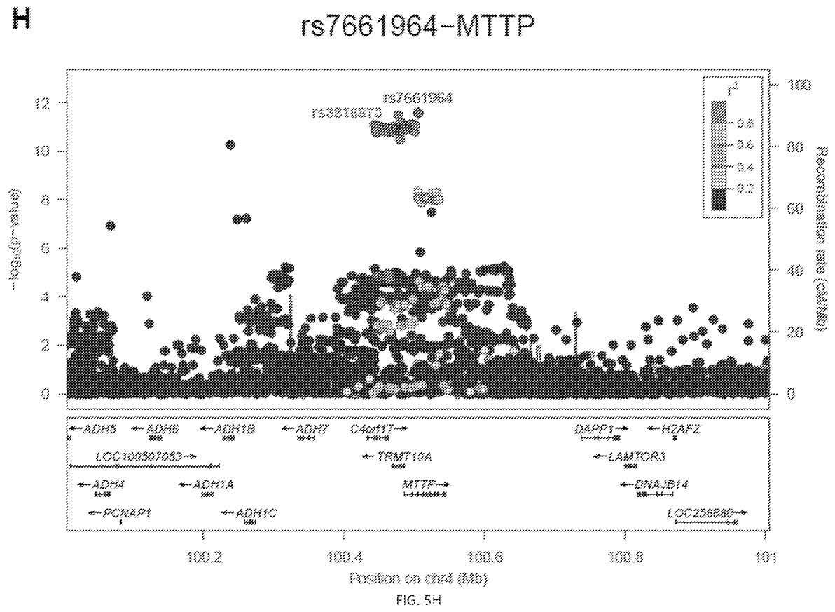

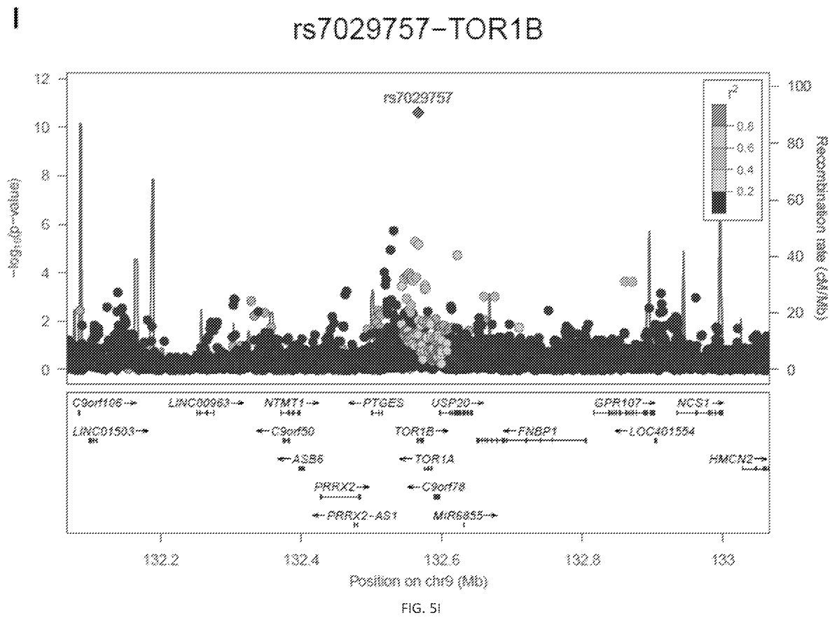

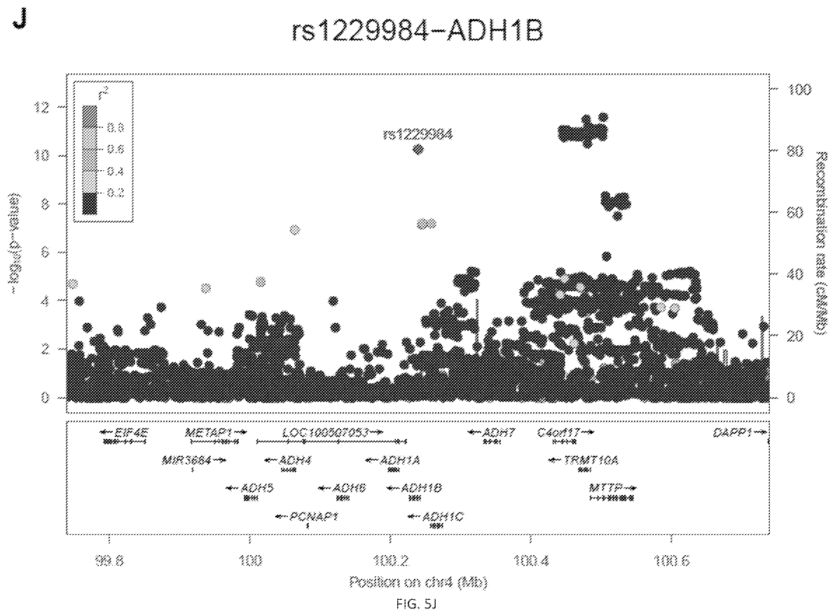

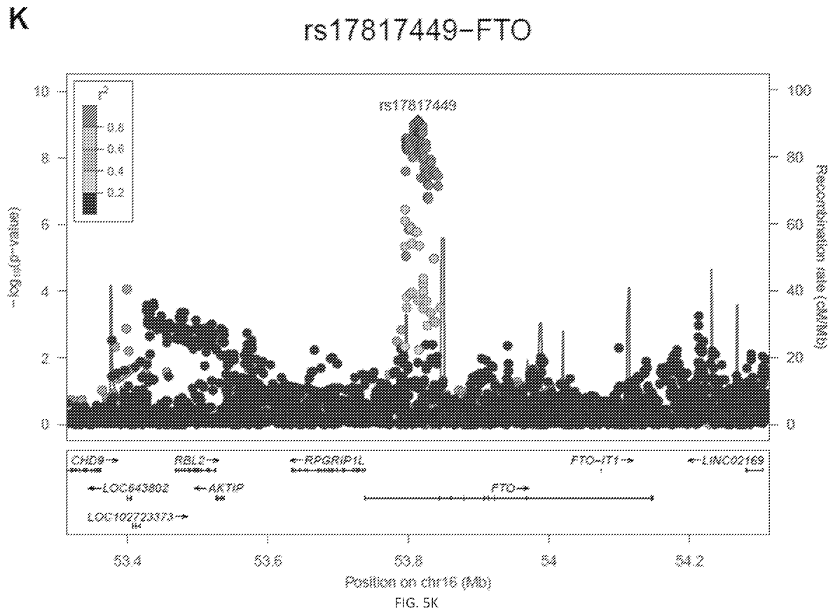

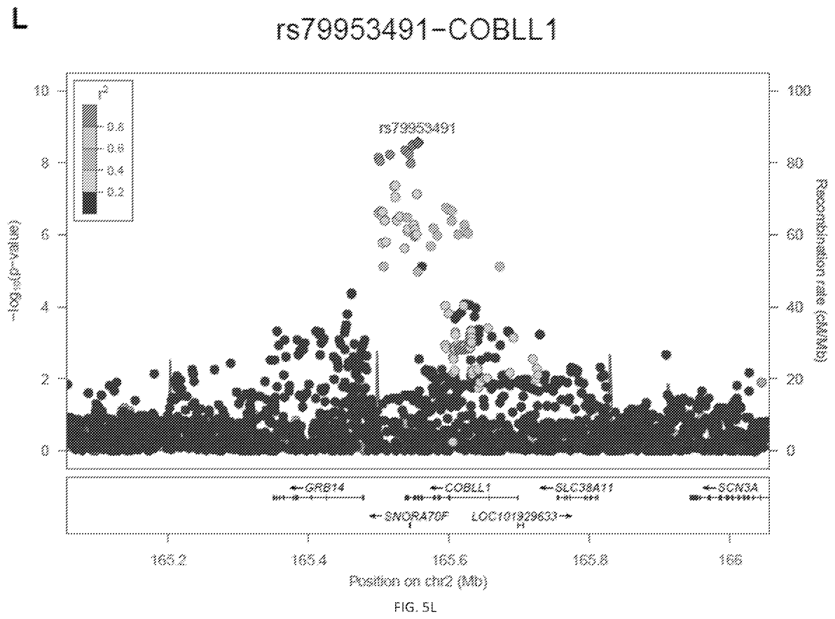

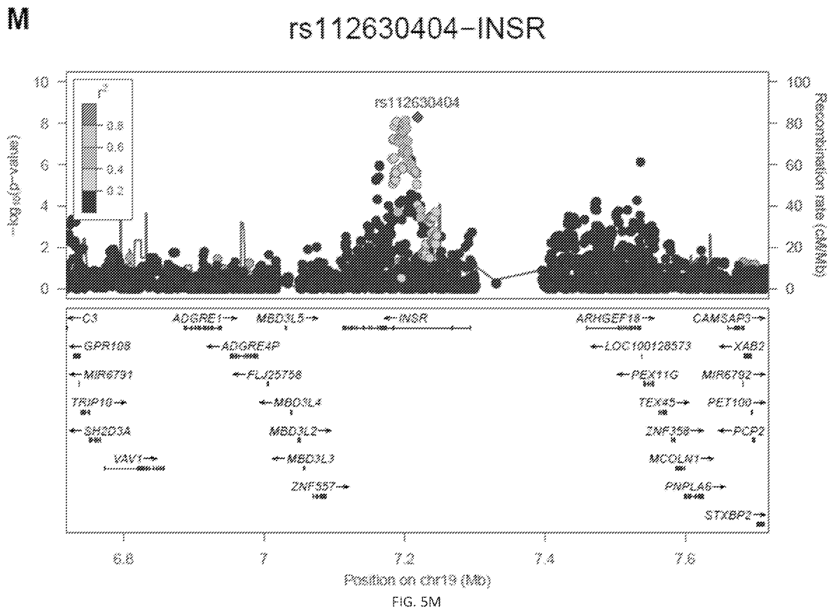

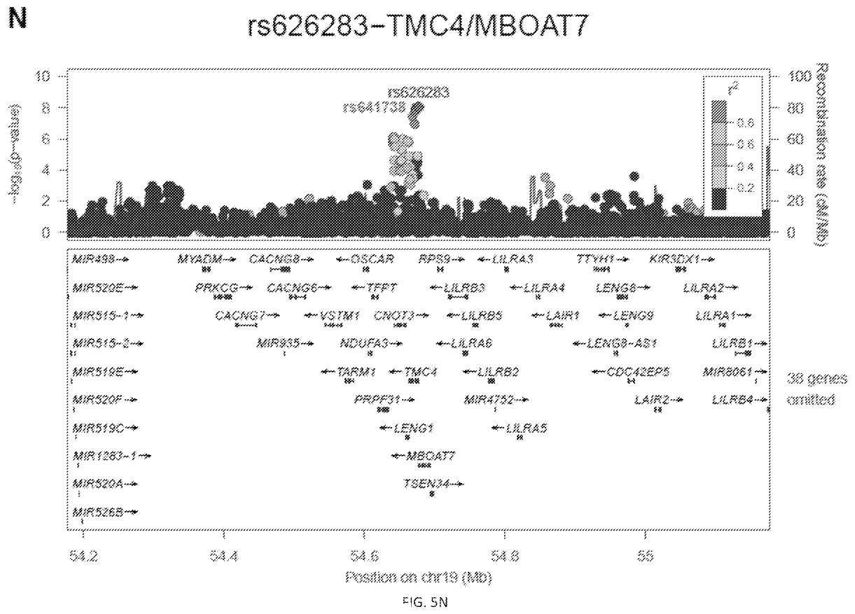

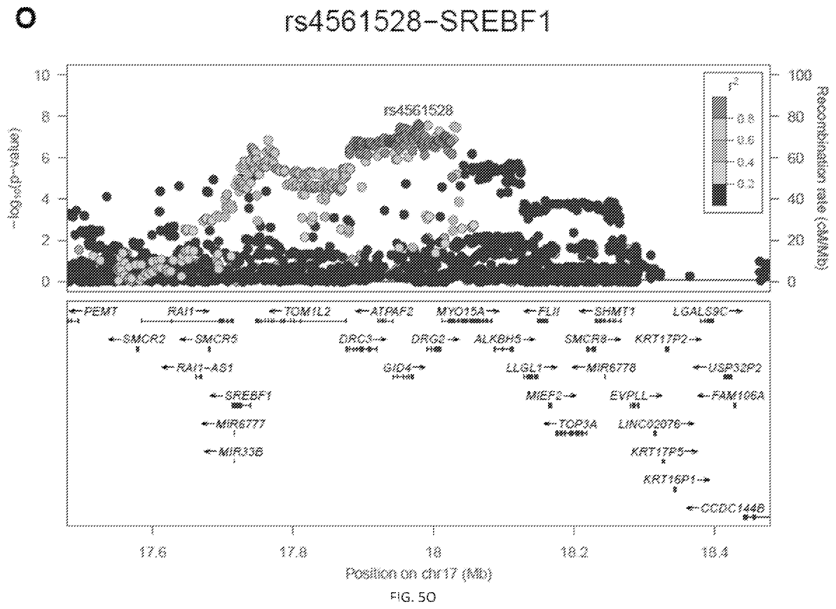

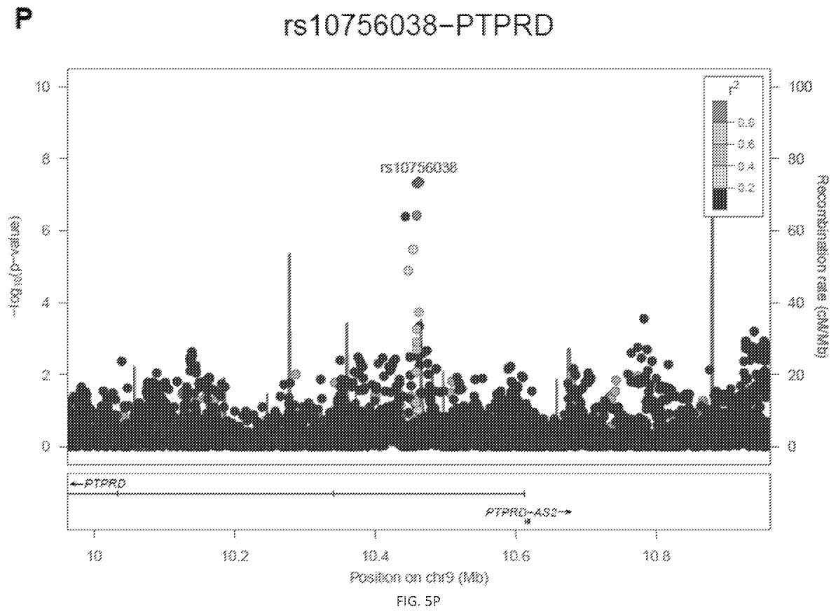

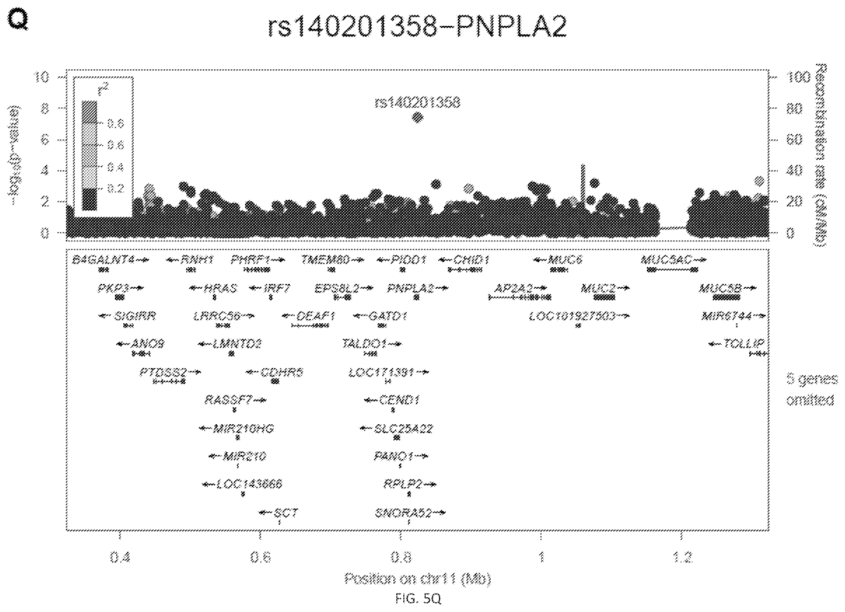

A meta-analysis of CT measured liver fat (GOLD) was carried out with UKBB MRI liver PDFF, UKBB NAFLD, eMERGE NAFLD, and FinnGen NAFLD in the largest meta-analysis to date of NAFLD (FIG. 4). In all cases the top associated variants for all datasets were at PNPLA3 verifying congruency across the phenotypes. Eleven independent genome-wide significant variants were identified (p-value<5.0×10⁻⁸) (Table1; FIG. 5). These variants are referred to as the GOLDPlus Significant Variants. Genes for annotation were prioritized if the index variant was a missense variant in the gene, in high LD (r²>0.7) with an exonic variant in the gene, and/or was an eQTL for the gene in liver. Genes that were within 1 Mb of the index variant and predominantly expressed in the liver, prioritized by DEPICT analysis, and/or nearest to the index variant were also prioritized for annotation.

One region contained possible two independent loci within close proximity of each other: one at ADH1B—rs1229984 which is within 500 kb of MTTP-rs7661964. To confirm that these two signals were independent of each other conditional analyses were carried out in the UKBB multiethnic dataset. ADH1B in the UKBB multiethnic cohort had a p-value=5.09E-06 and a p-value=1.03E-05 before and after conditioning on MTTP. MTTP had a p-value=2.01E-07 and a p-value=4.09E-07 before and after conditioning on ADH1B. Novel variants were defined as those more than 1 MB away from genome-wide significant variants (p-value<5.0×10⁻⁸) from previously published NAFLD and hepatic steatosis GWAS. Novel associations were identified in or near TOR1B, FTO, COBLL1/GRB14, INSR, SREBF1, and PNPLA2 (Table1; FIG. 5). Previously identified NAFLD associations were confirmed in or near PNPLA3, TM6SF2, APOE, GCKR, TRIB1, GPAM, MARC1, MTTP, ADH1B, TMC4/MBOAT7, and PTPRD. One genome-wide significant variant LOC157273/PPPIR3B (rs4841132; p-value=4.21λ10⁻¹³; HetP-value=7.44×10⁻¹⁹) was removed from downstream analysis due to phenotype heterogeneity (see Methods). rs4841132 is known to promote liver damage by increasing glycogen, which is a distinct pathology from NAFLD.

The index variants at several loci are missense variants: TM6SF2, APOE, GCKR, ADH1B, and PNPLA2. The index variants in PNPLA3, GPAM, MARC1, MTTP, and TMC4/MBOAT7 are in LD (r²>0.99 across all ethnicities) with missense variants PNPLA3 (1148M; rs738409), GPAM (V43I; rs2792751), MARC1 (T493A; rs2807834), MTTP (145T; rs3816873), and TMC4/MBOAT7 (TMC4 G17E; rs641738) respectively. The index variants associated with TRIB1 and SREBF1 are intergenic, while the variants in TOR1B, FTO, COBLL1/GRB14, INSR, and PTPRD are intronic. rs7029757 is an eQTL for TOR1B (FDR p-value=5.00E-04), which is expressed in the liver. TRIB1, MTTP, TOR1B, INSR and PTPRD are the genes nearest to the respective non-coding index variants. SREBF1 is within 1 MB of the index variant and is highly expressed in the liver. rs79953491 is an intronic variant in COBLL1 which is expressed in the liver. Additionally, GRB14, which is highly expressed in the liver, is within 1 MB of rs79953491. Literature review suggests that rs56094641 at FTO may exert its effects on BMI by affecting IRX3/6 expression in adipose tissue.

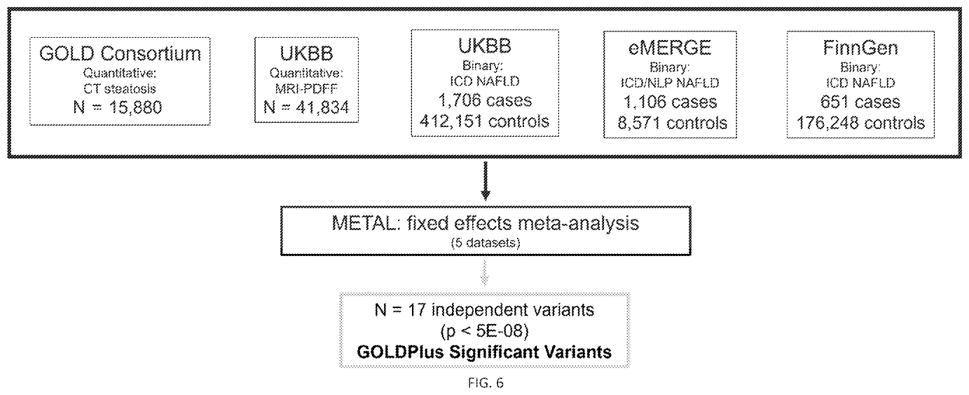

A second meta-analysis was performed using the same datasets but included only European ancestry participants (FIG. 6). Seventeen independent genome-wide significant variants were also identified (p-value<5.0×10⁻⁸) (PNPLA3, TM6SF2, APOE, GCKR, TRIB1, GPAM, MARC1, MTTP, ADH1B, TOR1B, TMC4/MBOAT7, COBLL1/GRB14, SREBF1, INSR, FTO, PNPLA2 and TAMM41/SYN2) (Table 2). The European meta-analysis differs only at one locus from the multiethnic analysis: TAMM41/SYN2 is genome wide significant in the European analysis whereas PTPRD is significant in the multiethnic analysis. The overlapping genome-wide significant variants shared across the two analyses have a less significant p value of association in the European data due to the smaller sample size in this dataset.

### Example 2

### Effects of Identified Variants by Study, Ancestry, Sex, and Alcohol Intake

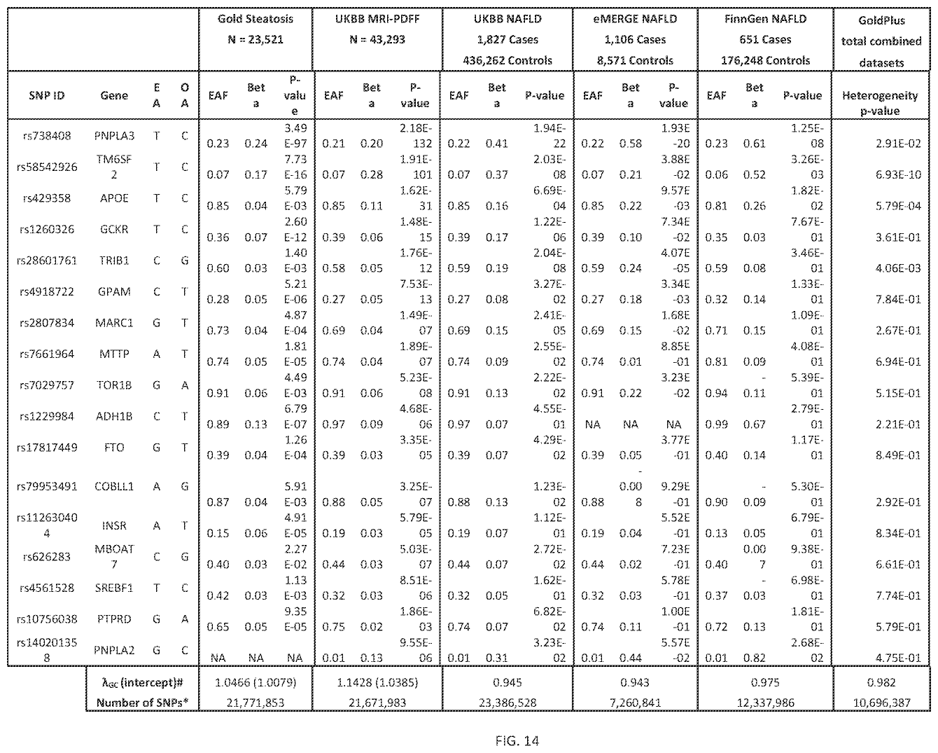

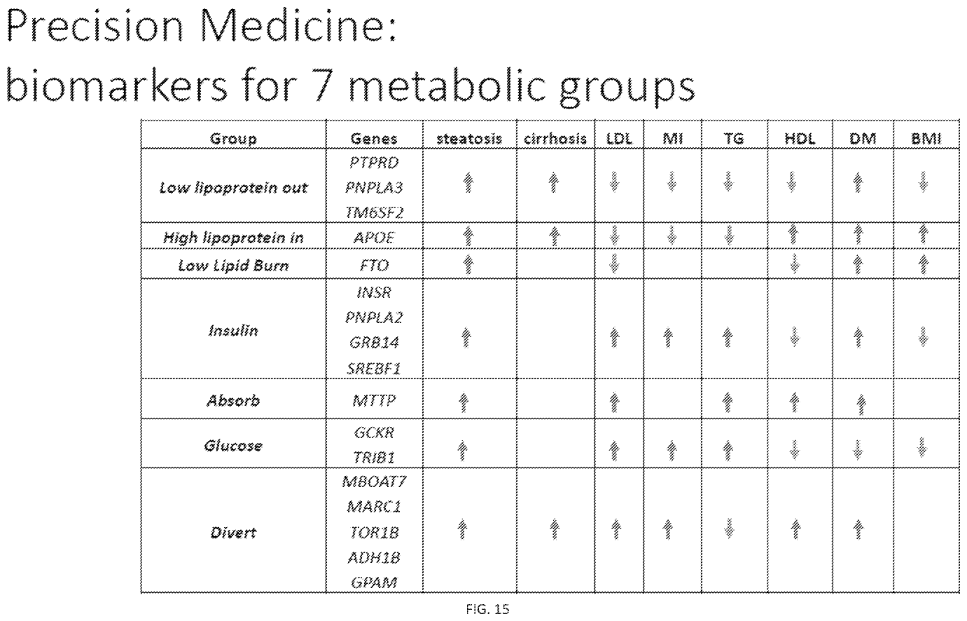

The heterogeneity of effect of the NAFLD associated variants across the studies was assessed in GOLDPlus. After Bonferroni correction, only s58542926 at TM6SF2 and rs429358 at APOE showed statistically significant heterogeneity of effect. However, its direction of effect across studies was congruent. For completeness, the effects of the loci overall are shown and stratified by cohort (Table 1 and FIG. 14, respectively).

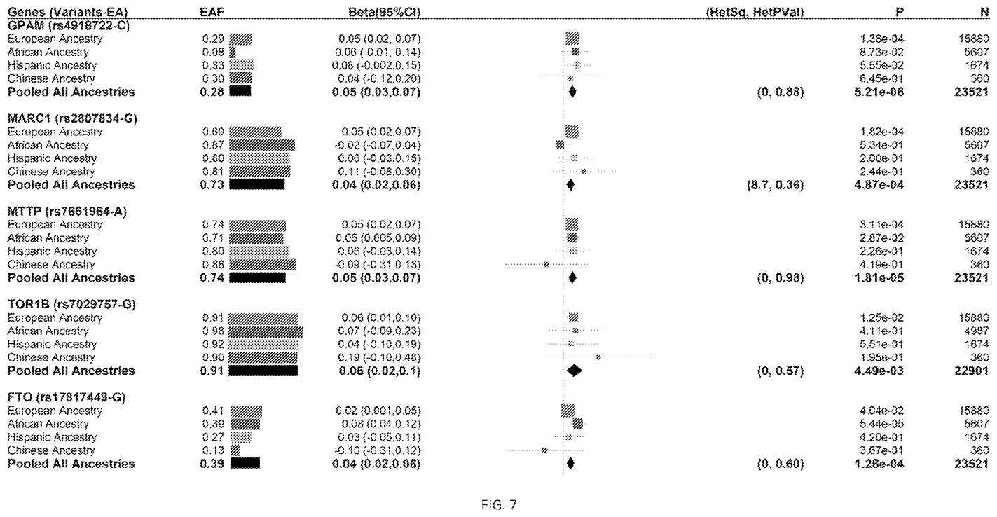

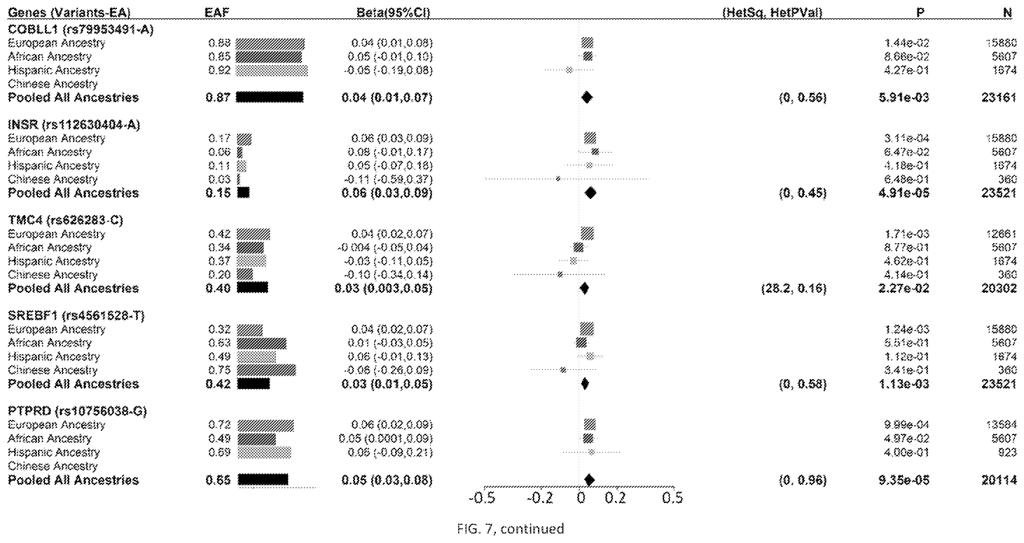

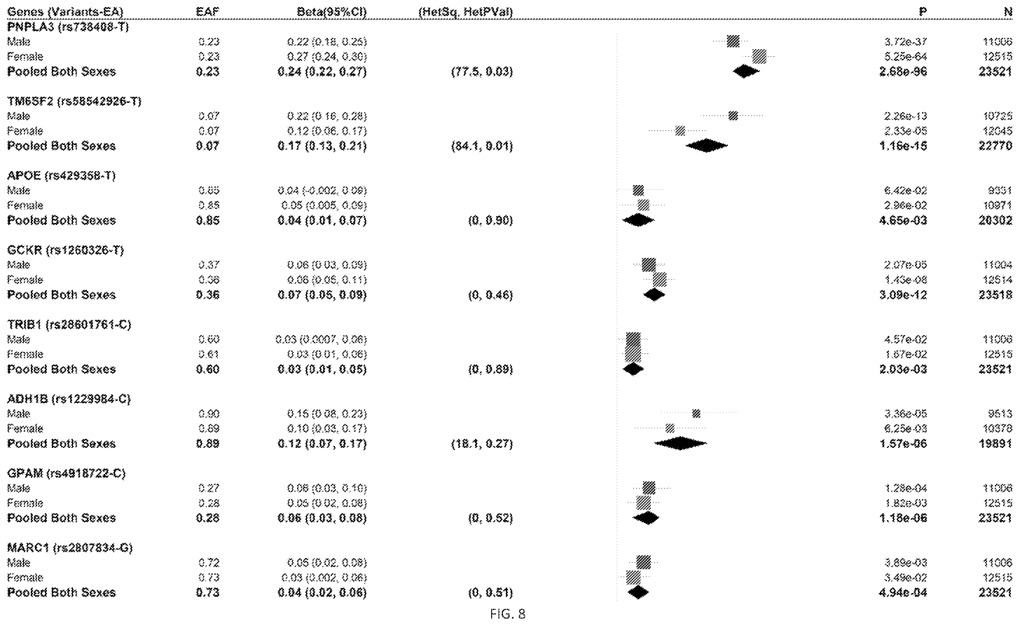

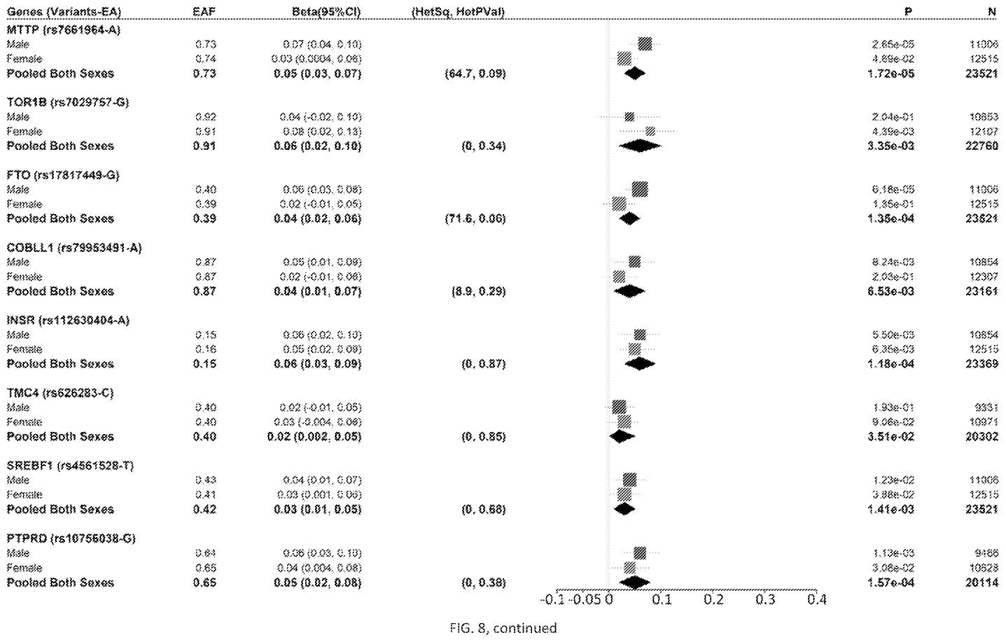

The effects of the NAFLD associated variants across ancestries were assessed (FIGS. 1 and 7) (European (EUR), N=15,880; African (AFR), N=5,607; Hispanic (HIS), N=1,674; and Chinese (CHN), N=360) and sex (males, N=11,006; females, N=12,515) (FIG. 8). For these analyses, the GOLD Consortium data was utilized, which had the highest quality measures of hepatic steatosis in population-based cohorts across ancestries and sex. PNPLA3 (B=0.24 EUR, B=0.27 AFR, B=0.24 HIS, B=0.17 CHN, HetP-value=5.69×10⁻⁶) exhibited significant heterogeneity of effect across ancestries. However, a limited sample size in the Chinese ancestry cohort likely caused unstable estimate of betas, influencing the estimates of heterogeneity. After removal of the Chinese cohort from the meta-analysis the heterogeneity P-value was non-significant after Bonferroni correction (PNPLA3, HetP-value=0.69). No other loci showed significant heterogeneity of effect by ancestry or sex.

Greater than a 10% absolute difference in effect allele frequencies (EAF) was found for index variants in PNPLA3 (rs738408-T), GCKR (rs1260326-T), TRIB1 (rs28601761-C), GPAM (rs2792735-G), MARC1 (rs2642438-G), ADH1B (rs1229984-C), FTO (rs62033399-T), PTPRD (rs10756038-G), TMC4/MBOAT7 (rs641738-T), MAST3 (rs273507-C), ERLIN1 (rs17729876-G), OSGIN1 (rs4782568-C), COBLL1 (rs6712203-C), ITPR2 (rs10842708-G), SDCBP (rs113895159-C), and SUOX (rs705699-G) across ancestries (FIGS. 1 and 7). Variants in six genes, PNPLA3, GCKR, GPAM, PTPRD, COBLL1/GRB14, and INSR, had a relative decreased frequency of the NAFLD increasing allele while those in TRIB1, MARC1, and SREBF1 had an increased frequency in the African ancestry cohort as compared to the European ancestry cohort. In the Hispanic cohort, as compared to the European cohort, the frequency of the NAFLD increasing allele was lower in variants in GCKR and FTO and higher in PNPLA3, TRIB1, MARC1, COBBL1/GRB14, and SREBF1. In the Chinese cohort, as compared to the European cohort, the frequency of the NAFLD increasing allele was lower in variants in ADH1B, FTO, INSR, and TMC4/MBOAT7 and higher in PNPLA3, GCKR, TRIB1, MARC1, MTTP, COBLL1/GRB14, and SREBF1. PNPLA2 is a rare variant and was not well imputed in GOLD Consortium datasets and thus QC′d out.

The starkest contrasts in allele frequencies across ancestries existed in ADH1B. In the Chinese ancestry cohort ADH1B (rs1229984-C) had an EAF of 0.26, while it had >65% EAF in the European, African, and Hispanic ancestry cohorts. The variance explained across the ancestries paralleled the allele frequencies more than the effect sizes, which were similar across ancestries. The highest variances explained were 2.79% in the Hispanic cohort for PNPLA3, 2.42% in the Chinese cohort for GCKR, and 2.04% in the European cohort for PNPLA3. Taken together, these findings suggest EAF, more than effect size, accounts for the differences in genetic disease burden across ancestries. To assess the effects of alcohol the largest population based cohort, UKBB MRI-PDFF, was used to perform a GWAS analysis stratified by alcohol use. After Bonferroni correction, only ADH1B exhibited significant heterogeneity of effect (HetP-value=6.16E-04) between heavy (>14 drinks per week for males or >7 drinks a week for females; N=21,396) and light (≤1 drinks per week for males and females; N=9,888) drinkers for the NAFLD associated variants. ADH1B had a significantly greater effect (B=0.20) in heavy drinkers as compared to light drinkers (B=0.03).

### Example 3

### Tissue, Gene-Set, and Pathway Analyses

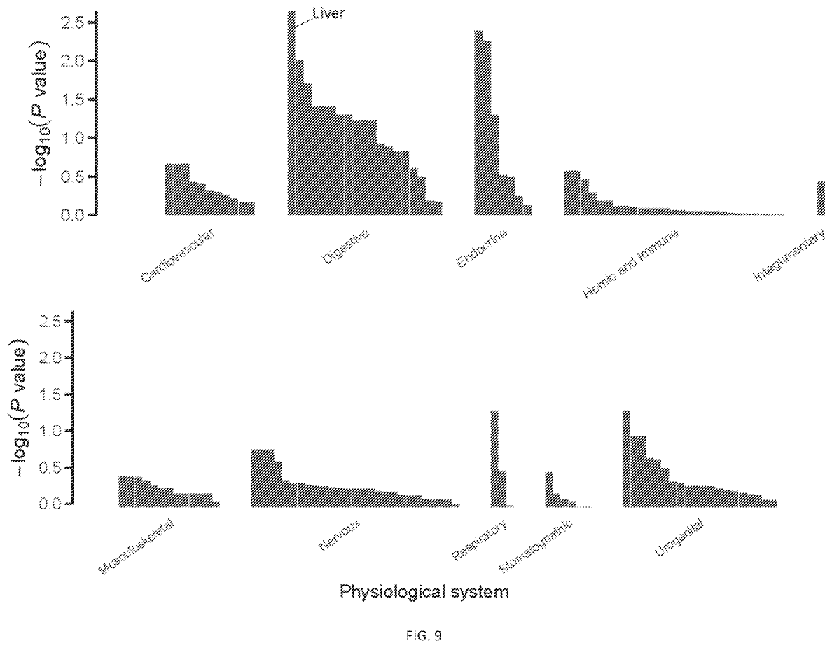

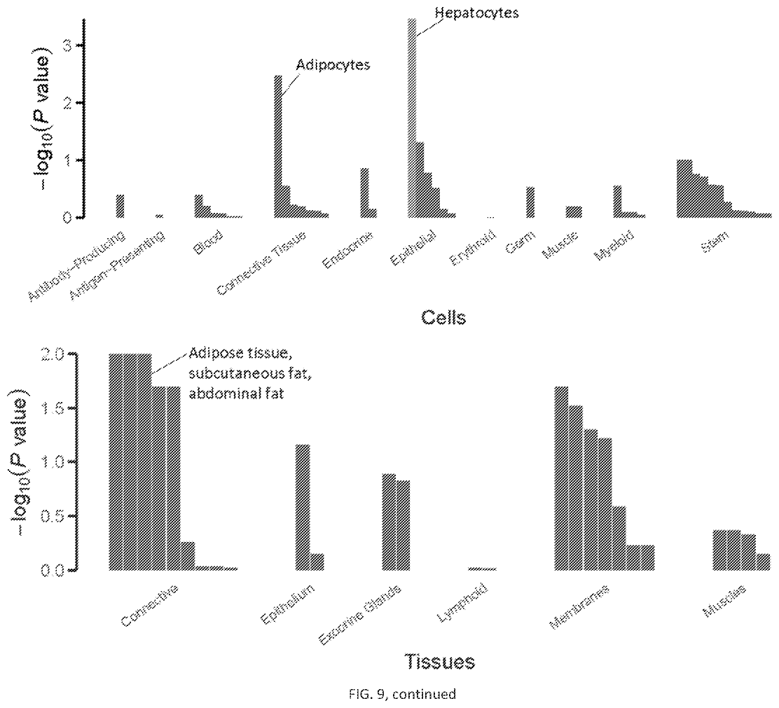

To further understand the biology underlying NAFLD associations, DEPICT was used to identify enriched tissues and cell types (FDR p-value<0.05).44 Input into DEPICT included the 17 NAFLD associated single variants. Liver and adipose tissue were the most enriched tissue types (FIG. 9). Epithelial cells (hepatocytes) were the most enriched cell type (FIG. 9). Using mSigDB significant gene functional overlaps were computed. Enrichment was found (FDR p-value<0.01) in the following biological functions: lipid homeostasis, lipid metabolic processes, monocarboxylic acid metabolic processes, alcohol metabolic processes, lipid biosynthesis, regulation of cholesterol biosynthesis, and steroid biosynthesis.

### Example 4

Association of NAFLD Variants with Other Phenotypes

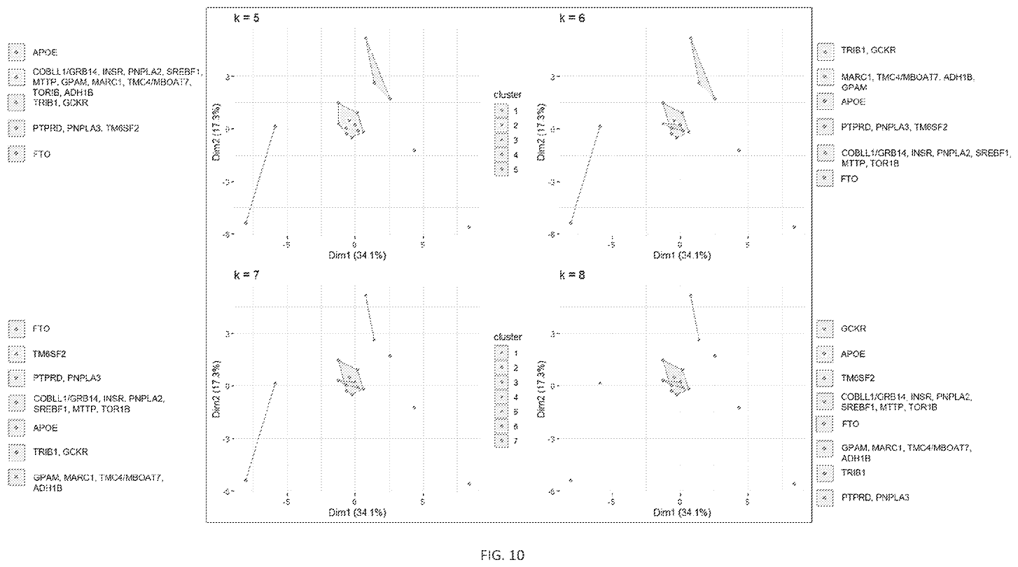

Publicly available GWAS data was utilized to perform a PheWAS of NAFLD-risk increasing alleles with ICD-based diseases; alcohol intake; cardiovascular and body composition measures; and lipid, metabolic, and liver function test blood values (FIG. 2). Clustering of the PheWAS results revealed six distinct groups with differing biological effects (FIG. 10). The NAFLD-risk increasing allele of the variants broadly separated into two groups: one showing significant associations with increased serum low density lipoprotein cholesterol (LDL) and increased alanine aminotransferase (ALT) (TRIB1, GCKR, COBLL1/GRB14, INSR, PNPLA2, SREBF1, MTTP, GPAM, MARC1, TMC4/MBOAT7, TOR1B, and ADH1B associations) and the other group exhibiting decreased associations with LDL and increased associations with ALT (FTO, PTPRD, PNPLA3, TM6SF2, and APOE). Further separations showed NAFLD associating variants at TRIB1, GCKR, COBLL1/GRB14, INSR, PNPLA2, and SREBF1 were distinguished from TOR1B, MARC1, GPAM, TMC4/MBOAT7, and ADH1B associations by being associated with high serum triglycerides and low high-density lipoprotein (HDL) cholesterol. NAFLD associated variants at TRIB1 and GCKR were distinguished from COBLL1/GRB14, INSR, PNPLA2, SREBF1, and MTTP, SREBF1 by being associated with low risk of cholelithiasis and cholecystitis; GCKR had particularly strong association with lower insulin-like growth factor 1 (IGF1) and sex hormone binding globulin (SHBG) levels. NAFLD increasing associations at PTPRD, and FTO all associated with increased serum triglycerides whereas those at PNPLA3, TM6SF2, and APOE associated with decreased serum triglycerides. FTO clustered alone, and differed from other loci in having very strong association with increased body mass index (BMI). Likewise, APOE clustered alone and differed from PNPLA3, and TM6SF2 associations in having an increased association with body composition measures and decreased association with familial Alzheimer's disease.

### Example 5

### Mendelian Randomization

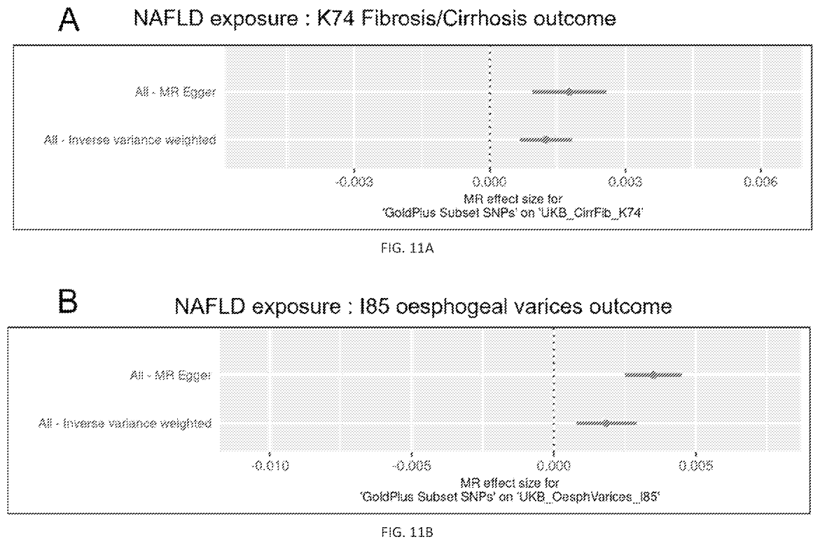

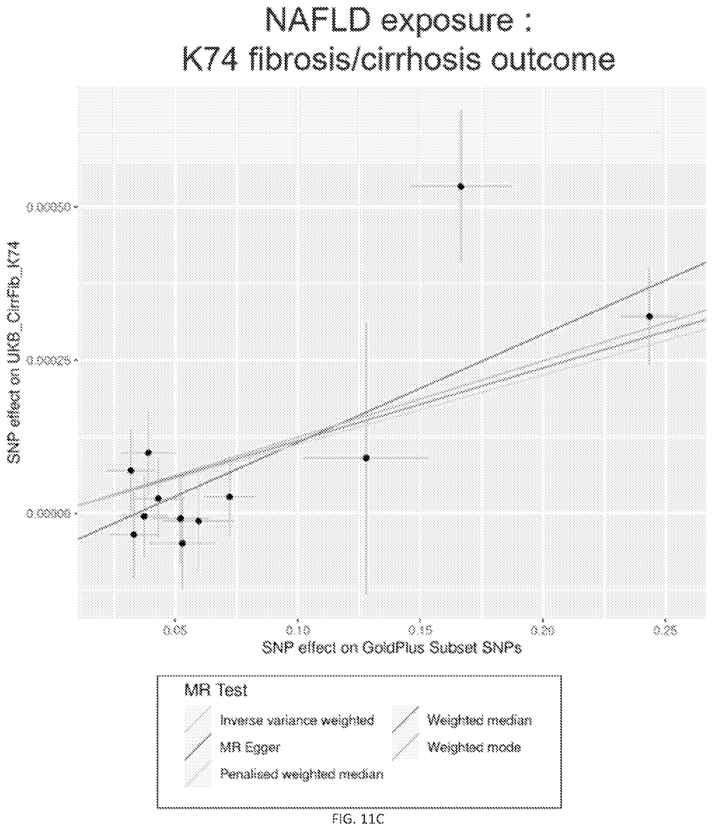

To determine whether NAFLD causally influences liver and metabolic diseases and traits two-sample Mendelian randomization was performed. NAFLD associated variants with an F-statistic>10 were used as a combined instrumental variable for steatosis (N=12; combined F-statistic=158.2). Using the GOLD Consortium effects as the exposure, NAFLD increased risk of liver fibrosis and cirrhosis (ICD K74; OR=1.002, 95% CI=1.001-1.003, MR-Egger p-value=1.69E-03) and esophageal varices (ICD 185; OR=1.003, 95% CI=1.002-1.004, MR-Egger p-value=1.75E-04) (FIG. 11). The MR Egger heterogeneity p-values were non-significant for fibrosis (p-value=0.21) and esophageal varices (p-value=0.08). The MR Egger pleiotropy p-values were non-significant for fibrosis (p-value=0.19) but were significant for esophageal varices (p-value=0.02), indicating horizontal pleiotropy may be driving the results of the esophageal varices Mendelian randomization. Sensitivity analyses are shown in FIGS. 11C-11D.

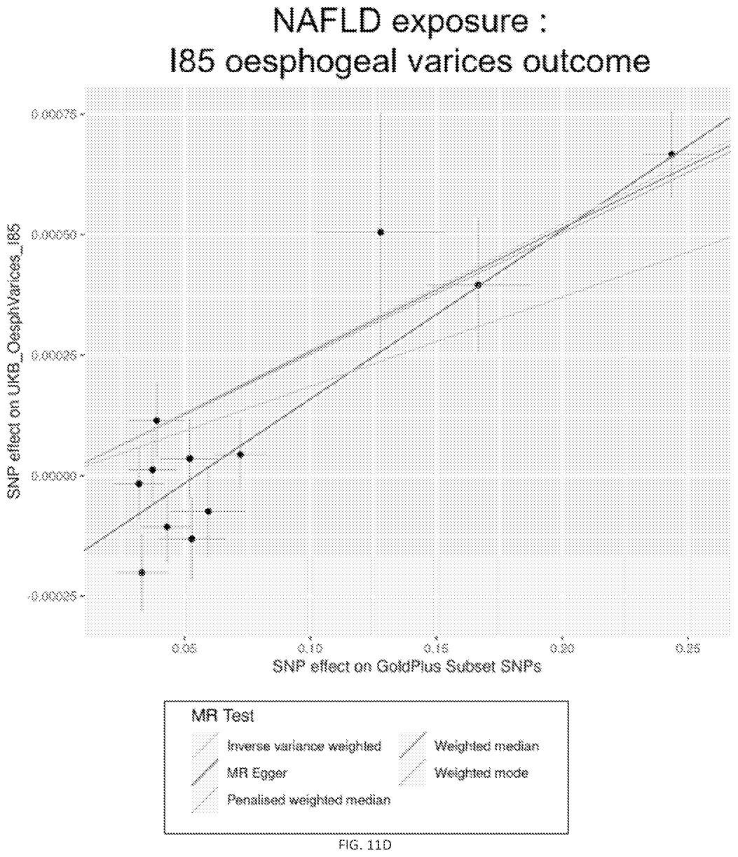

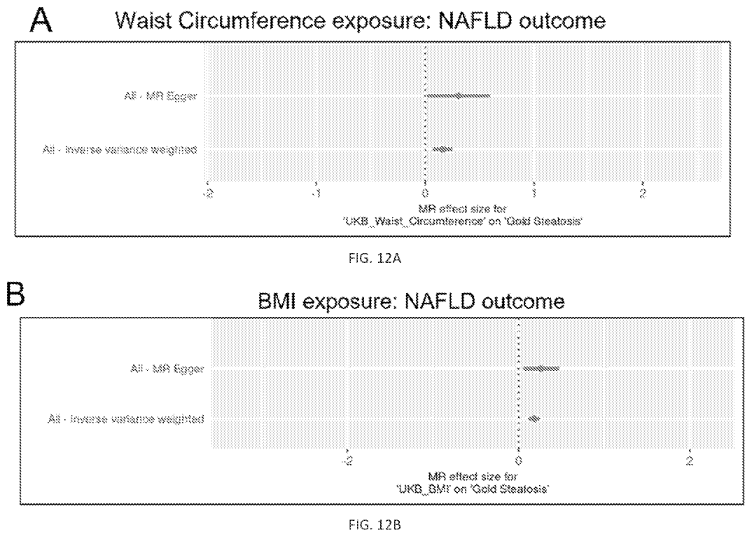

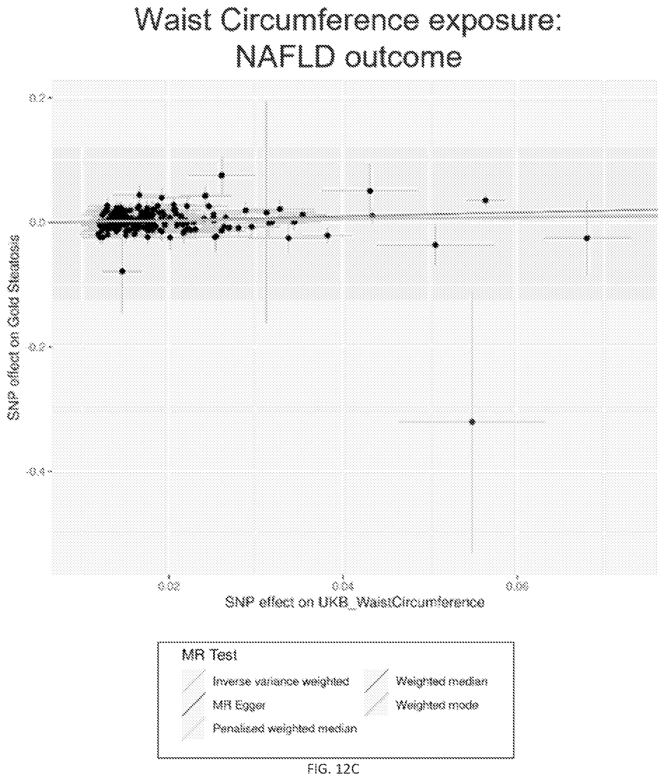

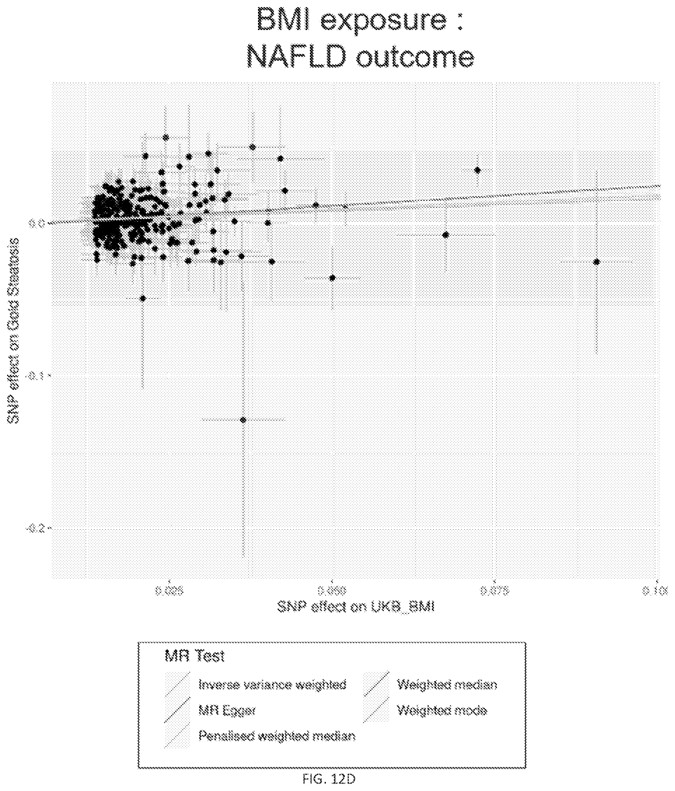

The causal effects of metabolic disorders, body composition measures and advanced liver disease were assessed on NAFLD. The GOLD Consortium was used as outcome and independent genome-wide significant variants (p-value<5E-08) from previously published GWAS (ebi.ac.uk/gwas/) as exposure. Increased BMI (OR=1.29, 95% CI=1.05-1.59, MR-Egger p-value=0.02) and waist circumference (OR=1.36, 95% CI=1.02-1.82, MR-Egger p-value=3.6E-02) increased risk of NAFLD (FIG. 12). The MR-Egger heterogeneity p-values were non-significant for BMI (p-value=0.051) and waist circumference (p-value=0.095). The MR-Egger pleiotropy p-values were non-significant for BMI (p-value=0.46) and waist circumference (p-value=0.296). The respective sensitivity analyses are shown in FIGS. 12C-12D.

### Example 6

### Effects on Liver Outcomes: NAFLD, Cirrhosis, Hepatocellular Carcinoma

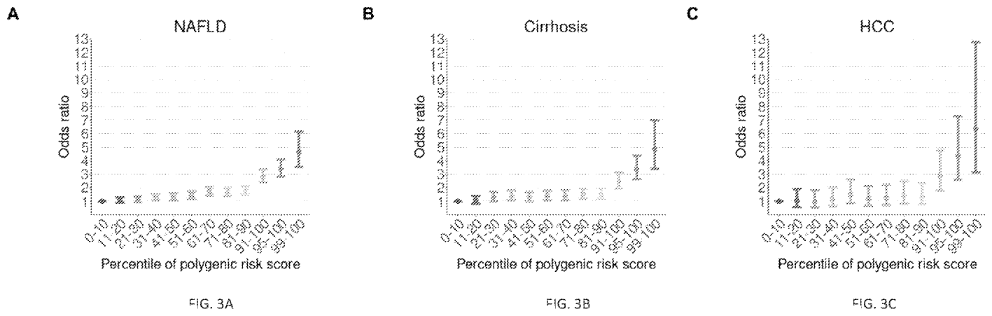

In order to assess the cumulative effects of NAFLD increasing variants on disease a PRS was constructed based on a weighted sum of dosage (multiethnic ancestry) of the NAFLD associated single variants (N=17) and its effect was assessed in an independent cohort: MGI (Table 3). Higher NAFLD PRS was strongly associated with an increased odds-ratio for NAFLD in MGI (FIG. 3A). Compared to those in the bottom decile of the PRS, individuals in the top 10%, 5%, and 1% had OR=2.83 (95% CI=2.39-3.34), 3.40 (95% CI=2.83-4.09), and 4.66 (95% CI=3.53-6.14) for NAFLD, respectively. Higher NAFLD PRS was also associated with increased odds of both MGI cirrhosis (top 10% OR 2.47 (95% CI=1.95-3.12), 5% 3.39 (95% CI=2.64-4.36), and 1% 4.87 (95% CI=3.39-7.00)) and MGI HCC (top 10% OR 2.91 (95% CI=1.77-4.78), 5% 4.35 (95% CI=2.59-7.31), and 1% 6.34 (95% CI=3.14-12.78)) (FIGS. 3B-3C).

### Example 7

### Pnpla3 and Diabetes in NAFLD Progression

NAFLD was defined based on ALT elevation and cirrhosis based on ICD codes in both cohorts. A Michigan Medicine cohort the ALT criterion has 88.6% specificity for NAFLD. In UKBB, the ALT definition of NAFLD was validated among the subset of participants who underwent liver magnetic resonance imaging with proton density fat fraction measurement and found that specificity of ALT elevations was 93.0% (3,272/3,515) for liver fat fraction >5.5%. ICD codes for cirrhosis demonstrated a positive predictive value of 86% in a Michigan Medicine cohort. A Michigan Medicine cohort was evaluated for sensitivity for ICD-10 codes for cirrhosis by evaluating patients with NAFLD (defined by ALT as above) who had imaging evidence of cirrhosis. It was found that 973/1251 (77.8%) of these patients had an ICD code for cirrhosis within 12 months of the date of the imaging study showing cirrhosis, implying that ICD codes have acceptable sensitivity for cirrhosis. ICD codes for cirrhosis were unable to be directly validated sensitivity in UK Biobank due to lack of access to a “gold standard” metric of cirrhosis.

The MGI cohort included 7,893 participants with NAFLD, among whom median age 52 years and approximately half were female. As expected in a NAFLD cobort, there was a high prevalence of diabetes (36%) and obesity (58%). Incident cirrhosis developed in 590 (6.8%) of MGI participants during a median follow-up of 72.5 months (IQR 45.9-100.5 months), yielding an incidence rate of 4.01 per 1,000 PY overall and 3.58 per 1,000 PY among those who did not have baseline advanced fibrosis (FIB4<2.67).

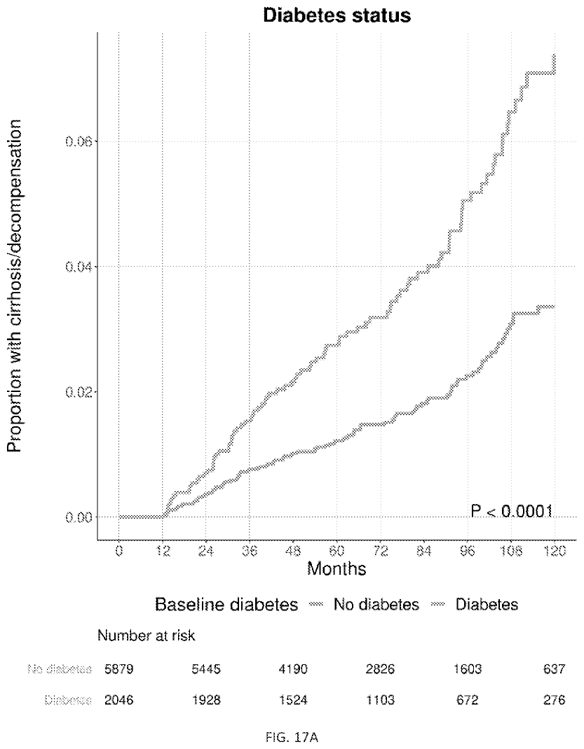

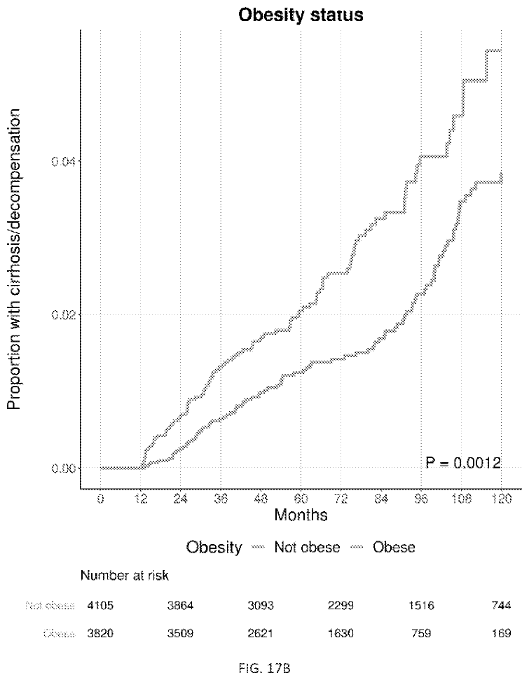

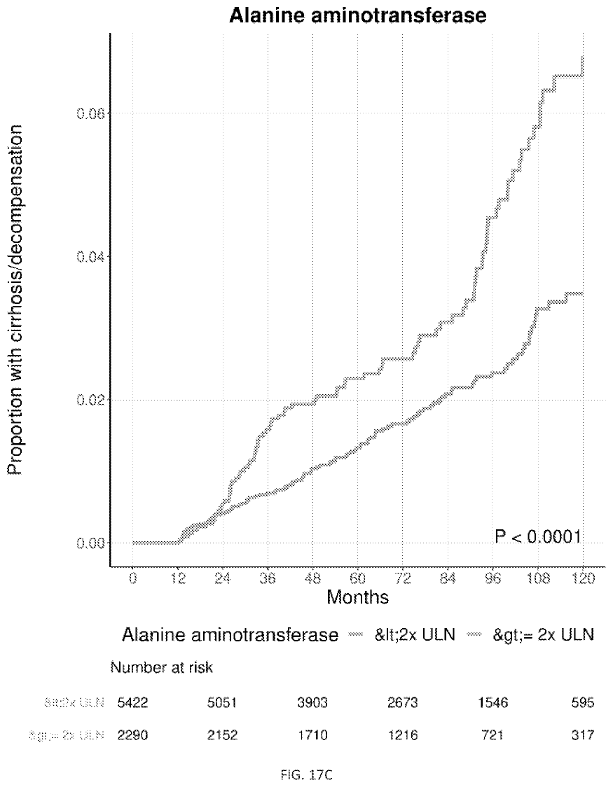

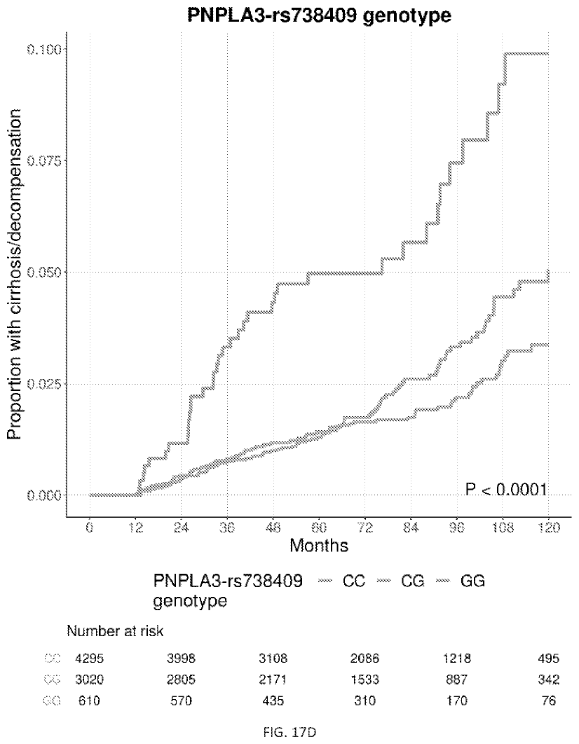

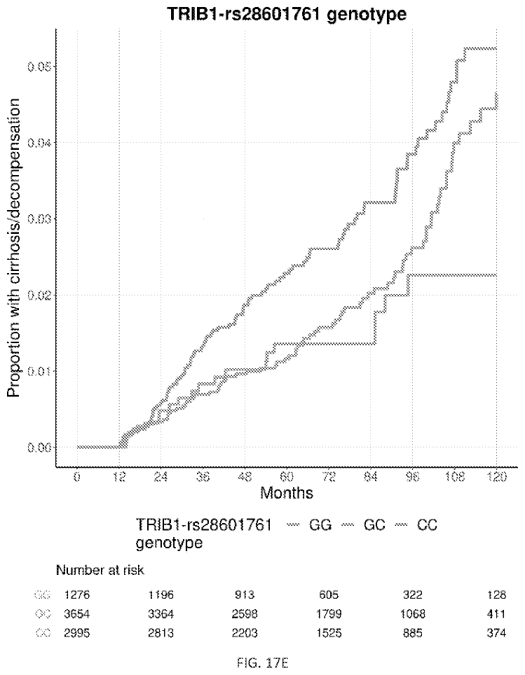

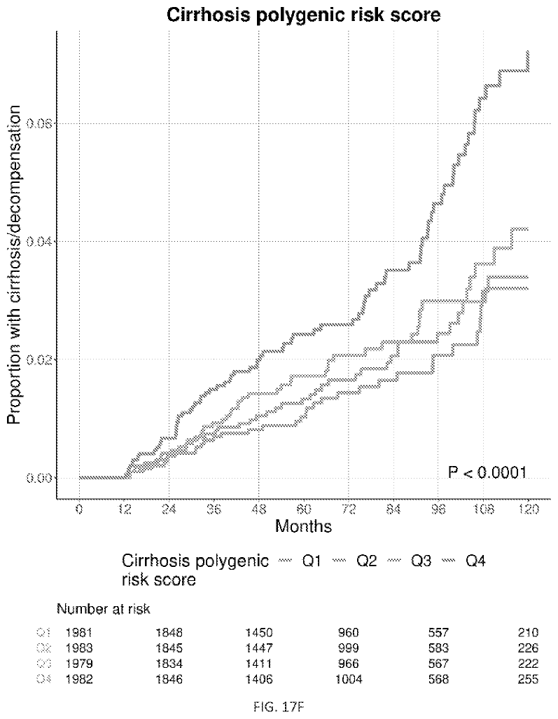

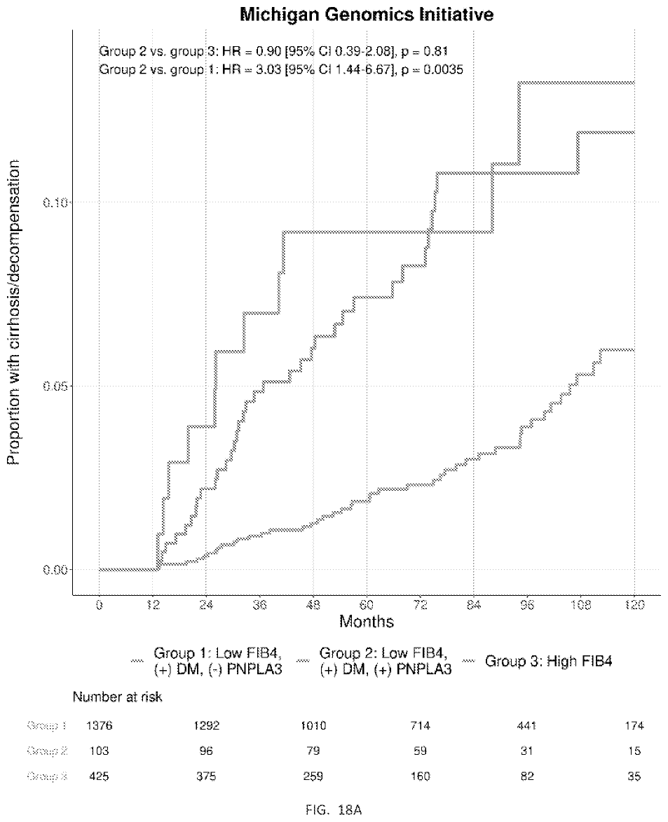

Univariate analysis showed that Fibrosis-4 (FIB4) score was strongly predictive of incident cirrhosis. Other risk factors included diabetes (hazard ratio [HR] 2.14 [95% confidence interval (CI) 1.60-2.85, p=3.0×10⁻⁷]), higher body mass index (HR 1.83 [95% CI 1.26-2.64, p=0.0014] for obese vs. lean/overweight), and elevated ALT (HR 2.00 [95% CI 1.48-2.69], p=5.2×10⁻⁶ for ≥ vs. <2x ULN) (FIG. 17). There was no significant association between hypertension or dyslipidemia and incident cirrhosis (p>0.05 for both comparisons).

Genetic variants previously associated with steatosis/cirrhosis were systematically evaluated. In MGI, only two of these individual variants were associated with increased rate of progression to cirrhosis: PNPLA3-rs738409-GG (vs.-CC) with HR 3.48 (95% CI 2.32-5.22, p=1.7×10⁻⁹) and TRIB1-rs28601761-CC (vs. GG) with HR 2.15 (95% CI 1.30-3.53, p=0.0026) (Table 4, FIG. 17). A previously-reported polygenic risk score for cirrhosis was associated with incident cirrhosis, but an effect was only observed at the highest risk quartile: HR 2.30 (1.53-3.46, p=6.3×10⁻⁵) vs. lowest quartile. Variants in TM6SF2, HSD17B13, and other previously reported risk loci were not significantly associated with incident cirrhosis. A sensitivity analysis including only patients without baseline advanced fibrosis (FIB4<2.67) yielded the same overall findings for the association with cirrhosis and genetic and non-genetic predictors as in the overall cohort.

A multivariable model for incident cirrhosis was generated including the most consistent predictors of incident cirrhosis in both cohorts, namely PNPLA3-rs738409-G, TRIB1-rs28601761-C, diabetes, obesity (categorized as obesity vs. lean/overweight), ALT level (categorized as ≥ vs. <2x ULN) and all remained significantly associated with incident cirrhosis with similar hazard ratios compared to the univariable analysis (Table 4). A sensitivity analysis in patients without baseline advanced fibrosis showed similar finding.

The remainder of the MGI analyses focused on patients without advanced fibrosis (FIB4<2.67) to determine whether genetic and/or environmental risk factors can identify a subgroup with more rapid disease progression.

PNPLA3 status was associated with increased risk of progression in the overall cohort of patients without advanced fibrosis (8.89 vs. 3.15 cases per 1,000 PY with PNPLA3-rs738409-GG vs.-CC/-CG genotype, respectively, p<0.0001) (Table 5). This association between PNPLA3 genotype and cirrhosis risk was even more notable when stratified by diabetes status, obesity status, and ALT level (Table 5). For example, among patients with diabetes, the cumulative incidence of cirrhosis was 3.2-fold higher in patients with PNPLA3-rs738409-GG vs.-CC/-CG genotype (16.4 vs. 5.1/1000 PY, respectively) (Table 5). A clinical risk score was generated based on diabetes, obesity, and ALT ≥2x ULN where each patient received 2 points if she had diabetes and 1 point each for obesity and ALT >2x ULN. Patients were divided in low, intermediate, and high risk (0-1, 2-3, and 4 points, respectively); these cutoffs were chosen because cumulative incidence of cirrhosis was similar in patients with 0 vs. 1, or 2 vs. 3 points. PNPLA3-rs738409-GG genotype was again associated with much higher cumulative incidence in the low-risk (6.3 vs. 2.3/1000 PY) and intermediate-risk groups (8.9 vs. 4.0/1000 PY; p<0.05 for both), with a trend toward higher cumulative incidence in the high-risk group as well (22.9 vs. 9.1/1000 PY, p=0.14) (Table 5). TRIB1-rs28601761-CC genotype was associated with higher risk of cirrhosis than-GG or-GC genotypes (4.6 vs. 3.0/1000 PY overall, p=0.0072). This association was also significant in patients without diabetes, with obesity, or with ALT ≥ 2x ULN. In models including gene-environment interaction terms (e.g., PNPLA3-rs738409-G dosage*diabetes status), the interaction terms were not significant for either PNPLA3 or TRIB1 genotype and any of the environmental predictors (p >0.05 for all).

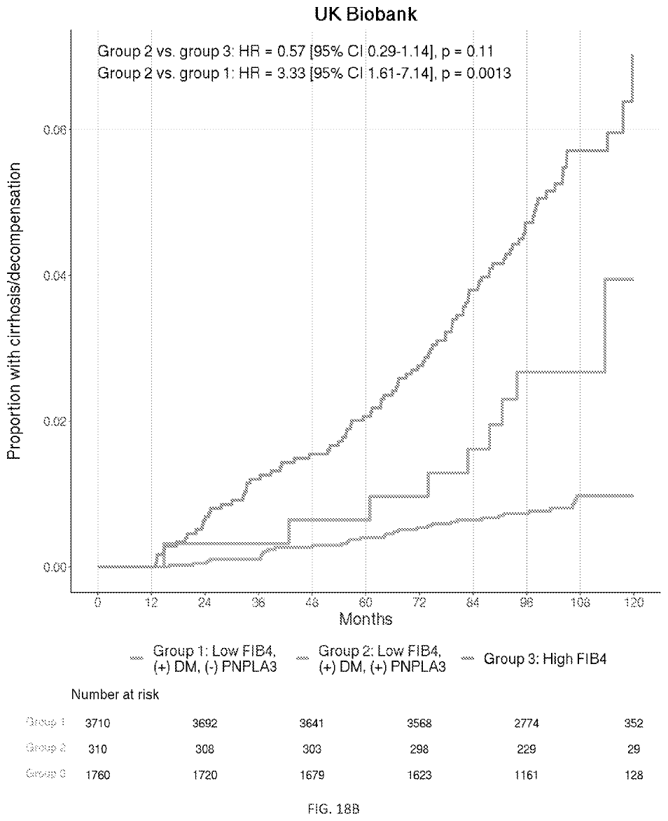

Patients with low baseline FIB4 scores, but with diabetes and PNPLA3-rs738409-GG, had an incidence of cirrhosis similar to that of patients with high baseline FIB4 (HR=0.90 [95% CI 0.39-2.08], p=0.81), and markedly higher than those with low FIB4 score, diabetes, and PNPLA3-rs738409-CC or-CG genotypes (HR 3.03 [95% CI 1.44-6.67], p=0.0035; both comparisons were after adjustment for age, sex, and principal components 1-10) (FIG. 18). Thus, persons with low FIB4 but PNPLA3-rs738409-GG genotype and diabetes had a rate of progression indistinguishable from those with high FIB4 scores.

The findings from MGI in patients with NAFLD in patients from an independent cohort were validated with UKBB. Unlike MGI, UKBB is a population-based cohort and as expected had a lower prevalence of comorbidities such as diabetes and obesity, and lower FIB4 scores. The UKBB cohort included 46,880 patients. In a median follow-up of 155.2 months (IQR 147.2-163.1 months), 191 (0.40%) developed incident cirrhosis, yielding an incident rate of 0.60 per 1000 PY overall and 0.39 per 1000 PY among those without baseline advanced fibrosis.

On univariable analysis, diabetes, obesity, elevated ALT, PNPLA3-rs738409-GG genotype were associated with incident cirrhosis, as was the case in MGI. The association between the TRIB1-rs28601761-G allele was not statistically significant. In UKBB unlike in MGI, TM6SF2-rs58542926-T associated with incident cirrhosis while the cirrhosis polygenic risk score did not. On multivariable analysis, the association between obesity and incident cirrhosis was no longer statistically significant (p=0.09) but there were otherwise no meaningful changes in the results. On sensitivity analysis including only those without advanced fibrosis at baseline, the overall findings were similar compared to the overall UKBB cohort.

Next, the combined effects on cirrhosis incidence of PNPLA3-rs738409 or TRIB1-rs28601761 genotype and the environmental factors of DM, obesity, or ALT elevations, were evaluated in UKBB in patients without baseline advanced fibrosis (e.g., FIB4<2.67). The associations between PNPLA3 genotype, metabolic risk factors, and incident cirrhosis were similar to the findings in MGI. PNPLA3-rs738409-GG genotype was associated with higher overall cumulative incidence of cirrhosis than-CC or-CG genotype (0.61 vs. 0.37/1000 PY, p=0.042) (Table 5). These differences were even greater among patients with diabetes or obesity: UKBB participants with diabetes or obesity and PNPLA3-rs738409-GG genotype had a >3-fold higher cumulative incidence of cirrhosis than did those with the-CC or-CG genotype (3.4 vs. 1.0 events/1000 PY for diabetes and 1.27 vs. 0.42 events/1000 PY for obesity; p<0.001 for both). Similarly, compared to PNPLA3-rs738409-CG or-CC genotype, the GG genotype was strongly associated with higher cumulative incidence of cirrhosis among patients with clinical risk score in the intermediate (1.31 vs. 0.53/1000 PY) or high range (5.78 vs. 1.78/1000 PY; p <0.05 for both). PNPLA3-rs738409 genotype was not significantly associated with incident cirrhosis among patients with low clinical risk score due to very small number of non-obese patients with incident cirrhosis and PNPLA3-rs738409-GG genotype (n=2) or among people without diabetes, obesity, or ALT≥2x ULN. TRIB1-rs28601761 genotype was not significantly associated with increased cumulative incidence of cirrhosis overall or in any subgroup in UKBB. As in MGI, gene-environment interaction terms were not significant between PNPLA3 or TRIB1 genotype and any of the above predictors (p>0.05 for all).

As with MGI, patients with low baseline FIB4 score and diabetes who carried the PNPLA3-rs738409-GG genotype had a cumulative incidence of cirrhosis similar to that of the patients with high baseline FIB4 (HR=0.57 [95% CI 0.29-1.14], p=0.11) and much greater than those with PNPLA3-rs738409-CC or-CG genotypes (HR=3.33 [95% CI 1.61-7.14], p=0.0013; both comparisons adjusted for age, sex, and principal components 1-10) (FIG. 18).

TABLE 1
Variants associated with NAFLD measures in GOLDPlus meta-analysis

| SNP ID | CHR:POS | EA | OA | EAF | Z-score | P-value | Gene Annotation |
|---|---|---|---|---|---|---|---|
| rs738408 | 22:44324730 | T | C | 0.22 | 35.21 | 1.53E−271 | PNPLA3 (D, E, L, N); SAMM50 (D) |
| rs58542926 | 19:19379549 | T | C | 0.07 | 22.76 | 1.19E−114 | TM6SF2 (D, B*, L, N); NCAN (D); SUGP1 |
| | | | | | | | (D); MAU2 (D) |
| rs429358 | 19:45411941 | T | C | 0.85 | 12.18 | 4.24E−34 | APOE (D, E*, L); APOC1 (D, L); TOMM40 |
| | | | | | | | (D); PVRL2 (D) |
| rs1260326 | 2:27730940 | T | C | 0.38 | 11.62 | 3.10E−31 | GCKR (D, E*, L, Q); SNX17 (D); C2orf16 (Q) |
| rs28601761 | 8:126500031 | C | G | 0.59 | 9.69 | 3.50E−22 | TRIB1 (D, L, N) |
| rs4918722 | 10:113947040 | C | T | 0.27 | 9.27 | 1.94E−20 | GPAM (D, E, L, N) |
| rs2807834 | 1:220970593 | G | T | 0.70 | 7.79 | 6.68E−15 | MARC1 (D, E*, L, N) |
| rs7661964 | 4:100505326 | A | T | 0.74 | 7.00 | 2.58E−12 | MTTP (D, E, L, N); C4orf17 (D) |
| rs7029757 | 9:132566666 | G | A | 0.91 | 6.68 | 2.38E−11 | TOR1B (N, Q); TOR1A (D) |
| rs1229984 | 4:100239319 | C | T | 0.95 | 6.56 | 5.57E−11 | ADH1B (D, E*, L, N); ADH4 (L); ADH1A (L) |
| rs17817449 | 16:53813367 | G | T | 0.39 | 6.15 | 7.56E−10 | FTO (N); RPGRIP1L (D) |
| rs79953491 | 2:165555539 | A | G | 0.88 | 5.95 | 2.71E−09 | COBLL1 (D, N, E); GRB14 (L) |
| rs112630404 | 19:7218635 | A | T | 0.18 | 5.85 | 4.88E−09 | INSR (D, N) |
| rs626283 | 19:54677001 | C | G | 0.43 | 5.75 | 8.99E−09 | TMC4 (E*, Q, N); MBOAT7 (Q); LENG1 (D) |
| rs4561528 | 17:17979099 | T | C | 0.35 | 5.57 | 2.52E−08 | SREBF1 (D, L); MYO15A (D, Q, E); DRG2 |
| | | | | | | | (D, N); DRC3 (D, Q, E); ATPAF2 (D, Q); |
| | | | | | | | TOM1L2 (D, Q); LLGL1 (Q); G1D4 (E); |
| rs10756038 | 9:10462423 | G | A | 0.72 | 5.47 | 4.58E−08 | PTPRD (D, N) |
| rs140201358 | 11:823586 | G | C | 0.01 | 5.50 | 3.81E−08 | PNPLA2 (D, E*, N) |
| rs738408 | 22:44324730 | T | C | 0.22 | 35.21 | 1.53E−271 | PNPLA3 (D, E, L, N); SAMM50 (D) |

CHR:POS, chromosome:position; EA, effect allele; OA, other allele; EAF, effect allele frequency.
Gene annotation tag: Gene prioritized by Depict analyses (D); Index variant is exonic (E*); Index variant is in strong LD (r2 > 0.85) with an exonic variant in the indicated gene (E); Index variant is within 1 MB of a variant in the indicated gene that is highly expressed in the liver using Gtex (L); Gene nearest to the index SNP (N); Index variant is in eQTL (FDR p < 0.05) with the indicated gene (Q).

TABLE 2
Independent GOLDPlus European meta-analysis NAFLD variants

| Gene | CHRBP | rsID | EA | OA | EAF | Zscore | P. value | HetPVal |
|---|---|---|---|---|---|---|---|---|
| PNPLA3 | 22:44324730 | rs738408 | t | c | 0.22 | 32.72 | 7.93E−235 | 3.49E−02 |
| TM6SF2 | 19:19388500 | rs8107974 | t | a | 0.08 | 22.86 | 1.19E−115 | 1.35E−07 |
| APOE | 19:45411941 | rs429358 | t | c | 0.85 | 12.37 | 3.61E−35 | 2.07E−02 |
| GCKR | 2:27598097 | rs4665972 | t | c | 0.40 | 10.68 | 1.25E−26 | 8.75E−02 |
| TRIB1 | 8:126506694 | rs112875651 | g | a | 0.60 | 9.34 | 9.32E−21 | 8.43E−03 |
| GPAM | 10:113949664 | rs10787429 | t | c | 0.28 | 8.66 | 4.87E−18 | 7.75E−01 |
| MARC1 | 1:220973563 | rs2642442 | t | c | 0.69 | 7.93 | 2.20E−15 | 1.17E−01 |
| MTTP | 4:100480915 | rs138764179 | t | c | 0.74 | 6.62 | 3.71E−11 | 9.74E−01 |
| ADH1B | 4:100239319 | rs1229984 | c | t | 0.97 | 6.54 | 6.22E−11 | 6.79E−01 |
| TOR1B | 9:132566666 | rs7029757 | g | a | 0.91 | 6.41 | 1.46E−10 | 5.44E−01 |
| TMC4/ | 19:54677001 | rs626283 | c | g | 0.43 | 6.15 | 7.76E−10 | 7.13E−01 |
| MBOAT7 | | | | | | | | |
| COBLL1/ | 2:165555539 | rs79953491 | a | g | 0.88 | 5.89 | 3.76E−09 | 2.19E−01 |
| GRB14 | | | | | | | | |
| SREBF1 | 17:17977355 | rs9303144 | c | t | 0.31 | 5.68 | 1.39E−08 | 8.64E−01 |
| INSR | 19:7202759 | rs8113542 | g | a | 0.26 | 5.53 | 3.19E−08 | 6.90E−02 |
| FTO | 16:53811788 | rs62033400 | g | a | 0.40 | 5.49 | 3.95E−08 | 8.27E−01 |
| PNPLA2 | 11:823586 | rs140201358 | g | c | 0.01 | 5.48 | 4.25E−08 | 4.69E−01 |
| TAMM41/ | 3:11916108 | rs559803897 | c | t | 0.99 | 5.47 | 4.50E−08 | 9.61E−01 |
| SYN2 | | | | | | | | |

TABLE 3
Independent GOLDPlus European meta-analysis NAFLD variants

| Multiethnic cohort | | |
|---|---|---|
| Covariates | N | Value in UKBB |
| mean age (SD) years | 43,293 | 64.2 (7.7) |
| % female | 43,293 | 51.5 |
| Diseases | N | Value in UKBB |
| mean PDFF (SD) | 43,293 | 3.9 (4.3) |
| European cohort | | |
| Covariates | N | Value in UKBB |
| mean age (SD) years | 41,834 | 64.3 (7.7) |
| % female | 41,834 | 51.7 |
| Diseases | N | Value in UKBB |
| mean PDFF (SD) | 41,834 | 3.9 (4.3) |

Each row gives number of UKBB participants for which a measurement is available/characteristic is known (N); and, the value, as either mean with standard deviation (SD), or N for cases and controls.

TABLE 4
Univariable and multivariable predictors of incident
cirrhosis in the Michigan Genomics Initiative cohort

| | Univariable | | Multivariable | |
|---|---|---|---|---|
| | Hazard ratio (95% | | Hazard ratio (95% | |
| Predictor | confidence interval) | P value | confidence interval) | P value |
| Diabetes | 2.14 (1.60-2.85) | 3.00E−07 | 2.01 (1.43-2.83) | 5.70E−05 |

Body mass index

| | | | | |
|---|---|---|---|---|
| Lean/overweight | (Referent) | | (Referent) | |
| Obese | 1.83 (1.26-2.64) | 0.0014 | 1.50 (1.04-2.18) | 0.031 |

Alanine aminotransferase

| | | | | |
|---|---|---|---|---|
| <2x ULN | (Referent) | | (Referent) | |
| >=2x ULN | 2.00 (1.48-2.69) | 5.2e−06 | 1.49 (1.06-2.10) | 0.024 |

PNPLA3-rs738409 genotype

| | | | | |
|---|---|---|---|---|
| CC | (Referent) | | (Referent) | |
| CG | 1.45 (1.06-1.98) | 0.02 | 1.43 (1.00-2.06) | 0.052 |
| GG | 3.48 (2.32-5.22) | 1.70E−09 | 3.24 (2.01-5.23) | 1.50E−06 |

TRIB1-rs28601761 genotype

| | | | | |
|---|---|---|---|---|
| GG | (Referent) | | (Referent) | |
| GC | 1.44 (0.87-2.38) | 0.15 | 1.20 (0.69-2.11) | 0.52 |
| CC | 2.15 (1.30-3.53) | 0.0026 | 1.91 (1.10-3.32) | 0.022 |

Models were run as Fine-Gray competing risk analyses. Results are shown as hazard ratio (95% confidence interval). In univariable models, effect of each specific predictor is shown after adjustment for age, sex, and genetic principal components 1-10 to account for ethnic variation. Multivariable results indicate hazard ratios for each predictor additionally adjusted for all of the other predictors shown in this table. ULN, upper limit of normal, defined as 19 U/L for women and 30 U/L for men.

TABLE 5
Cumulative incidence of cirrhosis stratified by PNPLA3
genotype, in patients without baseline advanced fibrosis,
in the Michigan Genomics Initiative and UK Biobank

| | PNPLA3-rs738409 genotype | | |
|---|---|---|---|
| Cohort | CC (lowest risk) or CG | GG (highest risk) | P value |

UK Biobank

| | | | |
|---|---|---|---|
| All | 0.37 (0.31-0.44) | 0.61 (0.36-0.97) | 0.042 |
| Diabetes | 1.00 (0.69-1.41) | 3.40 (1.55-6.45) | 0.00061 |
| Obesity | 0.42 (0.32-0.53) | 1.27 (0.74-2.03) | <0.0001 |
| ALT >= 2x ULN | 0.58 (0.46-0.73) | 0.82 (0.41-1.47) | 0.29 |
| Clinical risk score | | | |
| Low | 0.27 (0.21-0.34) | 0.15 (0.03-0.43) | 0.28 |
| Intermediate | 0.53 (0.38-0.72) | 1.31 (0.63-2.40) | 0.0091 |
| High | 1.78 (1.00-2.94) | 5.78 (1.88-13.50) | 0.017 |

Michigan Genomics Initiative

| | | | |
|---|---|---|---|
| All | 3.15 (2.59-3.80) | 8.89 (5.75-13.12) | <0.0001 |
| Diabetes | 5.09 (3.70-6.83) | 16.43 (8.20-29.40) | <0.0001 |
| Obesity | 3.70 (2.80-4.80) | 9.21 (4.76-16.09) | 0.0022 |
| ALT >= 2x ULN | 4.16 (3.00-5.62) | 13.85 (8.07-22.17) | <0.0001 |
| Clinical risk score | | | |
| Low | 2.27 (1.62-3.08) | 6.32 (2.73-12.45) | 0.0059 |
| Intermediate | 3.96 (2.74-5.53) | 8.89 (3.57-18.31) | 0.035 |
| High | 9.09 (4.54-16.26) | 22.89 (4.72-66.90) | 0.14 |

Cumulative incidence is shown as per 1,000 person-years (95% confidence interval), in the overall cohort and among patients with/without diabetes, obesity, or elevated alanine aminotransferase (ALT), and across the range of clinical risk score. Clinical risk score: low risk includes patients with no diabetes and no more than one of ALT >= 2x ULN or obesity; high risk includes those with diabetes, obesity, and ALT >= 2x ULN; and intermediate risk indicates all other patients. P value is for the association between PNPLA3 genotype (defined as rs738409-CC or -CG vs. -GG) and cumulative incidence of cirrhosis within each subgroup group. ULN, upper limit of normal, defined as 19 U/L for women or 30 U/L for men. Absence of baseline advanced fibrosis was defined as baseline Fibrosis-4 score <2.67.

TABLE 6
Top NAFLD SNPs

| Chromosome | Position | EA | OA | SNPID | Nearest Gene |
|---|---|---|---|---|---|
| 1 | 66554145 | T | C | rs11208797 | PDE4B |
| 1 | 110650174 | A | G | rs4839136 | LINC01397 |
| 1 | 172354992 | C | T | rs10752943 | DNM3 |
| 1 | 219448378 | C | T | rs12137855 | LYPLAL1 |
| 1 | 220970028 | G | A | rs2642438 | MTARC1 |
| 1 | 235327523 | G | A | rs112879517 | ARID4B |
| 2 | 21383514 | G | A | rs1712246 | TDRD15 |
| 2 | 25623603 | G | A | rs114018216 | DTNB |
| 2 | 27169393 | G | A | rs149219797 | DPYSL5 |
| 2 | 27730940 | T | C | rs1260326 | GCKR |
| 2 | 99738961 | A | G | rs6741772 | MRPL30 |
| 2 | 106914285 | C | T | rs34071542 | LOC402096 |
| 2 | 113841030 | A | G | rs6734238 | IL1RN |
| 2 | 137655519 | T | C | rs12999325 | THSD7B |
| 2 | 165528876 | C | T | rs13389219 | GRB14 |
| 3 | 5727851 | A | G | rs1840069 | MIR4790 |
| 3 | 12329783 | C | T | rs17036160 | PPARG |
| 3 | 50208406 | C | G | rs3774750 | SEMA3F |
| 4 | 17880416 | C | A | rs7700107 | LCORL |
| 4 | 77173739 | T | C | rs75132248 | FAM47E, FAM47E-STBD1 |
| 4 | 88230100 | T | G | rs10433937 | HSD17B13 |
| 4 | 92929643 | A | C | rs116160256 | LNCPRESS2 |
| 4 | 100239319 | C | T | rs1229984 | ADH1B |
| 4 | 100505326 | A | T | rs7661964 | MTTP |
| 4 | 103710930 | G | A | rs223454 | LOC102723704 |
| 5 | 22988560 | A | C | rs72750636 | CDH12 |
| 5 | 148342399 | T | C | rs2400785 | SH3TC2 |
| 6 | 25818755 | G | A | rs9461218 | SLC17A1 |
| 6 | 31587870 | T | A | rs2857694 | PRRC2A |
| 6 | 119484820 | C | G | rs601575 | MAN1A1 |
| 7 | 127383860 | T | A | rs1936811 | RSPO3 |
| 7 | 10521339 | T | C | rs58074807 | MGC4859 |
| 7 | 84532205 | C | G | rs782894 | SEMA3D |
| 7 | 98980659 | T | G | rs11973460 | ARPC1B |
| 8 | 6577140 | T | G | rs2911980 | AGPAT5 |
| 8 | 9183596 | A | G | rs4841132 | LOC157273 |
| 8 | 19824492 | T | C | rs13702 | LPL |
| 8 | 126482077 | A | G | rs2954021 | TRIB1, LINC00861 |
| 9 | 10462423 | G | A | rs10756038 | PTPRD |
| 9 | 15194625 | C | T | rs613981 | TTC39B |
| 9 | 16792621 | A | G | rs12553314 | BNC2 |
| 9 | 33109149 | A | C | rs13296330 | MIR12117 |
| 9 | 132566666 | G | A | rs7029757 | TOR1B |
| 10 | 36070931 | C | T | rs7073191 | PCAT5 |
| 10 | 78726447 | G | C | rs118028160 | KCNMA1-AS1 |
| 10 | 101912064 | T | C | rs2862954 | ERLIN1 |
| 10 | 113949664 | I | C | rs10787429 | GPAM, TECTB |
| 10 | 135378544 | T | C | rs9630002 | SYCE1 |
| 11 | 823586 | G | C | rs140201358 | PNPLA2 |
| 11 | 122013169 | C | T | rs531897 | MIR100HG |
| 12 | 19149829 | T | C | rs10505835 | PLEKHA5 |
| 12 | 21499248 | T | C | rs75208026 | SLCO1A2 |
| 12 | 82554772 | A | G | rs75159697 | LINC02426 |
| 12 | 85105077 | C | T | rs10862921 | SLC6A15 |
| 12 | 97557708 | G | T | rs7307068 | NEDD1 |
| 12 | 121424861 | A | G | rs7310409 | HNF1A |
| 12 | 124506631 | T | C | rs10773049 | ZNF664-RFLNA |
| 13 | 51106522 | T | A | rs1239948 | DLEU1 |
| 13 | 111019462 | A | C | rs4773169 | COL4A2 |
| 14 | 30067638 | A | G | rs7146602 | PRKD1 |
| 14 | 94844947 | T | C | rs28929474 | SERPINA1 |
| 15 | 73645403 | G | A | rs11630240 | HCN4 |
| 16 | 53806453 | G | A | rs56094641 | FTO |
| 16 | 68644795 | A | G | rs11643361 | CDH3 |
| 17 | 17979099 | T | C | rs4561528 | MYO15A |
| 17 | 64210580 | C | A | rs1801689 | APOH |
| 19 | 7218635 | A | T | rs112630404 | INSR |
| 19 | 18229208 | T | G | rs56252442 | MAST3 |
| 19 | 19379549 | T | C | rs58542926 | TM6SF2 |
| 19 | 33889593 | A | G | rs7256564 | PEPD |
| 19 | 45411941 | T | C | rs429358 | APOE |
| 19 | 54677001 | C | G | rs626283 | TMC4/MBOAT7 |
| 20 | 62336258 | T | C | rs6062497 | ARFRP1 |
| 22 | 17649774 | C | T | rs5748926 | IL17RA |
| 22 | 44324727 | G | C | rs738409 | PNPLA3 |

TABLE 7
CRISPRa and CRISPRi gRNAs

| Gene Target | gRNA SEQ ID NOs | | | | CRISPRa | CRISPRi | CRISPR-KO |
|---|---|---|---|---|---|---|---|
| CEBPA | 31-45 | 10177-10191 | 20358-20372 | ACSL3 | 1-15 | 10147-10161 | 20328-20342 |
| DGAT2 | 46-60 | 10192-10206 | 20373-20387 | SCAP | 16-30 | 10162-10176 | 20343-20357 |
| NUDT10 | 61-75 | 10207-10221 | 20388-20402 | FBXL14 | 661-675 | 10807-10821 | 20988-21002 |
| USP22 | 76-90 | 10222-10236 | 20403-20417 | CD27 | 676-690 | 10822-10836 | 21003-21017 |
| FAM47E | 91-105 | 10237-10251 | 20418-20432 | C5AR1 | 691-705 | 10837-10851 | 21018-21032 |
| HRC | 106-120 | 10252-10266 | 20433-20447 | INTS6 | 706-720 | 10852-10866 | 21033-21047 |
| PRADC1 | 121-135 | 10267-10281 | 20448-20462 | LYZL2 | 721-735 | 10867-10881 | 21048-21062 |
| IP6K1 | 136-150 | 10282-10296 | 20463-20477 | MAD2L1BP | 736-750 | 10882-10896 | 21063-21077 |
| DCAF8L1 | 151-165 | 10297-10311 | 20478-20492 | TAF2 | 751-765 | 10897-10911 | 21078-21092 |
| TTLL12 | 166-180 | 10312-10326 | 20493-20507 | SLC10A3 | 766-780 | 10912-10926 | 21093-21107 |
| PCGF1 | 181-195 | 10327-10341 | 20508-20522 | SEC31A | 781-768 | 10927-10941 | 21108-21122 |
| GAGE1 | 196-210 | 10342-10356 | 20523-20537 | NTPCR | 769-810 | 10942-10956 | 21123-21137 |
| PLEKHF2 | 211-225 | 10357-10371 | 20538-20552 | SCD | 811-825 | 10957-10971 | 21138-21152 |
| CHP1 | 226-240 | 10372-10386 | 20553-20567 | CCDC146 | 826-840 | 10972-10986 | 21153-21167 |
| HILPDA | 241-255 | 10387-10401 | 20568-20582 | PAX8 | 841-855 | 10987-11001 | 21168-21182 |
| GRIK5 | 256-270 | 10402-10416 | 20583-20597 | TMEM11 | 856-870 | 11002-11016 | 21183-21197 |
| PRR7 | 271-285 | 10417-10431 | 20598-20612 | SSTR5 | 871-885 | 11017-11031 | 21198-21212 |
| B3GNT6 | 286-300 | 10432-10446 | 20613-20627 | GRPR | 886-900 | 11032-11046 | 21213-21227 |
| PITPNA | 301-315 | 10447-10461 | 20628-20642 | GSN | 901-915 | 11047-11061 | 21228-21242 |
| JPH2 | 316-330 | 10462-10476 | 20643-20657 | ATXN2L | 916-930 | 11062-11076 | 21243-21257 |
| MAZ | 331-345 | 10477-10491 | 20658-20672 | HDAC4 | 931-945 | 11077-11091 | 21258-21272 |
| SLC4A2 | 346-360 | 10492-10506 | 20673-20687 | ZNF831 | 946-960 | 11092-11106 | 21273-21287 |
| CALHM2 | 361-375 | 10507-10521 | 20688-20702 | PREB | 961-975 | 11107-11121 | 21288-21302 |
| XAGE1A | 376-390 | 10522-10536 | 20703-20717 | OR6C75 | 976-990 | 11122-11134 | 21303-21317 |
| JUP | 391-405 | 10537-10551 | 20718-20732 | ACACA | 991-1005 | 11135-11149 | 21318-21332 |
| PRR5- | 406-420 | 10552-10566 | 20733-20747 | PSME3IP1 | 1006-1020 | 11150-11164 | 21333-21347 |
| ARHGAP8 | | | | ST8SIA5 | 1021-1035 | 11165-11179 | 21348-21362 |
| RTCB | 421-435 | 10567-10581 | 20748-20762 | GPAT4 | 1036-1050 | 11180-11194 | 21363-21377 |
| PHKG2 | 436-450 | 10582-10596 | 20763-20777 | HOXD9 | 1051-1065 | 11195-11209 | 21378-21392 |
| UPK1A | 451-465 | 10597-10611 | 20778-20792 | HNF4A | 1066-1080 | 11210-11224 | 21393-21407 |
| INPP5K | 466-480 | 10612-10626 | 20793-20807 | PCDHGA7 | 1081-1095 | 11225-11239 | 21408-21422 |
| GAMT | 481-495 | 10627-10641 | 20808-20822 | MIR6738 | 1096-1110 | 11240-11254 | 21423-21432 |
| MID1IP1 | 496-510 | 10642-10656 | 20823-20837 | OR2A5 | 1111-1125 | 11255-11269 | 21433-21447 |
| APOA4 | 511-525 | 10657-10671 | 20838-20852 | NPB | 1126-1140 | 11270-11284 | 21448-21462 |
| POU2AF3 | 526-540 | 10672-10686 | 20853-20867 | KRTAP1-5 | 1141-1154 | 11285-11299 | 21463-21477 |
| TMEM134 | 541-555 | 10687-10701 | 20868-20882 | KCNG2 | 1155-1169 | 11300-11314 | 21478-21492 |
| AIFM3 | 556-570 | 10702-10716 | 20883-20897 | ATIC | 1170-1184 | 11315-11329 | 21493-21507 |
| CD24 | 571-585 | 10717-10731 | 20898-20912 | MLLT1 | 1185-1199 | 11330-11344 | 21508-21322 |
| DHH | 586-600 | 10732-10746 | 20913-20927 | PRKAR1B | 1200-1214 | 11345-11359 | 21523-21537 |
| FEM1B | 601-615 | 10747-10761 | 20928-20942 | MIR6765 | 1215-1229 | 11360-11374 | 21538-21552 |
| SETDB1 | 616-630 | 10762-10776 | 20943-20957 | STK11 | 1230-1244 | 11375-11389 | 21553-21567 |
| FCER1G | 631-645 | 10777-10791 | 20958-20972 | JTB | 1245-1259 | 11390-11404 | 21568-21582 |
| KLK4 | 646-660 | 10792-10806 | 20973-20987 | ADCY9 | 1260-1274 | 11405-11419 | 21583-21597 |
| TAF7 | 1305-1319 | 11450-11464 | 21628-21642 | ZNF688 | 1275-1289 | 11420-11434 | 21598-21612 |
| CXXC1 | 1320-1334 | 11465-11479 | 21643-21657 | JAG1 | 1290-1304 | 11435-11449 | 21613-21627 |
| MIR6893 | 1335-1349 | 11480-11494 | 21658-21672 | MYOM2 | 1950-1964 | 12095-12109 | 22269-22283 |
| VEGFD | 1350-1364 | 11495-11509 | 21673-21687 | LRRC71 | 1965-1979 | 12110-12124 | 22284-22298 |
| SETD1A | 1365-1379 | 11510-11524 | 21688-21702 | BMPER | 1980-1994 | 12125-12139 | 22299-22313 |
| PMAIP1 | 1380-1394 | 11525-11539 | 21703-21717 | P4HTM | 1995-2009 | 12140-12154 | 22314-22328 |
| USP46 | 1395-1409 | 11540-11554 | 21718-21732 | TXNL1 | 2010-2024 | 12155-12169 | 22329-22343 |
| MIR1471 | 1410-1424 | 11555-11569 | 21733-21743 | B9D2 | 2025-2039 | 12170-12184 | 22344-22358 |
| FGFBP2 | 1425-1439 | 11570-11584 | 21744-21758 | AHRR | 2040-2054 | 12185-12199 | 22359-22373 |
| CHAD | 1440-1454 | 11585-11599 | 21759-21773 | OR6A2 | 2055-2069 | 12200-12214 | 22374-22388 |
| KCNC3 | 1455-1469 | 11600-11614 | 21774-21788 | HOXA13 | 2070-2084 | 12215-12229 | 22389-22403 |
| SCX | 1470-1484 | 11615-11629 | 21789-21803 | USP39 | 2085-2099 | 12230-12244 | 22404-22418 |
| SOX17 | 1485-1499 | 11630-11644 | 21804-21818 | FKBP1B | 2100-2114 | 12245-12259 | 22419-22433 |
| RALGAPA1 | 1500-1514 | 11645-11659 | 21819-21833 | SBSPON | 2115-2129 | 12260-12274 | 22434-22448 |
| NKX2-3 | 1515-1529 | 11660-11674 | 21834-21848 | RPIA | 2130-2144 | 12275-12289 | 22449-22463 |
| OR2C3 | 1530-1544 | 11675-11689 | 21849-21863 | PRDM13 | 2145-2159 | 12290-12304 | 22464-22478 |
| KMT2D | 1545-1559 | 11690-11704 | 21864-21878 | ENO2 | 2160-2174 | 12305-12319 | 22479-22493 |
| FRMD8 | 1560-1574 | 11705-11719 | 21879-21893 | ANGPT1 | 2175-2189 | 12320-12334 | 22494-22508 |
| IFNA8 | 1575-1589 | 11720-11734 | 21894-21908 | BNIP1 | 2190-2204 | 12335-12349 | 22509-22523 |
| CDYL2 | 1590-1604 | 11735-11749 | 21909-21923 | B3GNT4 | 2205-2219 | 12350-12364 | 22524-22538 |
| COL7A1 | 1605-1619 | 11750-11764 | 21924-21938 | HLA-F | 2220-2234 | 12365-12379 | 22539-22553 |
| CLDN1 | 1620-1634 | 11765-11779 | 21939-21953 | GSE1 | 2235-2249 | 12380-12394 | 22554-22568 |
| SSX2 | 1635-1649 | 11780-11794 | 21954-21968 | RASGEF1B | 2250-2264 | 12395-12409 | 22569-22583 |
| KLHL20 | 1650-1664 | 11795-11809 | 21969-21983 | PCSK1N | 2265-2279 | 12410-12424 | 22584-22598 |
| ATP13A1 | 1665-1679 | 11810-11824 | 21984-21998 | RAB11FIP1 | 2280-2294 | 12425-12439 | 22599-22613 |
| EGLN3 | 1680-1694 | 11825-11839 | 21999-22013 | POLDIP3 | 2295-2309 | 12440-12454 | 22614-22628 |
| CREBZF | 1695-1709 | 11840-11854 | 22014-22028 | MIR190A | 2310-2324 | 12455-12469 | 22629-22362 |
| RBM10 | 1710-1724 | 11855-11869 | 22029-22043 | TPSD1 | 2325-2339 | 12470-12484 | 22633-22647 |
| COMP | 1725-1739 | 11870-11884 | 22044-22058 | RHBDF1 | 2340-2354 | 12485-12499 | 22648-22662 |
| PTCHD4 | 1740-1754 | 11885-11899 | 22059-22073 | CHD7 | 2355-2369 | 12500-12514 | 22663-22677 |
| RIT2 | 1755-1769 | 11900-11915 | 22074-22088 | KLF9 | 2370-2381 | 12515-12529 | 22678-22692 |
| ALX4 | 1770-1784 | 11915-11929 | 22089-22103 | METTL22 | 2382-2396 | 12530-12544 | 22693-22707 |
| IL17D | 1785-1799 | 11930-11944 | 22104-22118 | AURKB | 2397-2411 | 12545-12559 | 22708-22722 |
| AMN1 | 1800-1814 | 11945-11959 | 22119-22133 | TSHZ1 | 2412-2426 | 12560-12574 | 22723-22737 |
| MIR378J | 1815-1829 | 11960-11974 | 22134-22148 | FLT3 | 2427-2441 | 12575-12589 | 22738-22752 |
| NF2 | 1830-1844 | 11975-11989 | 22149-22163 | HNF1A | 2442-2456 | 12590-12604 | 22753-22767 |
| INF2 | 1845-1859 | 11990-12004 | 22164-22178 | DISP2 | 2457-2471 | 12605-12619 | 22768-22782 |
| SLC26A10P | 1860-1874 | 12005-12019 | 22179-22193 | OTUD7B | 2472-2486 | 12620-12634 | 22783-22797 |
| FBXO5 | 1875-1889 | 12020-12034 | 22194-22208 | SLC7A4 | 2487-2501 | 12635-12649 | 22798-22812 |
| FBXO11 | 1890-1904 | 12035-12049 | 22209-22223 | POLR2F | 2502-2516 | 12650-12664 | 22813-22827 |
| ZNF395 | 1905-1919 | 12050-12064 | 22224-22238 | USF1 | 2517-2531 | 12665-12679 | 22828-22842 |
| EEF2K | 1920-1934 | 12065-12079 | 22239-22253 | LRP10 | 2532-2546 | 12680-12694 | 22843-22857 |
| NMRK2 | 1935-1949 | 12080-12094 | 22254-22268 | KLF1 | 2547-2561 | 12695-12709 | 22858-22872 |
| HAPSTR1 | 2592-2606 | 12740-12754 | 22903-22917 | REPIN1 | 2562-2576 | 12710-12724 | 22873-22887 |
| MIR6803 | 2607-2621 | 12755-12769 | 22918-22932 | VSTM2A | 2577-2591 | 12725-12739 | 22888-22902 |
| ELFN2 | 2622-2636 | 12770-12784 | 22933-22947 | FAM25C | 3236-3250 | 13385-13399 | 23548-23562 |
| MBTPS1 | 2637-2651 | 12785-12799 | 22948-22962 | COX6A2 | 3251-3265 | 13400-13414 | 23563-23577 |
| ALPK1 | 2652-2666 | 12800-12814 | 22963-22977 | HUWE1 | 3266-3280 | 13415-13429 | 23578-23592 |
| RBP5 | 2667-2681 | 12815-12829 | 22978-22992 | MIR6857 | 3281-3295 | 13430-13444 | 23593-23607 |
| CARD6 | 2682-2696 | 12830-12844 | 22993-23007 | CRHR2 | 3296-3310 | 13445-13459 | 23608-23622 |
| BRAT1 | 2697-2711 | 12845-12859 | 23008-23022 | UHRF1 | 3311-3325 | 13460-13474 | 23623-23637 |
| TRIM10 | 2712-2726 | 12860-12874 | 23023-23037 | SPSB4 | 3326-3340 | 13475-13489 | 23638-26352 |
| SH3BP5L | 2727-2741 | 12875-12889 | 23038-23052 | NOTCH1 | 3341-3355 | 13490-13504 | 23653-23667 |
| SUDS3 | 2742-2756 | 12890-12904 | 23053-23067 | NRL | 3356-3370 | 13505-13519 | 23668-23682 |
| THOC6 | 2757-2771 | 12905-12919 | 23068-20382 | SSTR1 | 3371-3385 | 13520-13534 | 23683-23697 |
| PCDHA12 | 2772-2785 | 12920-12934 | 23083-23097 | GTF3C1 | 3386-3400 | 13535-13549 | 23698-23712 |
| AREG | 2786-2800 | 12935-12949 | 23098-23112 | ITLN1 | 3401-3415 | 13550-13564 | 23713-23727 |
| GSC | 2801-2815 | 12950-12964 | 23113-23127 | KCNIP3 | 3416-3430 | 13565-13579 | 23728-23742 |
| TEX264 | 2816-2830 | 12965-12979 | 23128-23142 | ZSWIM8 | 3431-3445 | 13580-13594 | 23743-23757 |
| KDM4D | 2831-2845 | 12980-12994 | 23143-23157 | CPEB1 | 3446-3460 | 13595-13609 | 23758-23772 |
| OTUD7A | 2846-2860 | 12995-13009 | 23158-23172 | OR52B4 | 3461-3473 | 13610-13624 | 23773-23787 |
| ENTPD1 | 2861-2875 | 13010-13024 | 23173-23187 | KCNV1 | 3474-3488 | 13625-13639 | 23788-23802 |
| ARMC5 | 2876-2890 | 13025-13039 | 23188-23202 | SLC35C2 | 3489-3503 | 13640-13654 | 23803-23817 |
| IL27 | 2891-2905 | 13040-13054 | 23203-23217 | KRTAP19-7 | 3504-3516 | 13655-13669 | 23818-23832 |
| SLC16A9 | 2906-2920 | 13055-10369 | 23218-23232 | SERPINC1 | 3517-3531 | 13670-13684 | 23833-23847 |
| CYP7A1 | 2921-2935 | 13070-13084 | 23233-23247 | SLC4A8 | 3532-3546 | 13685-13699 | 23848-23862 |
| TBC1D10B | 2936-2950 | 13085-13099 | 23248-23262 | FMNL1 | 3547-3561 | 13700-13714 | 23863-23877 |
| TUBA3C | 2951-2965 | 13100-13114 | 23263-23277 | ZMYND19 | 3562-3576 | 13715-13729 | 23878-23892 |
| MED30 | 2966-2980 | 13115-13129 | 23278-23292 | PCNX3 | 3577-3591 | 13730-13744 | 23893-23907 |
| ALDH2 | 2981-2995 | 13130-13144 | 23293-23307 | RBM47 | 3592-3606 | 13745-13759 | 23908-23922 |
| CCR9 | 2996-3010 | 13145-13159 | 23308-23322 | AKR1C3 | 3607-3621 | 13760-13774 | 23923-23937 |
| MTDH | 3011-3025 | 13160-13174 | 23323-23337 | CD22 | 3622-3636 | 13775-13789 | 23938-23952 |
| CNN2 | 3026-3040 | 13175-13189 | 23338-23352 | ADRA2C | 3637-3651 | 13790-13804 | 23953-23967 |
| CEACAM4 | 3041-3055 | 13190-13204 | 23353-23367 | SERPINE1 | 3652-3666 | 13805-13819 | 23968-23982 |
| CLEC19A | 3056-3070 | 13205-13219 | 23368-23382 | POU3F2 | 3667-3681 | 13820-13834 | 23983-23997 |
| TRPS1 | 3071-3085 | 13220-13234 | 23383-23397 | CEACAM1 | 3682-3696 | 13835-13849 | 23998-24012 |
| ZNF784 | 3086-3100 | 13235-13249 | 23398-23412 | TCEA1 | 3697-3711 | 13850-13864 | 24013-24027 |
| NMUR1 | 3101-3115 | 13250-13264 | 23413-23427 | SPPL3 | 3712-3726 | 13865-13879 | 24028-24042 |
| MTFR1 | 3116-3130 | 13265-13279 | 23428-23442 | RAI14 | 3727-3741 | 13880-13894 | 24043-24057 |
| DOCK10 | 3131-3145 | 13280-13294 | 23443-23457 | NR2E1 | 3742-3756 | 13895-13909 | 24058-24072 |
| GPR135 | 3146-3160 | 13295-13309 | 23458-23472 | GLYR1 | 3757-3771 | 13910-13924 | 24073-24087 |
| MROH8 | 3161-3175 | 13310-13324 | 23473-23487 | B3GNTL1 | 3772-3786 | 13925-13939 | 24088-24102 |
| PLPPR3 | 3176-3190 | 13325-13339 | 23488-23502 | ZBTB20 | 3787-3801 | 13940-13954 | 24103-24117 |
| NRM | 3191-3205 | 13340-13354 | 23503-23517 | BICDL2 | 3802-3816 | 13955-13969 | 24118-24132 |
| TNIP2 | 3206-3220 | 13355-13369 | 23518-23532 | ITGB1 | 3817-3831 | 13970-13984 | 24133-24147 |
| WFDC10A | 3221-3235 | 13370-13384 | 23533-23547 | LTBP1 | 3832-3846 | 13985-13999 | 24148-24162 |
| HEATR9 | 3877-3891 | 14030-14044 | 24193-24207 | THBS4 | 3847-3861 | 14000-14014 | 24163-24177 |
| ZNE511 | 3892-3906 | 14045-14059 | 24208-24222 | TBC1D25 | 3862-3876 | 14015-14029 | 24178-24192 |
| MED16 | 3907-3921 | 14060-14074 | 24223-24237 | G6PC3 | 4520-4534 | 14675-14689 | 24838-24852 |
| PCDHGA9 | 3922-3935 | 14075-14089 | 24238-24252 | RBBP8NL | 4535-4549 | 14690-14704 | 24853-24867 |
| PRR15 | 3936-3950 | 14090-14104 | 24253-24267 | DTYMK | 4550-4564 | 14705-14719 | 24868-24882 |
| MIR6752 | 3951-3965 | 14105-14119 | 24268-24282 | HCLS1 | 4565-4579 | 14720-14734 | 24883-24897 |
| ZNF837 | 3966-3980 | 14120-14134 | 24283-24297 | MRPS26 | 4580-4594 | 14735-14749 | 24898-24912 |
| PARP4 | 3981-3995 | 14135-14149 | 24298-24312 | CYCS | 4595-4609 | 14750-14764 | 24913-24927 |
| HSPBP1 | 3996-4010 | 14150-14164 | 24313-24327 | BLCAP | 4610-4624 | 14765-14779 | 24928-24942 |
| TRIM56 | 4011-4025 | 14165-14179 | 24328-24342 | BRDT | 4625-4639 | 14780-14794 | 24943-24957 |
| LYZL1 | 4026-4040 | 14180-14194 | 24343-24357 | DDX60 | 4640-4654 | 14795-14809 | 24958-24972 |
| CREB3L2 | 4041-4055 | 14195-14209 | 24358-24372 | CNN1 | 4655-4669 | 14810-14824 | 24973-24987 |
| GJB6 | 4056-4070 | 14210-14224 | 24373-24387 | TNNC1 | 4670-4684 | 14825-14839 | 24988-25002 |
| FSCN2 | 4071-4085 | 14225-14239 | 24388-24402 | EQTN | 4685-4699 | 14840-14854 | 25003-25017 |
| PDIK1L | 4086-4100 | 14240-14254 | 24403-24417 | HPS6 | 4700-4714 | 14855-14869 | 25018-25032 |
| MIR7109 | 4101-4115 | 14255-14269 | 24418-24432 | RNASEH2A | 4715-4729 | 14870-14884 | 25033-25047 |
| ACKR2 | 4116-4129 | 14270-14284 | 24433-24447 | NRDC | 4730-4744 | 14885-14899 | 25048-25062 |
| TMIE | 4130-4144 | 14285-14299 | 24448-24462 | SSH1 | 4745-4759 | 14900-14914 | 25063-20577 |
| KIF1A | 4145-4159 | 14300-14314 | 24463-24477 | ADGRG4 | 4760 | — | 25078-25092 |
| IRF8 | 4160-4174 | 14315-14329 | 24478-24492 | CSMD2 | 4761-4775 | 14915-14928 | 25093-25107 |
| NLRP11 | 4175-4189 | 14330-14344 | 24493-24507 | ABHD5 | 4776-4790 | 14930-14944 | 25108-25122 |
| ATP8A1 | 4190-4204 | 14345-14359 | 24508-24522 | DNASE1L3 | 4791-4805 | 14945-14959 | 25123-25137 |
| DDT | 4205-4219 | 14360-14374 | 24523-24537 | PUM1 | 4806-4820 | 14960-14974 | 25138-25152 |
| CKMT2 | 4220-4234 | 14375-14389 | 24538-24552 | PPP2R2C | 4821-4835 | 14975-14989 | 25153-25167 |
| ACSM3 | 4235-4249 | 14390-14404 | 24553-24567 | VPS72 | 4836-4850 | 14990-15004 | 25168-25182 |
| STRAP | 4250-4264 | 14405-14419 | 24568-24582 | CGNL1 | 4851-4865 | 15005-15019 | 25183-25197 |
| MIR6850 | 4265-4279 | 14420-14434 | 24583-24597 | ACAD9 | 4866-4880 | 15020-15034 | 25198-25212 |
| CEBPE | 4280-4294 | 14435-14449 | 24598-24612 | ASNS | 4881-4895 | 15035-15049 | 25213-25227 |
| PRPF4B | 4295-4309 | 14450-14464 | 24613-24627 | NAT14 | 4896-4910 | 15050-15064 | 25228-25242 |
| GSDME | 4310-4324 | 14465-14479 | 24628-24642 | MRGBP | 4911-4925 | 15065-15079 | 25243-25257 |
| UBQLN3 | 4325-4339 | 14480-14494 | 24643-24657 | MRPS18A | 4926-4940 | 15080-15094 | 25258-25272 |
| IQCF2 | 4340-4354 | 14495-14509 | 24658-24672 | PRR20A | 4941-4955 | 15095-15109 | 25273-24287 |
| UBE2J2 | 4355-4369 | 14510-14524 | 24673-24687 | MYCBPAP | 4956-4970 | 15110-15124 | 25288-25302 |
| INSL3 | 4370-4384 | 14525-14539 | 24688-24702 | SAC3D1 | 4971-4985 | 15125-15139 | 25303-25317 |
| RILPL2 | 4385-4399 | 14540-14554 | 24703-24717 | SRSF10 | 4986-5000 | 15140-15154 | 25318-25332 |
| HDAC3 | 4400-4414 | 14555-14569 | 24718-24732 | MIR6878 | 5001-5013 | 15155-15169 | 25333-25339 |
| PMPCA | 4415-4429 | 14570-14584 | 24733-24747 | FLCN | 5014-5028 | 15170-15184 | 25340-25354 |
| RFC2 | 4430-4444 | 14585-14599 | 24748-24762 | MYBPHL | 5029-5043 | 15185-15199 | 25355-25369 |
| HID1 | 4445-4459 | 14600-14614 | 24763-24777 | ZNG1A | 5044-5058 | 15200-15214 | 25370-25384 |
| RETREG3 | 4460-4474 | 14615-14629 | 24778-24792 | OR5AR1 | 5059-5072 | 15215-15229 | 25385-25399 |
| GRSF1 | 4475-4489 | 14630-14644 | 24793-24807 | HUS1 | 5073-5087 | 15230-15244 | 25400-25414 |
| HADHB | 4490-4504 | 14645-14659 | 24808-24822 | COL6A1 | 5088-5102 | 15245-15259 | 25415-25429 |
| NDUFA6 | 4505-4519 | 14660-14674 | 24823-24837 | SASS6 | 5103-5117 | 15260-15274 | 25430-25444 |
| ELSPBP1 | 5148-5162 | 15305-15319 | 25474-25488 | MIR6129 | 5118-5132 | 15275-15289 | 25445-25458 |
| GCGR | 5163-5177 | 15320-15334 | 25489-25503 | PELO | 5133-5147 | 15290-15304 | 25459-25473 |
| RAB4B | 5178-5192 | 15335-15349 | 25504-25518 | ZZZ3 | 5808-5822 | 15965-15979 | 26132-26146 |
| SLC13A2 | 5193-5207 | 15350-15364 | 25519-25533 | SUMO4 | 5823-5837 | 15980-15994 | 26147-26161 |
| MIR6825 | 5208-5222 | 15365-15379 | 25534-25548 | HSF1 | 5838-5852 | 15995-16009 | 26162-26176 |
| NEK9 | 5223-5237 | 15380-15394 | 25549-25563 | SHOX2 | 5853-5867 | 16010-16024 | 26177-26191 |
| CYB5D1 | 5238-5252 | 15395-15409 | 25564-25578 | PSME3 | 5868-5882 | 16025-16039 | 26192-26206 |
| MAP1LC3B | 5253-5267 | 15410-15424 | 25579-25593 | TOR1A | 5883-5897 | 16040-16054 | 26207-26221 |
| ZNF829 | 5268-5282 | 15425-15439 | 25594-25608 | MKLN1 | 5898-5912 | 16055-16069 | 26222-26236 |
| INSIG1 | 5283-5297 | 15440-15454 | 25609-25623 | MROH2B | 5913-5927 | 16070-16084 | 26237-26251 |
| BLOC1S6 | 5298-5312 | 15455-15469 | 25624-25638 | MRPL18 | 5928-5942 | 16085-16099 | 26252-26266 |
| NAA38 | 5313-5327 | 15470-15484 | 25639-25653 | SP6 | 5943-5957 | 16100-16114 | 26267-26281 |
| TMX1 | 5328-5342 | 15485-15499 | 25654-25668 | FUCA1 | 5958-5972 | 16115-16129 | 26282-26296 |
| GIMAP8 | 5343-5257 | 15500-15514 | 25669-25683 | DNAAF10 | 5973-5987 | 16130-16144 | 26297-26311 |
| TARS2 | 5358-5372 | 15515-15529 | 25684-25698 | WDR44 | 5988-6002 | 16145-16159 | 26312-26326 |
| PTPRR | 5373-5287 | 15530-15544 | 25699-25713 | TBCD | 6003-6017 | 16160-16174 | 26327-26341 |
| ZNF654 | 5388-5402 | 15545-15559 | 25714-25728 | SAYSD1 | 6018-6032 | 16175-16189 | 26342-26356 |
| DNAH11 | 5403-5417 | 15560-15574 | 25729-25743 | ATG3 | 6033-6047 | 16190-16204 | 26357-26371 |
| CFAP36 | 5418-5432 | 15575-15589 | 25744-25758 | CC2D1B | 6048-6062 | 16205-16219 | 26372-26386 |
| EIF4B | 5433-5447 | 15590-15604 | 25759-25773 | ZMPSTE24 | 6063-6077 | 16220-16234 | 26387-26401 |
| EMC9 | 5448-5462 | 15605-15619 | 25774-25788 | KLK9 | 6078-6092 | 16235-16249 | 26402-26416 |
| HIGD1A | 5463-5477 | 15620-15634 | 25789-25803 | NBPF4 | 6093-6107 | 16250-16264 | 26417-26431 |
| KMT2B | 5478-5492 | 15635-15649 | 25804-25818 | CCZ1B | 6108-6122 | 16265-16279 | 26432-26446 |
| SPTBN5 | 5493-5507 | 15650-15664 | 25819-25833 | ODAD4 | 6123-6137 | 16280-16294 | 26447-26461 |
| SCYL1 | 5508-5522 | 15665-15679 | 25834-25848 | SYT13 | 6138-6152 | 16295-16309 | 26462-26476 |
| TMEM199 | 5523-5537 | 15680-15694 | 25849-25863 | ZFR | 6153-6167 | 16310-16324 | 26477-26491 |
| PNPT1 | 5538-5552 | 15695-15709 | 25864-25878 | STK40 | 6168-6182 | 16325-16339 | 26492-26506 |
| RBBP4 | 5553-5567 | 15710-15724 | 25879-25893 | RASGEF1C | 6183-6197 | 16340-16354 | 26507-26521 |
| TBX21 | 5568-5582 | 15725-15739 | 25894-25908 | NPRL2 | 6198-6212 | 16355-16369 | 26522-26536 |
| ZRSR2 | 5583-5597 | 15740-15754 | 25909-25923 | CTAGE4 | 6213-6227 | 16370-16384 | 26537-26551 |
| LHX9 | 5598-5612 | 15755-15769 | 25924-25938 | NAA10 | 6228-6242 | 16385-16399 | 26552-26566 |
| HPCA | 5613-5642 | 15770-15799 | 25939-25968 | CSTF2 | 6243-6257 | 16400-16414 | 26567-26581 |
| ORC5 | 5643-5657 | 15800-15814 | 25969-25983 | NDUFAF3 | 6258-6272 | 16415-16429 | 26582-26596 |
| CCDC172 | 5658-5672 | 15815-15829 | 25984-25998 | RASL10B | 6273-6287 | 16430-16444 | 26597-26611 |
| CDC14A | 5673-5687 | 15830-15844 | 25999-26013 | UNC13C | 6288-6302 | 16445-16459 | 26612-26626 |
| ANGPTL6 | 5688-5702 | 15845-15859 | 26014-26028 | WASHC1 | 6303-6317 | 16460-16474 | 26627-26641 |
| RFC5 | 5703-5717 | 15860-15874 | 26029-26043 | C16orf87 | 6318-6332 | 16475-16489 | 26642-26656 |
| NSUN2 | 5718-5732 | 15875-15889 | 26044-26058 | TVP23B | 6333-6347 | 16490-16504 | 26657-26671 |
| SLC25A12 | 5733-5747 | 15890-15904 | 26059-26073 | TM4SF5 | 6348-6362 | 16505-16519 | 26672-26686 |
| MIR6760 | 5748-5762 | 15905-15919 | 26074-26086 | LSM11 | 6363-6377 | 16520-16534 | 26687-26701 |
| RPS28 | 5763-5777 | 15920-15934 | 26087-26101 | ATP11A | 6378-6392 | 16535-16549 | 26702-26716 |
| TMEM9B | 5778-5792 | 15935-15949 | 26102-26116 | CIDEB | 6393-6407 | 16550-16564 | 26717-26731 |
| NAA25 | 5793-5807 | 15950-15964 | 26117-26131 | VPS18 | 6408-6422 | 16565-16579 | 26732-26746 |
| H2AC16 | 6453-6467 | 16610-16624 | 26777-26791 | FAM120A | 6423-6437 | 16580-16594 | 26747-26761 |
| NEDD8 | 6468-6482 | 16625-16639 | 26792-26806 | PIGN | 6438-6452 | 16595-16609 | 26762-26776 |
| CFLAR | 6483-6492 | 16640-16654 | 26807-26821 | SYDE2 | 7090-7104 | 17254-17268 | 27422-27436 |
| LRRC2 | 6493-6507 | 16655-16669 | 26822-26836 | ASCL3 | 7105-7119 | 17269-17283 | 27437-27451 |
| CCND1 | 6508-6522 | 16670-16684 | 26837-26851 | SPATA21 | 7120-7134 | 17284-17298 | 27452-27466 |
| MTMR2 | 6523-6537 | 16685-16699 | 26852-26866 | PNPLA2 | 7135-7149 | 17299-17313 | 27467-27481 |
| CTPS1 | 6538-6552 | 16700-16714 | 26867-26881 | SULT1A4 | 7150-7164 | 17314-17328 | 27482-27496 |
| RPLPO | 6553-6567 | 16715-16729 | 26882-26896 | FOXF1 | 7165-7179 | 17329-17343 | 27497-27511 |
| NKAIN4 | 6568-6582 | 16730-16744 | 26897-26911 | ADSS2 | 7180-7194 | 17344-17358 | 27512-27526 |
| NOL10 | 6583-6597 | 16745-16759 | 26912-26926 | ALYREF | 7195-7209 | 17359-17373 | 27527-27541 |
| MT1G | 6598-6612 | 16760-16774 | 26927-26941 | FDFT1 | 7210-7224 | 17374-17388 | 27542-27556 |
| DUSP7 | 6613-6627 | 16775-16789 | 26942-26956 | GABRB3 | 7225-7239 | 17389-17403 | 27557-27571 |
| TRIR | 6628-6642 | 16790-16804 | 26957-26971 | MRGPRX3 | 7240-7254 | 17404-17418 | 27572-27586 |
| HINT1 | 6643-6657 | 16805-16819 | 26972-26986 | UNC45A | 7255-7269 | 17419-17433 | 27587-27601 |
| AGMO | 6658-6672 | 16820-16834 | 26987-27001 | HABP4 | 7270-7284 | 17434-17448 | 27602-27616 |
| DAGLA | 6673-6687 | 16835-16849 | 27002-27016 | IRAG1 | 7285-7299 | 17449-17463 | 27617-27631 |
| LRRC39 | 6688-6699 | 16850-16864 | 27017-27031 | USP10 | 7300-7314 | 17464-17478 | 27632-27646 |
| TRIM47 | 6700-6914 | 16865-16879 | 27032-27046 | SPACA9 | 7315-7329 | 17479-17493 | 27647-27662 |
| CATSPER3 | 6715-6729 | 16880-16894 | 27047-27061 | VCAM1 | 7330-7344 | 17494-17508 | 27662-27676 |
| CD151 | 6730-6744 | 16895-16909 | 27062-27076 | ECM2 | 7345-7359 | 17509-17519 | 27677-27691 |
| PSD4 | 6745-6759 | 16910-16924 | 27077-27091 | GINS3 | 7360-7374 | 17520-17534 | 27692-27706 |
| RNF17 | 6760-6774 | 16925-16939 | 27092-27106 | ILK | 7375-7389 | 17535-17549 | 27707-27721 |
| IST1 | 6775-6789 | 16940-16954 | 27107-27121 | COG4 | 7390-7404 | 17550-17564 | 27722-27736 |
| TMPPE | 6790-6804 | 16955-16969 | 27122-27136 | KLHL1 | 7405-7419 | 17565-17579 | 27737-27751 |
| FBXL3 | 6805-6819 | 16970-16984 | 27137-27151 | HECW1 | 7420-7434 | 17580-17594 | 27752-27766 |
| CD3G | 6820-6834 | 16985-16999 | 27152-27166 | GPR171 | 7435-7443 | 17595-17609 | 27767-27781 |
| ZNF420 | 6835-6849 | 17000-17014 | 27167-27181 | MTRNR2L1 | 7444-7458 | 17610-17624 | 30530-30531 |
| LHFPL1 | 6850-6864 | 17015-17029 | 27182-27196 | IFNW1 | 7459-7473 | 17625-17639 | 27782-27796 |
| SOX9 | 6865-6879 | 17030-17044 | 27197-27211 | MIR590 | 7474-7488 | 17640-17654 | 27797-27799 |
| RSRC2 | 6880-6894 | 17045-17059 | 27212-27226 | SSU72 | 7489-7503 | 17655-17669 | 27800-27814 |
| CAMK1 | 6895-6909 | 17060-17074 | 27227-27241 | MST1L | 7504-7518 | 17670-17684 | 27815-27829 |
| C2CD2L | 6910-6924 | 17075-17089 | 27242-27256 | TNFRSF13C | 7519-7533 | 17685-17699 | 27830-27844 |
| PHF2 | 6925-6939 | 17090-17104 | 27257-27271 | MIR1243 | 7534-7546 | 17700-17714 | 27845-27851 |
| CPSF3 | 6940-6954 | 17105-17119 | 27272-27286 | SYNCRIP | 7547-7561 | 17715-17729 | 27852-27866 |
| MYH4 | 6955-6969 | 17120-17133 | 27287-27301 | OR4C46 | 7562-7573 | 17730-17744 | 27867-27881 |
| KLHDC4 | 6970-6984 | 17134-17148 | 27302-27316 | NLRP13 | 7574-7583 | 17745-17759 | 27882-27896 |
| DXO | 6985-6999 | 17149-17163 | 27317-27331 | SEC62 | 7584-7598 | 17760-17774 | 27897-27911 |
| FCHO2 | 7000-7014 | 17164-17178 | 27332-27346 | H4C11 | 7599-7613 | 17775-17789 | 27912-27926 |
| RHOA | 7015-7029 | 17179-17193 | 27347-27361 | HTR3A | 7614-7628 | 17790-17804 | 27927-27941 |
| MIR1199 | 7030-7044 | 17194-17208 | 27362-27376 | PAFAH1B2 | 7629-7643 | 17805-17819 | 27942-27956 |
| FBXO10 | 7045-7059 | 17209-17223 | 27377-27391 | DTNA | 7644-7658 | 17820-17834 | 27957-27971 |
| PROCA1 | 7060-7074 | 17224-17238 | 27392-27406 | CTNNBL1 | 7659-7673 | 17835-17849 | 27972-27986 |
| IGSF5 | 7075-7089 | 17239-17253 | 27407-27421 | TGIF1 | 7674-7688 | 17850-17864 | 27987-28001 |
| ZMYND8 | 7719-7733 | 17895-17909 | 28032-28046 | RPN1 | 7689-7703 | 17865-17879 | 28002-28016 |
| MEF2B | 7734-7748 | 17910-17924 | 28047-28061 | RBP2 | 7704-7718 | 17880-17894 | 28017-28031 |
| CYBSD2 | 7749-7763 | 17925-17939 | 28062-28076 | NAALADL1 | 8337-8351 | 18531-18545 | 28669-28683 |
| GPR141 | 7764-7778 | 17940-17954 | 28077-28091 | IFT43 | 8352-8366 | 18546-18560 | 28684-28698 |
| RCN3 | 7779-7793 | 17955-17969 | 28092-28106 | EMC6 | 8367-8381 | 18561-18575 | 28699-28713 |
| TCF19 | 7794-7808 | 17970-17984 | 28107-28121 | ZACN | 8382-8396 | 18576-18590 | 28714-28728 |
| TMEM217 | 7809-7823 | 17985-17999 | 28122-28136 | DHX34 | 8397-8411 | 18591-18605 | 28729-28743 |
| RAD9A | 7824-7838 | 18000-18014 | 28137-28151 | TARP | 8412-8426 | 18606-18620 | 28744-28758 |
| KANSL1 | 7839-7853 | 18015-18029 | 28152-28166 | FRAT2 | 8427-8441 | 18621-18635 | 28759-28773 |
| OR4F16 | 7854-7868 | 18030-18044 | 28167-28181 | FIBIN | 8442-8456 | 18636-18650 | 28774-28788 |
| DHFR | 7869-7883 | 18045-18059 | 28182-28196 | DLX5 | 8457-8471 | 18651-18665 | 28789-28803 |
| ZNF510 | 7884-7898 | 18060-18074 | 28197-28211 | TRMT112 | 8472-8486 | 18666-18680 | 28804-28818 |
| TMEM14EP | 7899-7901 | 18075-18089 | 28212-28226 | MRPS6 | 8487-8501 | 18681-18695 | 28819-28833 |
| TICAM1 | 7902-7916 | 18090-18104 | 28227-28241 | GPR85 | 8502-8516 | 18696-18710 | 28834-28848 |
| CACNB2 | 7917-7931 | 18105-18119 | 28242-28256 | GRAMD4 | 8517-8531 | 18711-18725 | 28849-28863 |
| TMEM233 | 7932-7946 | 18120-18134 | 28257-28271 | PSMD9 | 8532-8546 | 18726-18740 | 28864-28878 |
| PRELID3B | 7947-7961 | 18135-18149 | 28272-28286 | NUDT8 | 8547-8561 | 18741-18755 | 28879-28893 |
| DIDO1 | 7962-7976 | 18150-18164 | 28287-28301 | POTEJ | 8562-8576 | 18756-18770 | 28894-28908 |
| SPG21 | 7977-7991 | 18165-18179 | 28302-28316 | ADAM19 | 8577-8591 | 18771-18785 | 28909-28923 |
| MIR6721 | 7992-8006 | 18180-18194 | 28317-28331 | SLC9A8 | 8592-8606 | 18786-18800 | 28924-28938 |
| MAJIN | 8007-8021 | 18195-18209 | 28332-28346 | RPL9 | 8607-8621 | 18801-18815 | 28939-28953 |
| GRM5 | 8022-8036 | 18210-18224 | 28347-28361 | GUCA2B | 8622-8636 | 18816-18830 | 28954-28968 |
| OR5A2 | 8037-8051 | 18225-18239 | 28362-28376 | PDE4B | 8637-8651 | 18831-18845 | 28969-28983 |
| SEMA6B | 8052-8066 | 18240-18254 | 28377-28391 | LINC01397 | 8652-8666 | 18846-18860 | 28984-28998 |
| FHDC1 | 8067-8081 | 18255-18269 | 28392-28406 | LINC00626 | 8667-8681 | 18861-18875 | 28999-29013 |
| SLC6A20 | 8082-8096 | 18270-18284 | 28407-28421 | DNM3 | 8682-8697 | 18876-18890 | 29014-29028 |
| FAM169A | 8097-8111 | 18285-18299 | 28422-28436 | ZBTB41 | 8697-8712 | 18891-18905 | 29029-29043 |
| CFAP77 | 8112-8126 | 18300-18314 | 28437-28451 | MTARC1 | 8712-8726 | 18906-18920 | 29044-20958 |
| ARF1 | 8127-8141 | 18315-18329 | 28452-28466 | ARID4B | 8727-8741 | 18921-18935 | 29059-20973 |
| HTN1 | 8142-8156 | 18330-18335 | 28467-28473 | TDRD15 | 8742-8756 | 18936-18950 | 29074-29088 |
| MIR6785 | 8157-8171 | 18336-18350 | 28474-28488 | DTNB | 8757-8771 | 18951-18965 | 29089-29103 |
| TESMIN | 8172-8186 | 18351-18365 | 28489-28503 | DPYSL5 | 8772-8786 | 18966-18980 | 29104-29118 |
| SCNN1D | — | 18366-18380 | 28504-28518 | GCKR | 8787-8801 | 18981-18995 | 29119-29133 |
| C11orf86 | 8187-8201 | 18381-18395 | 28519-28533 | MRPL30 | 8802-8816 | 18996-19010 | 29134-29148 |
| DDI2 | 8202-8216 | 18396-18410 | 28534-28548 | THSD7B | 8817-8831 | 19011-19025 | 29149-29163 |
| ZNF568 | 8217-8231 | 18411-18425 | 28549-28563 | COBLL1 | 8832-8846 | 19026-19040 | 29164-29178 |
| ADGRE3 | 8232-8246 | 18426-18440 | 28564-28578 | MIR4790 | 8847-8861 | 19041-19047 | 29179-29180 |
| PRPF38B | 8247-8261 | 18441-18455 | 28579-28593 | SEMA3F | 8862-8876 | 19048-19062 | 29181-29195 |
| SFMBT1 | 8262-8276 | 18456-18470 | 28594-28608 | CPZ | 8877-8891 | 19063-19077 | 29196-29210 |
| CAPZB | 8277-8291 | 18471-18485 | 28609-28623 | LINC02494 | 8892-8901 | 19078-19092 | 29211-29225 |
| LIN28B | 8292-8306 | 18486-18500 | 28624-28638 | LNCPRESS2 | 8902-8916 | 19093-19107 | 29226-29240 |
| CNEP1R1 | 8307-8321 | 18501-18515 | 28639-28653 | UNC5C | 8917-8931 | 19108-19122 | 29241-29255 |
| LDAH | 8322-8336 | 18516-18530 | 28654-28668 | ADH1B | 8932-8946 | 19123-19133 | 29256-29270 |
| CDH12 | 8977-8991 | 19164-19178 | 29301-29315 | MTTP | 8947-8961 | 19134-19148 | 29271-29285 |
| SH3TC2 | 8992-9006 | 19179-19193 | 29316-29330 | TRIM2 | 8962-8976 | 19149-19163 | 29286-29300 |
| SLC17A1 | 9007-9021 | 19194-19208 | 29331-29345 | C17orf113 | 9607-9621 | 19788-19802 | 29922-29936 |
| HCG15 | 9022-9036 | 19209-19223 | 29346-29360 | APOH | 9622-9636 | 19803-19817 | 29937-29951 |
| TRIM26 | 9037-9051 | 19224-19238 | 29361-29375 | INSR | 9637-9651 | 19818-19832 | 29952-29966 |
| PRRC2A | 9052-9066 | 19239-19253 | 29376-29390 | JUND | 9652-9666 | 19833-19847 | 29967-29981 |
| HLA-DOA | 9067-9081 | 19254-19268 | 29391-29405 | TM6SF2 | 9667-9681 | 19848-19862 | 29982-29996 |
| MAN1A1 | 9082-9096 | 19269-19283 | 29406-29420 | APOE | 9682-9696 | 19863-19877 | 29997-30011 |
| RSPO3 | 9097-9111 | 19284-19298 | 29421-29435 | TMC4 | 9697-9711 | 19878-19892 | 30012-30026 |
| MGC4859 | 9112-9126 | 19299-19313 | 29436-29450 | PYGB | 9712-9726 | 19893-19907 | 30027-30041 |
| AUTS2 | 9127-9141 | 19314-19328 | 29451-29465 | CDH4 | 9727-9741 | 19908-19922 | 30042-30056 |
| SEMA3D | 9142-9156 | 19329-19343 | 29466-29480 | ARFRP1 | 9742-9756 | 19923-19937 | 30057-30071 |
| ARPC1B | 9157-9171 | 19344-19358 | 29481-29495 | MAP3K7CL | 9757-9771 | 19938-19952 | 30072-30086 |
| LINC02237 | 9172-9186 | 19359-19374 | 29496-29510 | PNPLA3 | 9772-9786 | 19953-19967 | 30087-30101 |
| TRIBI | 9187-9201 | 19374-19388 | 29511-29525 | ADIPOQ | 9787-9801 | 19968-19982 | 30102-30116 |
| PTPRD | 9202-9216 | 19389-19403 | 29526-29540 | LIPE | 9802-9816 | 19983-19997 | 30117-30131 |
| TTC39B | 9217-9231 | 19404-19418 | 29541-29555 | UCP1 | 9817-9831 | 19998-20012 | 30132-30146 |
| BNC2 | 9232-9246 | 19419-91433 | 29556-29570 | HSD17B13 | 9832-9846 | 20013-20027 | 30147-30161 |
| MIR12117 | 9247-9261 | 19434-19448 | 29571-29576 | MTARC2 | 9847-9861 | 20028-20042 | 30162-30176 |
| GABBR2 | 9262-9276 | 19449-19463 | 29577-29591 | MLXIP | 9862-9876 | 20043-20057 | 30177-30191 |
| TOR1B | 9277-9291 | 19464-19478 | 29592-29606 | LYPLAL1 | 9877-9891 | 20058-20072 | 30192-30206 |
| ABO | 9292-9306 | 19479-19493 | 29607-29621 | TOR1AIP1 | 9892-9906 | 20073-20087 | 30207-30221 |
| PCAT5 | 9307-9321 | 19494-19508 | 29622-29636 | MLXIPL | 9907-9921 | 20088-20102 | 30222-30236 |
| ZNF487 | 9322-9336 | 19509-19523 | 29637-29651 | CPT2 | 9922-9936 | 20103-20117 | 30237-30251 |
| KCNMA1-AS1 | 9337-9351 | 19524-19538 | 29652-29666 | PPARG | 9937-6651 | 20118-20132 | 30252-30266 |
| CWF19L1 | 9352-9366 | 19539-19553 | 29667-29681 | TOR1AIP2 | 9952-9966 | 20133-20147 | 30267-30281 |
| GPAM | 9367-9381 | 19554-19568 | 29682-29696 | CPT1A | 9967-9981 | 20148-20162 | 30282-30296 |
| SYCE1 | 9382-9396 | 19569-19583 | 29697-29711 | LMNA | 9982-9996 | 20163-20177 | 30297-30311 |
| MIR100HG | 9397-9411 | 19584-19598 | 29712-29726 | ACAA2 | 9997-10011 | 20178-20192 | 30312-30326 |
| PLEKHA5 | 9412-9426 | 19599-19613 | 29727-29741 | SUN1 | 10012-10026 | 20193-20207 | 30327-30341 |
| SLCO1A2 | 9427-9441 | 19614-19622 | 29742-29756 | GRB14 | 10027-10041 | 20208-20222 | 30342-30356 |
| LINC02426 | 9442-9456 | 19623-19637 | 29757-29771 | TMPO | 10042-10056 | 20223-20237 | 30357-30371 |
| SLC6A15 | 9457-9471 | 19638-19652 | 29772-29786 | HSD17B11 | 10057-10071 | 20238-20252 | 30372-30386 |
| LINC02392 | 9472-9486 | 19653-19667 | 29787-29801 | ERLIN1 | 10072-10086 | 20253-20267 | 30387-30401 |
| NEDD1 | 9487-9501 | 19668-19682 | 29802-29816 | PRKAA1 | 10087-10101 | 20268-20282 | 30402-30416 |
| ZNF664- | 9502-9516 | 19683-19698 | 29817-29831 | FASN | 10102-10116 | 20283-20297 | 30417-30431 |
| RFLNA | | | | SERPINA1 | 10117-10131 | 20298-20312 | 30432-30446 |
| DLEU1 | 9517-9531 | 19698-19712 | 29832-29846 | APOB | 10132-10146 | 20313-20327 | 30447-30461 |
| ARGLU1 | 9532-9546 | 19713-19727 | 29847-29861 | MIR4792 | — | — | 30462-30465 |
| PRKD1 | 9547-9561 | 19728-19742 | 29862-29876 | MIR4264 | — | — | 30466-30469 |
| HCN4 | 9562-9576 | 19743-19757 | 29877-29891 | LOC100289187 | — | — | 30470-30472 |
| FTO | 9577-9591 | 19758-19772 | 29892-29906 | MIR5093 | — | — | 30473-30476 |
| MYO15A | 9592-9606 | 19773-19787 | 29907-29921 | MIR3180-1 | — | — | 30477-30480 |
| MIR34c | — | — | 30489-30492 | MIR5000 | — | — | 30481-30484 |
| LOC100287534 | — | — | 30493-30495 | MIR193b | — | — | 30485-30488 |
| MIR3678 | — | — | 30496-30499 | MIR4461 | — | — | 30514-30517 |
| MIR3159 | — | — | 30500-30503 | MIR5701-1 | — | — | 30518-30521 |
| MIR4673 | — | — | 30504-30507 | MIR4787 | — | — | 30522-30525 |
| LOC283403 | — | — | 30508-30510 | MIR1224 | — | — | 30526-30529 |
| LOC100287399 | — | — | 30511-30513 | MIR4728 | — | — | 30532-30535 |
| | | | | MIR101-1 | — | — | 30536-30539 |

### REFERENCES

1. Lazo M, Hernaez R, Eberhardt M S, et al. Prevalence of nonalcoholic fatty liver disease in the United States: the Third National Health and Nutrition Examination Survey, 1988-1994. Am J Epidemiol 2013; 178:38-45.
2. Portillo Sanchez P, Bril F, Maximos M, et al. High Prevalence of Nonalcoholic Fatty Liver Disease in Patients with Type 2 Diabetes Mellitus and Normal Plasma Aminotransferase Levels. J Clin Endocrinol Metab 2014; 100.
3. Crespo J, Fernandez-Gil P, Hernandez-Guerra M, et al. Are there predictive factors of severe liver fibrosis in morbidly obese patients with non-alcoholic steatohepatitis? Obes Surg 2001; 11:254-7.
4. Younossi Z M, Blissett D, Blissett R, et al. The economic and clinical burden of nonalcoholic fatty liver disease in the United States and Europe. Hepatology 2016; 64:1577-1586.
5. Romeo S, Kozlitina J, Xing C, et al. Genetic variation in PNPLA3 confers susceptibility to nonalcoholic fatty liver disease. Nat Genet 2008; 40:1461-5.
6. Speliotes E K, Yerges-Armstrong L M, Wu J, et al. Genome-wide association analysis identifies variants associated with nonalcoholic fatty liver disease that have distinct effects on metabolic traits. PLOS Genet 2011; 7: e1001324.
7. Luukkonen P K, Juuti A, Sammalkorpi H, et al. MARC1 variant rs2642438 increases hepatic phosphatidylcholines and decreases severity of non-alcoholic fatty liver disease in humans. J Hepatol 2020; 73:725-726.
8. Parisinos C A, Wilman H R, Thomas E L, et al. Genome-wide and Mendelian randomisation studies of liver MRI yield insights into the pathogenesis of steatohepatitis. J Hepatol 2020; 73:241-251.
9. Middleton M S, Heba E R, Hooker C A, et al. Agreement Between Magnetic Resonance Imaging Proton Density Fat Fraction Measurements and Pathologist-Assigned Steatosis Grades of Liver Biopsies From Adults With Nonalcoholic Steatohepatitis. Gastroenterology 2017; 153:753-761.
10. Saadeh S, Younossi Z M, Remer E M, et al. The utility of radiological imaging in nonalcoholic fatty liver disease. Gastroenterology 2002; 123:745-50.
11. Harris T B, Launer L J, Eiriksdottir G, et al. Age, Gene/Environment Susceptibility-Reykjavik Study: multidisciplinary applied phenomics. Am J Epidemiol 2007; 165:1076-87.
12. Regan E A, Hokanson J E, Murphy J R, et al. Genetic epidemiology of COPD (COPDGene) study design. COPD 2010; 7:32-43.
13. Carr J J, Nelson J C, Wong N D, et al. Calcified coronary artery plaque measurement with cardiac C T in population-based studies: standardized protocol of Multi-Ethnic Study of

Atherosclerosis (MESA) and Coronary Artery Risk Development in Young Adults (CARDIA) study. Radiology 2005; 234:35-43.
14. Speliotes E K, Massaro J M, Hoffmann U, et al. Liver fat is reproducibly measured using computed tomography in the Framingham Heart Study. J Gastroenterol Hepatol 2008; 23:894-9.
15. Daniels P R, Kardia S L, Hanis C L, et al. Familial aggregation of hypertension treatment and control in the Genetic Epidemiology Network of Arteriopathy (GENOA) study. Am J Med 2004; 116:676-81.
16. Palmer N D, Goodarzi M O, Langefeld C D, et al. Genetic Variants Associated With Quantitative Glucose Homeostasis Traits Translate to Type 2 Diabetes in Mexican Americans; The GUARDIAN (Genetics Underlying Diabetes in Hispanics) Consortium. Diabetes 2015; 64:1853-66.
17. Liu J, Musani S K, Bidulescu A, et al. Fatty liver, abdominal adipose tissue and atherosclerotic calcification in African Americans: the Jackson Heart Study. Atherosclerosis 2012; 224:521-5.
18. Kramer H, Han C, Post W, et al. Racial/ethnic differences in hypertension and hypertension treatment and control in the multi-ethnic study of atherosclerosis (MESA). Am J Hypertens 2004; 17:963-70.
19. Rampersaud E, Bielak L F, Parsa A, et al. The association of coronary artery calcification and carotid artery intima-media thickness with distinct, traditional coronary artery disease risk factors in asymptomatic adults. Am J Epidemiol 2008; 168:1016-23.
20. Canela-Xandri O, Rawlik K, Tenesa A. An atlas of genetic associations in UK Biobank. Nature Genetics 2018; 50:1593-1599.
21. K. He X Z, S. Ren and J. Sun. Deep Residual Learning for Image Recognition. 2016 IEEE Conference on Computer Vision and Pattern Recognition (CVPR), Las Vegas, NV 2016:770-778.
22. Namjou B, Lingren T, Huang Y, et al. GWAS and enrichment analyses of non-alcoholic fatty liver disease identify new trait-associated genes and pathways across eMERGE Network. BMC Med 2019; 17:135.
23. Chen V L, Chen Y, Du X, et al. Genetic variants that associate with cirrhosis have pleiotropic effects on human traits. Liver Int 2020; 40:405-415.
24. Willer C J, Li Y, Abecasis G R. METAL: fast and efficient meta-analysis of genomewide association scans. Bioinformatics 2010; 26:2190-2191.
25. Zhou W, Nielsen J B, Fritsche L G, et al. Efficiently controlling for case-control imbalance and sample relatedness in large-scale genetic association studies. Nat Genet 2018; 50:1335-1341.
26. Dongiovanni P, Valenti L, Rametta R, et al. Genetic variants regulating insulin receptor signalling are associated with the severity of liver damage in patients with non-alcoholic fatty liver disease. Gut 2010; 59:267-73.
27. Feitosa M F, Wojczynski M K, North K E, et al. The ERLIN1-CHUK-CWF19L1 gene cluster influences liver fat deposition and hepatic inflammation in the NHLBI Family Heart Study. Atherosclerosis 2013; 228:175-80.
28. Chalasani N, Guo X, Loomba R, et al. Genome-wide association study identifies variants associated with histologic features of nonalcoholic Fatty liver disease. Gastroenterology 2010; 139:1567-76, 1576 e1-6.
29. Eslam M, Hashem A M, Leung R, et al. Interferon-lambda rs12979860 genotype and liver fibrosis in viral and non-viral chronic liver disease. Nat Commun 2015; 6:6422.
30. Wiedmann S, Fischer M, Kochler M, et al. Genetic variants within the LPIN1 gene, encoding lipin, are influencing phenotypes of the metabolic syndrome in humans. Diabetes 2008; 57:209-17.
31. Shang X R, Song J Y, Liu P H, et al. GWAS-Identified Common Variants With Nonalcoholic Fatty Liver Disease in Chinese Children. J Pediatr Gastroenterol Nutr 2015; 60:669-74.
32. Petta S, Grimaudo S, Camma C, et al. IL28B and PNPLA3 polymorphisms affect histological liver damage in patients with non-alcoholic fatty liver disease. J Hepatol 2012:56:1356-62.
33. Kitamoto T, Kitamoto A, Yoneda M, et al. Genome-wide scan revealed that polymorphisms in the PNPLA3, SAMM50, and PARVB genes are associated with development and progression of nonalcoholic fatty liver disease in Japan. Hum Genet 2013; 132:783-92.
34. Anstee Q M, Darlay R, Cockell S, et al. Genome-wide association study of non-alcoholic fatty liver and steatohepatitis in a histologically characterised cohort ( ) J Hepatol 2020; 73:505-515.
35. Mancina R M, Dongiovanni P, Petta S, et al. The MBOAT7-TMC4 Variant rs641738 Increases Risk of Nonalcoholic Fatty Liver Disease in Individuals of European Descent. Gastroenterology 2016; 150:1219-1230 e6.
36. Ma Y, Belyaeva O V, Brown P M, et al. 17-Beta Hydroxysteroid Dehydrogenase 13 Is a Hepatic Retinol Dehydrogenase Associated With Histological Features of Nonalcoholic Fatty Liver Disease. Hepatology 2019; 69:1504-1519.
37. Park S L, Li Y, Sheng X, et al. Genome-Wide Association Study of Liver Fat: The Multiethnic Cohort Adiposity Phenotype Study. Hepatol Commun 2020; 4:1112-1123.
38. Chen V L, Du X, Chen Y, et al. Genome-wide association study of serum liver enzymes implicates diverse metabolic and liver pathology. Nat Commun 2021; 12:816.
39. Neale B. http://www.nealelab.is/uk-biobank/.
40. Charrad M, Ghazzali N, Boiteau V, et al. NbClust: An R Package for Determining the Relevant Number of Clusters in a Data Set. Journal of Statistical Software 2014; 61:1-36.
41. Galili T. dendextend: an R package for visualizing, adjusting and comparing trees of hierarchical clustering. Bioinformatics 2015; 31:3718-20.
42. Hemani G, Zheng J, Elsworth B, et al. The MR-Base platform supports systematic causal inference across the human phenome. Elife 2018; 7.
43. Lawlor D A, Harbord R M, Sterne J A, et al. Mendelian randomization: using genes as instruments for making causal inferences in epidemiology. Stat Med 2008; 27:1133-63.
44. Pers T H, Karjalainen J M, Chan Y, et al. Biological interpretation of genome-wide association studies using predicted gene functions. Nat Commun 2015; 6:5890.
45. Andri S. DescTools: Tools for Descriptive Statistics. R package version 0.99.40, 2021.
46. Lumley T, Brody J, Dupuis J, et al. Meta-analysis of a rare-variant association test. Stat Tech, University of Auckland 2012.
47. Lee S, Emond M J, Bamshad M J, et al. Optimal unified approach for rare-variant association testing with application to small-sample case-control whole-exome sequencing studies. Am J Hum Genet 2012; 91:224-37.
48. Yates A D, Achuthan P, Akanni W, et al. Ensembl 2020. Nucleic Acids Res 2020; 48: D682-D688.
49. Zhou W, Zhao Z, Nielsen J B, et al. Scalable generalized linear mixed model for region-based association tests in large biobanks and cohorts. Nat Genet 2020; 52:634-639.
50. Kahali B, Chen Y, Feitosa M F, et al. A Noncoding Variant Near PPP1R3B Promotes Liver Glycogen Storage and MetS, but Protects Against Myocardial Infarction. J Clin Endocrinol Metab 2021; 106:372-387.
51. Landgraf K, Scholz M, Kovacs P, et al. FTO Obesity Risk Variants Are Linked to Adipocyte IRX3 Expression and BMI of Children-Relevance of FTO Variants to Defend Body Weight in Lean Children? PLOS One 2016; 11: e0161739.
52. Liberzon A, Subramanian A, Pinchback R, et al. Molecular signatures database (MSigDB) 3.0. Bioinformatics 2011; 27:1739-40.
53. Consortium G T. The GTEx Consortium atlas of genetic regulatory effects across human tissues. Science 2020; 369:1318-1330.
54. Polimanti R, Gelernter J. ADH1B: From alcoholism, natural selection, and cancer to the human phenome. Am J Med Genet B Neuropsychiatr Genet 2018; 177:113-125.
55. Muenter M D, Perry H O, Ludwig J. Chronic vitamin A intoxication in adults. Hepatic, neurologic and dermatologic complications. Am J Med 1971; 50:129-36.
56. Shin J Y, Hernandez-Ono A, Fedotova T, et al. Nuclear envelope-localized torsinA-LAP1 complex regulates hepatic VLDL secretion and steatosis. J Clin Invest 2019; 129:4885-4900.
57. Innes H, Buch S, Hutchinson S, et al. Genome-Wide Association Study for Alcohol-Related Cirrhosis Identifies Risk Loci in MARC1 and HNRNPUL1. Gastroenterology 2020; 159:1276-1289 e7.
58. Xia M, Chandrasekaran P, Rong S, et al. Hepatic Deletion of Mboat7 (Lpiat1) Causes Activation of SREBP-Ic and Fatty Liver. J Lipid Res 2020.
59. Chen Y, Chen C, Ke X, et al. Analysis of circulating cholesterol levels as a mediator of an association between ABO blood group and coronary heart disease. Circ Cardiovasc Genet 2014; 7:43-8.
60. Wolpin B M, Kraft P, Xu M, et al. Variant ABO blood group alleles, secretor status, and risk of pancreatic cancer: results from the pancreatic cancer cohort consortium. Cancer Epidemiol Biomarkers Prev 2010; 19:3140-9.
61. Zhong G C, Liu S, Wu Y L, et al. ABO blood group and risk of newly diagnosed nonalcoholic fatty liver disease: A case-control study in Han Chinese population. PLOS One 2019; 14: e0225792.
62. Chambers J C, Zhang W, Sehmi J, et al. Genome-wide association study identifies loci influencing concentrations of liver enzymes in plasma. Nat Genet 2011; 43:1131-8.
63. Kathiresan S, Melander O, Guiducci C, et al. Six new loci associated with blood low-density lipoprotein cholesterol, high-density lipoprotein cholesterol or triglycerides in humans. Nat Genet 2008; 40:189-97.
64. Beer N L, Tribble N D, McCulloch L J, et al. The P446L variant in GCKR associated with fasting plasma glucose and triglyceride levels exerts its effect through increased glucokinase activity in liver. Hum Mol Genet 2009; 18:4081-8.
65. Ishizuka Y, Nakayama K, Ogawa A, et al. TRIB1 downregulates hepatic lipogenesis and glycogenesis via multiple molecular interactions. J Mol Endocrinol 2014; 52:145-58.
66. Bauer R C, Sasaki M, Cohen D M, et al. Tribbles-1 regulates hepatic lipogenesis through posttranscriptional regulation of C/EBPalpha. J Clin Invest 2015; 125:3809-18.
67. Agius L. Hormonal and Metabolite Regulation of Hepatic Glucokinase. Annu Rev Nutr 2016; 36:389-415.
68. Nakajima S, Tanaka H, Sawada K, et al. Polymorphism of receptor-type tyrosine-protein phosphatase delta gene in the development of non-alcoholic fatty liver disease. J Gastroenterol Hepatol 2018; 33:283-290.
69. Kozlitina J, Smagris E, Stender S, et al. Exome-wide association study identifies a TM6SF2 variant that confers susceptibility to nonalcoholic fatty liver disease. Nat Genet 2014; 46:352-6.
70. Wang Y, Kory N, BasuRay S, et al. PNPLA3, CGI-58, and Inhibition of Hepatic Triglyceride Hydrolysis in Mice. Hepatology 2019; 69:2427-2441.
71. Palmer N, Kahali B, Kuppa A, et al. Allele Specific Variation at APOE Increases Non-alcoholic Fatty Liver Disease and Obesity but Decreases Risk of Alzheimer's Disease and Myocardial Infarction. 2021.
72. Hannah V C, Ou J, Luong A, et al. Unsaturated fatty acids down-regulate srebp isoforms 1a and 1 by two mechanisms in HEK-293 cells. J Biol Chem 2001; 276:4365-72.
73. Abul-Husn N S, Cheng X, Li A H, et al. A Protein-Truncating HSD17B13 Variant and Protection from Chronic Liver Disease. N Engl J Med 2018; 378:1096-1106.
74. Fox C S, Liu Y, White C C, et al. Genome-wide association for abdominal subcutaneous and visceral adipose reveals a novel locus for visceral fat in women. PLoS Genet 2012; 8: e1002695.
75. Sirwi A, Hussain M M. Lipid transfer proteins in the assembly of apoB-containing lipoproteins. J Lipid Res 2018; 59:1094-1102.
76. Burnett J R, Hooper A J, Hegele R A. Abetalipoproteinemia. In: Adam M P, Ardinger H H, Pagon R A, Wallace S E, Bean LJH, Mirzaa G, Amemiya A, eds. GeneReviews ((R)). Seattle (WA), 1993.

1. A method comprising: analyzing a biological sample from a subject for ten to one hundred variants, wherein at least ten of the variants are from the list of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, and mutations in MTTP.

2. The method of claim 1, wherein said at least ten of the variants comprises at least fifteen of the variants from the list of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, and mutations in MTTP.

3. The method of claim 1, wherein said at least ten of the variants comprises each of the variants from the list rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, and mutations in MTTP.

4. The method of claim 1, wherein said at least ten of the variants consists of only the variants from the list of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, and mutations in MTTP.

5. The method of claim 1, wherein said at least ten of the variants consists of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, and mutations in MTTP.

6. The method of any of claims 1-5, wherein said biological sample is obtained from a subject suspected of having nonalcoholic fatty liver disease.

7. The method of any of claims 1-6, wherein said biological sample is selected from the group consisting of blood, serum, plasma, saliva, tissue, hair, semen, and urine.

8. The method of any of claims 1-7, wherein said analyzing comprises directly detecting said variants using a molecule assay.

9. The method of claim 8, wherein the molecule assay is a hybridization assay or a sequencing assay.

10. The method of any of claims 1-9, wherein said analyzing comprises indirectly detecting said variants.

11. The method of claim 10, wherein said indirectly detecting comprises assessing gene expression or detecting a mutation in linkage disequilibrium with a variant.

12. A method of managing nonalcoholic fatty liver disease, comprising:
a) analyzing a biological sample from a subject for at least ten of the variants from the list of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, and mutations in MTTP;
b) generating a fatty liver disease risk score based on the presence or absence of said variants; and
c) treating the subject with a nonalcoholic fatty liver disease intervention if said risk score indicates a predisposition to nonalcoholic fatty liver disease.

13. The method of claim 12, wherein said risk score is calculated using an algorithm that accounts for each of the analyzed variants.

14. The method of claim 12 or 13, wherein said risk score further is based on one or more of blood count, liver enzyme test data, liver function test data, hepatitis A test data, hepatitis C test data, celiac disease screening test data, fasting blood sugar, hemoglobin A1C data, and lipid profile data.

15. The method of any of claims 12-14, wherein said risk score further is based on one or more of abdominal ultrasound data, computerized tomography (CT) scanning data, magnetic resonance imaging (MRI) data, transient elastography data, and magnetic resonance elastography data.

16. The method of any of claims 12-15, wherein said treating comprises applying a weight loss regime.

17. The method of any of claims 12-16, wherein said treating comprises liver transplantation.

18. The method of any of claims 12-17, wherein said treating comprises administration of one or more active agents selected from the group consisting of an essential phospholipid; anti-diabetic agent; a dietary supplement; an antifibrotic agent; an anti-obesity agent; and any combination thereof.

19. A system comprising: a set or reagents that specifically detect ten to one hundred variants, wherein at least ten of the variants are from the list of rs738408, rs58542926, rs429358, rs 1260326, rs28601761, rs4918722, rs2807834, rs7661964, rs1229984, rs7029757, rs17817449, rs79953491, rs112630404, rs626283, rs4561528, rs10756038, rs140201358, or a variant of marker in linkage disequilibrium therewith, and mutations in MTTP.

20. The system of claim 19, wherein said reagents comprises one or more primers or probe specific for said variants.

21. The system of claim 19 or 20, wherein said reagents comprising sequence reagents.

22. The system of any of claims 19-21, wherein said reagents comprises a microarray.

23. A non-transitory computer-readable storage medium comprising an instruction, wherein when the instruction is run by at least one computer processor, wherein the at least one processor performs operations comprising: a) receiving data identifying the presence or absence of a variant in a biological sample from at least ten of from the list of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, or a variant or marker in linkage disequilibrium therewith, and mutations in MTTP; b) generating a nonalcoholic fatty acid liver disease risk score from said data; and c) displaying or reporting said risk score.

25. A method of diagnosing fatty liver disease or predisposition to fatty liver disease comprising: analyzing a biological sample from a subject for at least ten variants from the list of rs738408, rs58542926, rs429358, rs1260326, rs28601761, rs4918722, rs2807834, rs7661964, rs1229984, rs7029757, rs17817449, rs79953491, rs112630404, rs626283, rs4561528, rs10756038, rs140201358, or a variant or marker in linkage disequilibrium therewith, and mutations in MTTP.
